(19) United States
(12) Patent Application Publication
(10) Pub. No.: US 2026/0234553 A1
(43) Pub. Date: Aug. 13, 2026

(54) COMPOSITIONS COMPRISING SUPRAMOLECULAR ASSEMBLIES OF PEPTIDE AMPHIPHILES AND ORGANIC CATIONS AND METHODS OF USE THEREOF

(71) Applicant: Northwestern University, Evanston, IL (US)

(72) Inventors: Zois SYRGIANNIS, Evanston, IL (US); Nicholas Allen SATHER, Evanston, IL (US); Ivan SASSELLI RAMOS, Evanston, IL (US); Zaida Alvarez Pinto, Evanston, IL (US); Saumel I. Stupp, Evanston, IL (US)

(21) Appl. No.: 19/147,007

(22) PCT Filed: Jan. 16, 2024
(86) PCT No.: PCT/US2024/011665

§ 371 Date: Jul. 10, 2025

Related U.S. Application Data
(60) Provisional application No. 63/480,084, filed on Jan. 16, 2023.

(51) Int. Cl. C12N 5/0793; C07K 7/06; C07K 7/08; C12N 5/00

(52) U.S. Cl. CPC C12N 5/0619; C07K 7/06; C07K 7/08; C12N 5/0018; C12N 2501/115; C12N 2501/13; C12N 2501/15; C12N 2501/155; C12N 2501/165

(57) ABSTRACT

Provided herein are compositions comprising supercharged supramolecular polymers capable of generating dense ion clouds and methods of use thereof, for example, to enhance neuronal development and membrane excitability. In particular, compositions herein comprise co-assemblies of peptide amphiphiles and organic cations.

### CROSS-REFERENCE TO RELATED APPLICATIONS

This application claims the benefit of U.S. Provisional Patent Application No. 63/480,084, filed on Jan. 16, 2023, which is incorporated by reference herein.

### STATEMENT REGARDING FEDERAL FUNDING

This invention was made with government support under grant number CHE-2102662 awarded by the National Science Foundation. The government has certain rights in the invention.

### FIELD

Provided herein are compositions comprising supercharged supramolecular polymers capable of generating dense ion clouds and methods of use thereof, for example, to enhance neuronal development and membrane excitability. In particular, compositions herein comprise co-assemblies of peptide amphiphiles and organic cations.

### BACKGROUND

Ion transport and storage have been a central theme in science, given its implications in energy systems and critical role in biological function. These phenomena are, of course, essential in the ongoing development of batteries, and biologically dominate the transmission of electrical signals in the central nervous system (CNS). Whereas in batteries and energy storage devices, the key objective is always to improve ion transport efficiency within materials, in biological systems, complex autonomous processes regulate functional ion flow and availability. Focusing on biological systems, the widespread distribution of more than 100 ion-specific channels and their temporal activation plays a vital role in physiological function. When ion channel function is compromised as a result of genetic mutations or external factors, many serious conditions referred to as channelopathies can emerge, which cause diseases or loss of function ranging from epilepsies to cystic fibrosis and blindness, among many others.

To control ion flow in the CNS there has been great interest in developing therapeutic strategies that utilize synthetic molecules. The primary approach involves the use of artificial transporters which bypass endogenous ion channels by binding specific ions and transferring them through the hydrophobic bilayer of cell membranes. These ion-selective molecules have demonstrated moderate bioactivity but are limited by low amounts of total ions transferred. In biological systems, neurons utilize glial cells to maintain buffered reservoirs of key ions essential for proper neurotransmission.

### SUMMARY

Provided herein are compositions comprising supercharged supramolecular polymers capable of generating dense ion clouds and methods of use thereof, for example, to enhance neuronal development and membrane excitability. In particular, compositions herein comprise co-assemblies of peptide amphiphiles and organic cations.

To manipulate ion transport dynamics, experiments were conducted during development of embodiments herein to develop a functional material by co-assembling anionic peptide amphiphiles (PA) with small organic cations (OCs). The combined theoretical computational and experimental approach revealed that PA molecules organized within PA/OC nanofiber assemblies have a higher degree of ionization than those found within PA fibrils. Therefore, PA/OC scaffolds recruit highly dense ion clouds around their surfaces, which stimulate evolutionary conserved neuronal gene expression programs. Besides the fundamental importance of such scaffolds to elucidate ion transfer mechanisms, these co-assembled nanostructures enhance neural development and membrane excitability through unique molecular mechanisms, thus revealing utility as regenerative therapies.

In some embodiments, provided herein are compositions comprising peptide amphiphiles (PAS) and organic cations (OCs). In some embodiments, the PAs are self-assembled into a supramolecular nanofiber. In some embodiments, the PAs and OCs co-assemble into nanofibers having distinct geometries from nanofibers of the PAs alone. In some embodiments, PAs within PA/OC co-assemblies exhibit a greater degree (e.g., 5% increase, 10% increase, 20% increase, 30% increase, 40% increase, 50% increase, or more) of deprotonation of amino acids in the charged peptide segment than PAs in the absence of OC co-assembly under the same extrinsic conditions. In some embodiments, for PAs comprising charged peptide segments of 2 amino acids in length (e.g., EE) at least 70% (e.g., 75%, 80%, 85%, 90%, 95%, 98%, 99%, 99.9%, 100%, or ranges therebetween) of the PAs in the co-assembled nanofibers are fully ionized (e.g., deprotonated). In some embodiments, for PAs comprising charged peptide segments of 3 amino acids or more in length (e.g., EEE, EEEE) at least 70% (e.g., 75%, 80%, 85%, 90%, 95%, 98%, 99%, 99.9%, 100%, or ranges therebetween) of the PAs in the co-assembled nanofibers have the terminal two amino acids (e.g., deprotonated). In some embodiments, the co-assembly does not result in gelation or aggregation of nanofibers of the PAs. In some embodiments, the PAs comprise: (a) a hydrophobic non-peptide segment; (b) a structural peptide segment; and (c) a charged peptide segment. In some embodiments, the hydrophobic segment is linked to the structural peptide segment. In some embodiments, the structural peptide segment is linked to the charged peptide segment. In some embodiments, the hydrophobic non-peptide segment comprises an acyl chain. In some embodiments, the acyl chain comprises C₆-C₂₀ (e.g., C₆, C₈, C₁₀, C₁₂, C₁₄, C1₆, C₁₈, C₂₀, or ranges therebetween). In some embodiments, the structural peptide segment is a beta-sheet forming peptide segment. In some embodiments, the structural peptide segment is an alanine-, glycine-, and/or valine-rich peptide segment. In some embodiments, the alanine-, glycine-, and/or valine-rich peptide segment comprises AAVV (SEQ ID NO: 1), AAAVVV (SEQ ID NO: 2) VVAA (SEQ ID NO: 3), VVVAAA (SEQ ID NO: 4), AAGG (SEQ ID NO: 5), and/or GGAA (SEQ ID NO: 6). In some embodiments, the charged peptide segment is a glutamate- and/or aspartate-rich segment. In some embodiments, the glutamate- and/or aspartate-rich segment comprises 2-7 amino acids in length with 50% or more (e.g., 60%, 70%, 80%, 90%) amino acids selected from Glu (E) and/or Asp (D) residues. In some embodiments, the glutamate- and/or aspartate-rich segment comprises EE, EEE, or EEEE. In some embodiments, the PA comprises the sequence VVAAEE (SEQ ID NO: 7) or AAGGEE (SEQ ID NO: 8). In some embodiments, all or a portion of the PAs further comprise (d) a bioactive peptide. In some embodiments, the bioactive peptide is selected from a VEGF mimetic peptide (e.g., IKVAV (SEQ ID NO: 9)), a FGF2 mimetic peptide, a TGF-β1 mimetic peptide, BDNF mimetic peptide, Netrin-1 peptide, and BMP-2 binding peptide (e.g., TSPHVPYGGGS (SEQ ID NO: 10)). In some embodiments, PAs with a bioactive peptide and without a bioactive peptide are present at a ratio between 1:10 and 10:1 (e.g., 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1). In some embodiments, the OCs are selected from ethyl pyridinium, N-ethyl-N-methyl pyrrolidinium, trimethylsulfonium, tetramethylammonium, methylammonium, formamidinium, aziridiium, cyclopropenium, ethylenediamine, allyl methyl imidazolium, butyl methyl imidazolium, and ethyl methyl imidazolium. In some embodiments, the OCs are 1-ethyl-3-methylimidazolium. In some embodiments, the OCs are small molecules, having molecular weights of 500 g/mol or less. In some embodiments, are capable of forming non-covalent interactions with non-polar amino acids (e.g., G, A, V, etc.). In some embodiments, the OCs are monocationic. In some embodiments, the OCs exhibit a distributed charge profile. In some embodiments, the composition comprises 1-50 molar equivalents of OC per PA (e.g., 1:1 to 1:50 molar ratio of OC to PA (e.g., 1:1, 1:2, 1:5: 1:10, 1:15, 1:20, 1:25, 1:30, 1:35, 1:40, 1:45, 1:50, or ranges or ratios therebetween)).

In some embodiments, compositions herein further comprise one or more types of inorganic cations. In some embodiments, the inorganic cations form a cloud around a nanofiber co-assembly of peptide amphiphiles and organic cations. In some embodiments, the inorganic cations are selected from K⁺, Na⁺, Rb⁺, Cs⁺, Ca²⁺, Mg²⁺, Zn²⁺, Sc³⁺, and Mn²⁺. In some embodiments, the inorganic cations are monoanionic. In some embodiments, the inorganic cations form an ion cloud around the supramolecular nanofibers.

In some embodiments, provided herein are compositions comprising: (a) a peptide amphiphile comprising: (i) a hydrophobic non-peptide segment, (ii) a structural peptide segment, and (iii) a charged peptide segment; (b) an organic cation; and (c) inorganic cations.

In some embodiments, provided herein are compositions comprising: (a) peptide amphiphile comprising: (i) a hydrophobic non-peptide segment, (ii) a structural peptide segment, (iii) a charged peptide segment, and (iv) a bioactive peptide segment; (b) an organic cation; and (c) inorganic cations.

In some embodiments, provided herein are compositions comprising: (a) a first peptide amphiphile comprising: (i) a hydrophobic non-peptide segment, (ii) a structural peptide segment, and (iii) a charged peptide segment; (b) a second peptide amphiphile comprising: (i) a hydrophobic non-peptide segment, (ii) a structural peptide segment, (iii) a charged peptide segment, and (iv) a bioactive peptide segment; (c) an organic cation; and (d) inorganic cations.

In some embodiments, provided herein are methods of enhancing the development of neurons in vitro comprising contacting the neurons with the PA/OC co-assembly compositions herein. In some embodiments, provided herein are methods of enhancing the membrane excitability of neurons in vitro comprising contacting the PA/OC co-assembly compositions herein. In some embodiments, the PA/OC co-assembly compositions herein are included in neuron cell culture media. In some embodiments, a substate upon which the neurons are cultured comprises a PA/OC co-assembly compositions herein.

### BRIEF DESCRIPTION OF THE DRAWINGS

FIGS. 1A-J. Organic cation-peptide amphiphile (OC-PA) nanostructures and their impact on neuronal maturation. (A) Chemical structures of PA (left) and OC (1-ethyl-3-methyl imidazolium chloride; right). (B) Cryo-TEM micrographs of OC-PA nanoribbon (scale bar, 100 nm). Inset highlights twisted AFM topographic images representing the OC-PA (scale bar, 50 nm). (C) Electrophoretic mobility from z-potential measurements for PA and OC-PA. (D) Schematic representation of the PA nanostructure with organic cations bound within the nanoribbons (OC_{c}). Region of ¹H NMR spectra featuring α- and β-hydrogens of OC i) free in solution (OC_{f}), and ii) OC-PA gel pellet (slow exchange of OC at the surface of the nanostructure and inside the nanostructure, OCₛ⇄OC_{c}) obtained using HR-MAS probe (800 MHz, 310 K, 10 KHz MAS, PBS, 10% D₂O) (H_{f}: α protons from free OC; Hₛ: α protons from OC that interacts electrostatically with PA nanostructures; H_{c}: α protons from OC that are in PA nanostructures) spectra shown in (i) is recorded using solution NMR probe (600 MHz, 298 K, PBS, 10% D₂O). (E) Representative confocal micrographs of mouse E16.5 (E, embryonic day) cortical neurons cultured for 24 hours on PA (left) or OC-PA (right) immunolabeled for b-tubulin (neuronal marker) and F-Actin (growth cone marker), and counterstained with the nuclear marker DAPI. Scale bar, 20 mm. (F) Growth cone area quantitation from panel E. n=50-60 neurons per condition, ****p<0.0001, student's two-tailed t-test. (G) Bright-field images of cortical neurons cultured on PA (left) or OC-PA (right) after 6 DIV. Neurites were colored using IncuCyte® Live-Cell Analysis System. Scale bar, 20 mm. (H) Representative fluorescent micrographs of neuronal networks from cortical neurons grown for 14 DIV and immunostained for MAP-2 (mature neuronal dendritic marker) and NeuN (pan-neuronal nuclear marker), and counterstained with DAPI. Scale bar, 20 mm. (I) Representative traces of elicited action potentials (AP) measured using direct current injection ramps (50-400 pA, 500 ms). Recordings from cortical neurons cultured on PA or OC-PA for 12-14 DIV. (J) Plot representing synchrony index in neurons cultured on PA and OC-PA.

FIGS. 2A-E. OC-PA co-assemblies have higher ionization than pure PA assemblies. (A) Segments of ¹H NMR featuring E side chains measured for (i) peptide AcVVAAEE at 10 mM in solution (600 MHz, Cryoprobe, 37° C., 10% D₂O in PBS 1×, pH 7.4), (ii) PA and (iii) OC-PA nanostructures in gel (semi-solid) state (800 MHz, HR-MAS probe, 10 kHz MAS, 37° C., 10% D₂O) isolated by ultracentrifugation of 10 mM annealed PA and OCPA solutions, respectively, prepared in PBS buffer at pH 7.4 in the presence of 10% D₂O. (B) Selected region of ¹H—¹H TOCSY HR-MAS NMR of PA fibers (800 MHz, HR-MAS probe, 10 kHz MAS, 37° C., 10% D₂O) used to assign the side chain protons to the corresponding α-hydrogens from the two sets of E residues in PA. (C) Snapshots from the all-atom MD simulation of the PA (left) and OC-PA (right), which were performed using the GROMACS 2016.3 package. (D) Water molecules concentration vs. PA molecules from all-atom MD simulation. (E) Crossed-section snapshots from coarse-grained MD simulations for PA (top) and OC-PA (bottom).

FIGS. 3A-G. Dense ion clouds form around OC-PA nanostructures (Ion Cloud Nanostructures: ICN). Anomalous small-angle X-ray scattering (ASAXS) measurements of non-resonant and cross-term scattering from Rb⁺ ions around the (A) PA filaments and (B) OC-PA filaments, along with corresponding simulated intensities (with PA: 50% charged and OC-PA: 90% charged). (C) Ion distribution around PA and OC-PA filaments extracted from the geometric fitting of ASAXS profiles. (D) Schematic representation of the ion cloud around PA and ion cloud nanostructures (ICN) filaments. (E) ²³Na NMR spectra for aqueous 145 mM Na⁺ in the presence of PA and ICN materials (158 MHz, 25° C., pH 7.4, 10% D₂O). (F) ²³Na R₁ and R₂ relaxation rates of measured in aqueous solutions of PA and ICN materials; total Na⁺ concentration was kept constant in the two samples. (G) R₁ and R₂ spin relaxation of Na⁺ counterions in ICN aqueous solutions measured upon the incremental addition of K⁺ ions; note that reduction in relaxation rates corroborates Na⁺ exchange with K⁺ ions.

FIGS. 4A-M. ICNs enhance mouse and human neuronal development through CREB-dependent intracellular signaling mechanisms. (A) Mouse E16.5 (E, embryonic day) cortical neurons were plated overnight and treated for 48 hours with PA control, Na⁺-ICN, or K⁺-ICN. Triple-immunolabeling with p37-LRP (laminin receptor protein), pCREB (phosphorylated CAMP response element-binding protein at Serine 133), and b-Tubulin III. Arrows denote p37-LRP⁺ dendritic growth cones, whereas the asterisk denotes a neuron with low pCREB expression. Scale bar, 20 mm. (B) Quantitation of p37-LRP⁺ dendritic growth cones after 48 hours of PA or ICN treatment. ***p<0.001, ns=non-significant, n=50-60 neurons per condition. (C) Quantitation of pCREB⁺ neurons (% of DAPI⁺ nuclei) after 48 hours of PA or ICN treatment. ***p<0.001, ns=non-significant, n=50-60 neurons per condition. (D) Quantitation of average b-Tubulin III⁺ neurite length after 48 hours of PA or ICN treatment. ***p<0.001, ns=non-significant, n=50-60 neurons per condition. (E) Immunoblots and corresponding quantitation with pCREB or total CREB antibodies from E16.5 mouse cortical neuron whole-cell extracts treated with PA or ICN for 14 days. b-actin served as loading control. n=6 per condition. Volcano plots of PA control vs Na⁺-ICN, or K⁺-ICN, highlighting (F) genes involved in mitochondrial oxidative phosphorylation and; (G) genes involved in oxidative phosphorylation and secretory vesicle formation. (H) Counts per million (CPM) normalized read counts for target genes upregulated for both Na⁺-ICN and K⁺-ICN relative to PA control. **p<0.01, n=3 libraries per condition. (I) Counts per million (CPM) normalized read counts for target genes upregulated solely for K⁺-ICN relative to PA control. **p<0.01, ns, non-significant, n=3 libraries per condition. See also Data File S1 for differential expression analyses between all conditions. (J) Naᵥ1.6 immunoreactivity of E16.5 mouse cortical neurons treated with PA or ICN for 6 days. Trim46 labels axon initial segment (AIS). Scale bar, 20 mm. (K) Quantitation of Naᵥ1.6 protein levels from E16.5 mouse cortical neurons treated with PA, Na⁺-ICN, or K⁺-ICN for 6 days. ***p<0.001, ns=non-significant, n=50-60 neurons per condition. (L) pCREB and b-Tubulin III immunolabeling of human glutamatergic neurons derived from iPSCs. Neurons were treated with PA, Na⁺-ICN, or K⁺-ICN for 72 hours. Boxed areas are enlarged on the bottom row showing only the pCREB channel. Scale bar, 20 mm; bottom insets, 5 mm. (M) Quantitation of pCREB protein levels within dendritic branches from bottom panel L. **p<0.01, ****p<0.0001, n=50-60 neurons per condition.

FIGS. 5A-J. ICNs trigger intrinsic and molecular plasticity in hippocampal CA1 pyramidal neurons. (A) Schematics for experimental design of hippocampal organotypic slices on pre-assembled PA or ICN Millicell membranes with recording and experimental timelines. (B) Input-output curves of control CA1 pyramidal neurons cultured on PA or Na⁺-ICN at different time points (DIV 6, 10, 14; Mann-Whitney test, **p<0.005, ***p<0.0005). The right panels show representative traces of control PA neurons vs Na⁺-ICN neurons at different DIV. (C) Rheobase is decreased at all time points in CA1 pyramidal neurons cultured on Na⁺-ICN. Left, pooled data of single neuron recordings (Mann-Whitney test, *p<0.05, **p<0.005). The right panels show representative traces of rheobase of control PA vs Na⁺-ICN (90 to 60 pA at 6 DIV, 140 to 80 pA at 10 DIV, and 130 to 100 pA at 14 DIV). (D) Input-output curves of CA1 pyramidal neurons cultured on PA or K⁺-ICN at different time points (DIV 6, 10, 14; Mann-Whitney test, **p<0.005, ***p<0.0005). The right panels show representative traces of control PA neurons vs K⁺-ICN neurons at different DIV. (E) Rheobase is decreased at all time points in CA1 pyramidal neurons cultured on K⁺-ICN. Left, pooled data of single neuron recordings (Mann-Whitney test *p<0.05, ***p<0.0005). The right panels show representative traces of rheobase of control PA vs ICN (80 to 60 pA at 6 DIV, 130 to 60 pA at 10 DIV and 130 to 90 pA at 14 DIV). (F) pCREB immunolabeling, and nuclear counterstaining with DAPI in CA1 of hippocampal organotypic slices cultured on PA, Na⁺-ICN and K⁺-ICN. (G) AnkrynG (axon initial segment marker) and Naᵥ1.6 immunolabeling, and counterstained with DAPI in CA1 of hippocampal organotypic slices cultured on PA, Na⁺-ICN and K⁺-ICN. (H) Percentage of pCREB⁺ cells in the different experimental conditions. Error bars represent SEM, ***p<0.0005. (I) Quantification of mean grey values of pCREB IR panel. Error bars represent SEM, ***p<0.0005. (J) Quantification of mean grey values of Naᵥ1.6 at the AIS from panel. Error bars represent SEM, ***p<0.0005.

FIG. 6. Water suppression ¹H NMR (600 MHz, 298 K, 10% D₂O, PBS, pH=7.4) spectrum of AcVVAAEE peptide.

FIG. 7. Full ¹³C NMR (150 MHz, 298 K, 10% D₂O, PBS, pH=7.4) spectrum of AcVVAAEE peptide.

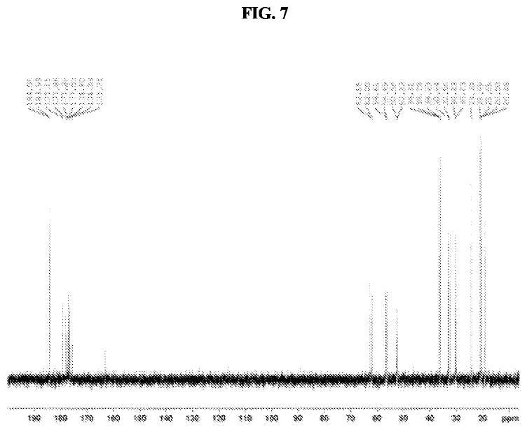

FIGS. 8A-C. ¹H-¹³C 2D HSQC spectrum of AcVVAAEE peptide was acquired with water suppression. (A) Assigned chemical structure with an α- and a side chain; hydrogens for clarity. (B) Region of ¹H—¹³C 2D HSQC spectrum featuring ¹H(α)↔¹³C(α) correlations. (C) Region of ¹H—¹³C 2D HSQC spectrum featuring ¹H↔¹³C correlations from sidechain CH-groups.

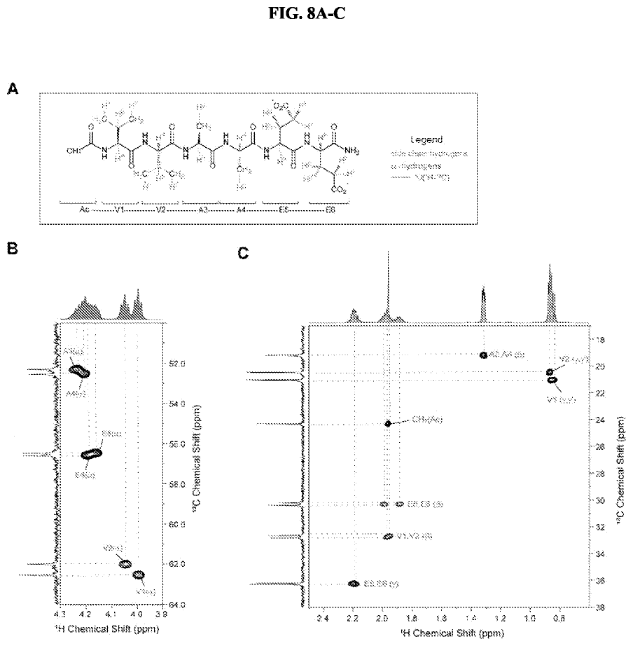

FIG. 9. ¹H—¹³C 2D HMBC spectrum of AcVVAAEE peptide was acquired with water suppression.

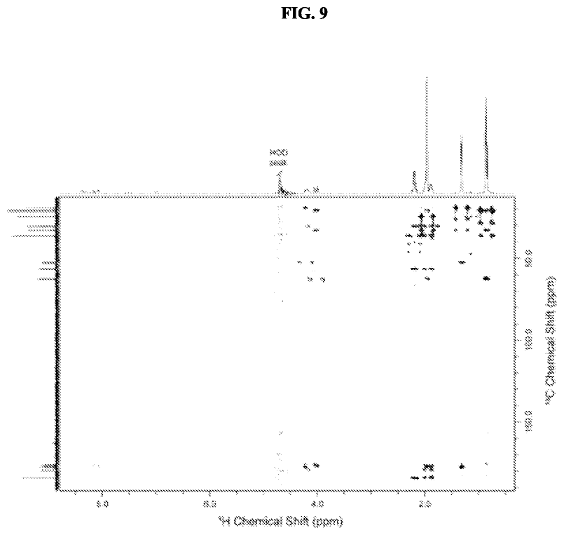

FIGS. 10A-D. Expanded regions of ¹H—¹³C 2D HMBC used for AcVVAAEE peptide residue specific ¹H and ¹³C NMR peak assignment. (A) Identifying the CH₃(Ac)/CO(Ac) HMBC correlation and beginning the backbone walk analysis via α-H(V1)/CO(Ac). (B) region featuring observed amide NH/CO long range ¹H—¹³C couplings. (C) A region of HMBC highlighting α-H/CO^{amide} long range couplings. (D) Diagnostic correlations for E5 and E6 emerging due to long range coupling of Hβ and Hβ′ with CO^{amide} and carboxylates (COO⁻) from E-residucs.

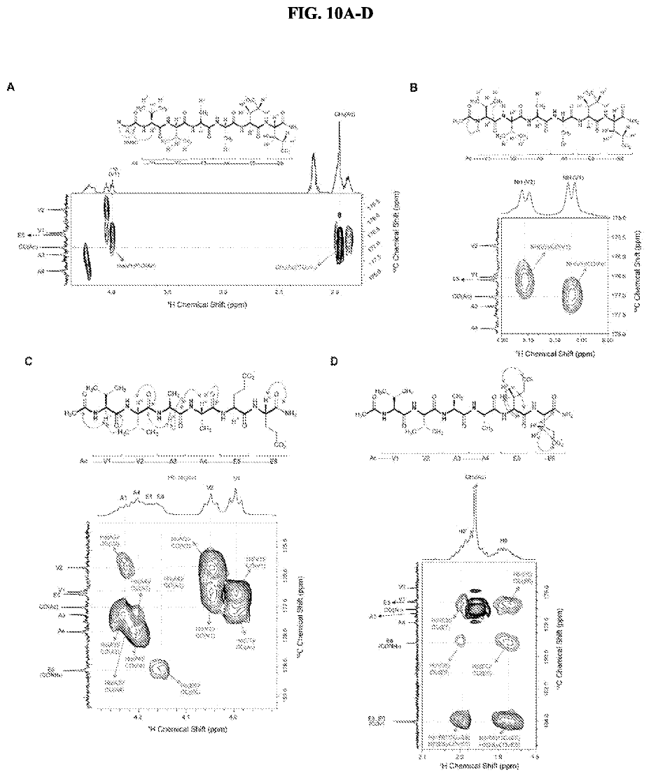

FIGS. 11A-D. ¹H-¹H 2D TOCSY spectra acquired with water suppression 20 and 80 ms mixing times (τₘᵢₓ); τₘᵢₓ=20 ms. (A) Assigned chemical structure with an α-, a side chain, and amide NH protons for clarity. Hartman Hahn magnetization transfer is allowed to evolve within each AA residue. (B) Region of ¹H—¹H TOCSY spectrum featuring correlations of the α-hydrogens with the corresponding side chain protons. (C) Region of ¹H—¹H TOCSY spectrum featuring homonuclear correlations among different side chain protons. (D) Region of ¹H—¹H TOCSY spectrum featuring correlations among amide NH and side chain protons. The experiment allowed for magnetization transfer over 2-3 bonds, longer mixing times allow for the Hartman-Hahn magnetization transfer to occur across the whole spin network. Fully assigned ¹H—¹H TOCSY with τₘᵢₓ=80 ms were shown.

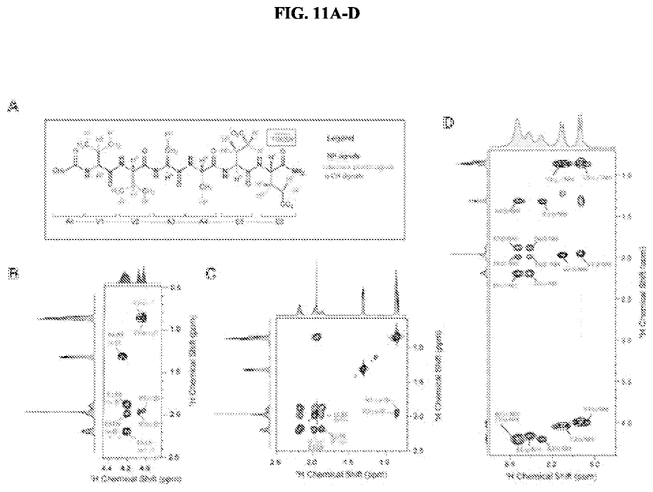

FIG. 12. Assigned ¹H HRMAS NMR (800 MHz, 319 K, MAS 10 kHz) acquired with water suppression on ultracentrifuged PA filaments. E6(α) is attenuated due to water suppression.

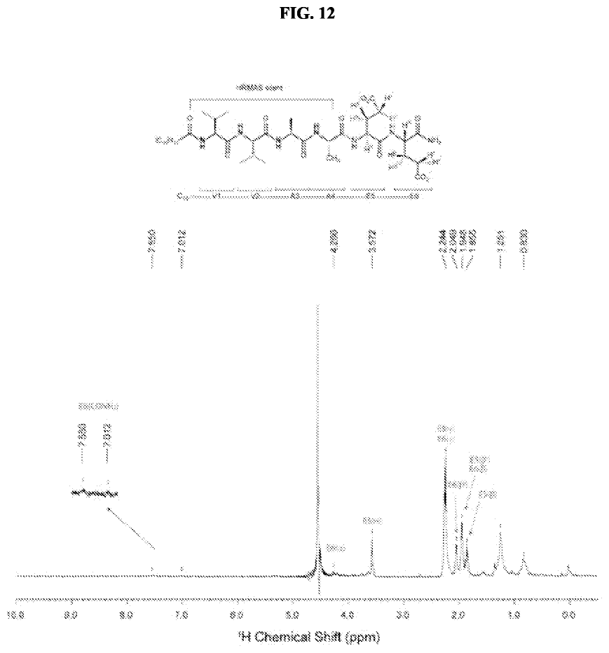

FIG. 13. Assigned ¹H—¹H NOESY NMR (800 MHz, 319 K, MAS 10 kHz, tₘᵢₓ=100 ms) acquired with water suppression on ultracentrifuged PA filaments.

FIG. 14. Assigned ¹H—¹H TOCSY NMR (800 MHz, 319 K, MAS 10 kHz, tₘᵢₓ=80 ms) acquired with water suppression on ultracentrifuged PA filaments.

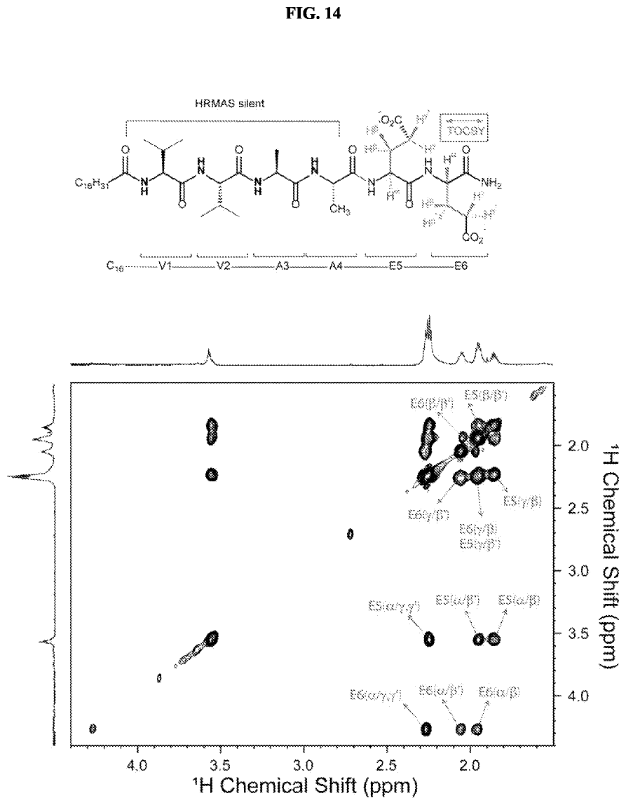

FIG. 15. Assigned ¹H—¹³C HSQC NMR (800 MHz, 319 K, MAS 10 kHz) acquired with water suppression on ultracentrifuged PA filaments.

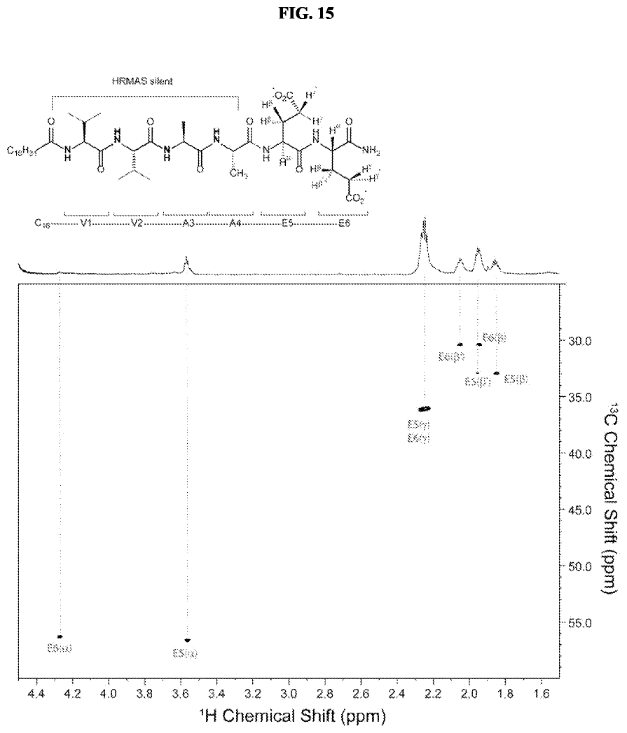

FIG. 16. Assigned ¹H HRMAS NMR (800 MHz, 319 K, MAS 10 kHz) acquired with water suppression on ultracentrifuged PA obtained from 2 mM PA in the presence of 6 molar equiv. of NaOH (double annealing in milliQ and PBS).

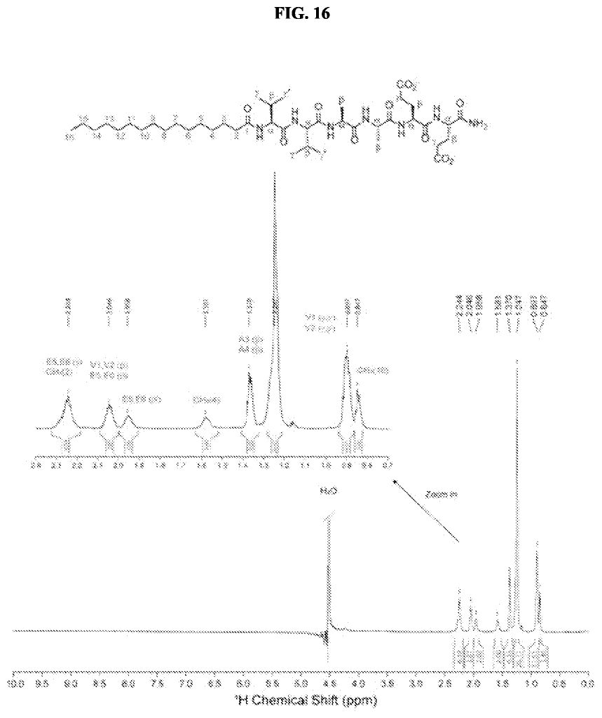

FIG. 17. Assigned ¹H—¹H HRMAS TOCSY NMR (800 MHz, 319 K, MAS 10 kHz) acquired with water suppression on ultracentrifuged PA obtained from 2 mM PA in the presence of 6 molar equiv. of NaOH (double annealing in milliQ and PBS).

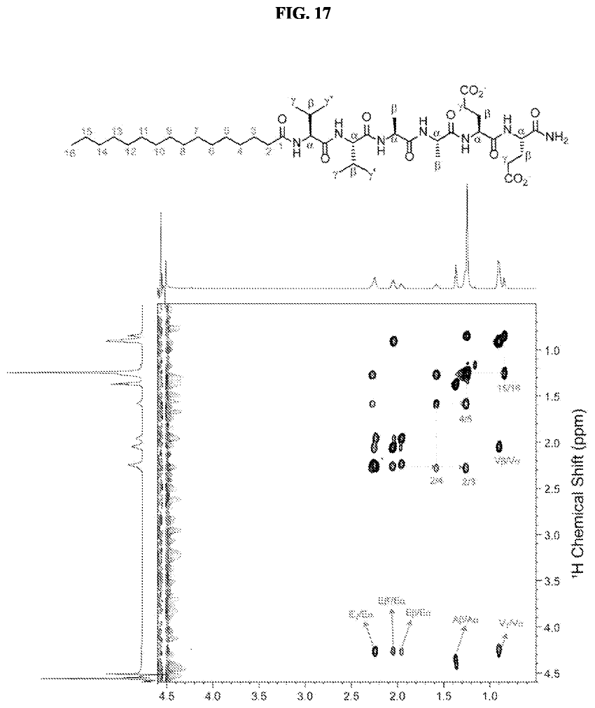

FIG. 18. Assigned ¹H—¹³C HRMAS HSQC NMR (800 MHz, 319 K, MAS 10 kHz) acquired with water suppression on ultracentrifuged PA obtained from 2 mM PA in the presence of 6 molar equiv. of NaOH (double annealing in milliQ and PBS).

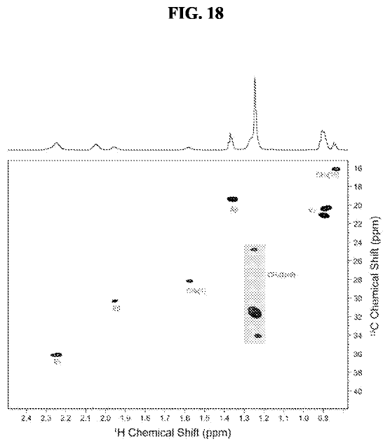

FIGS. 19A-F. (A) Chemical structures of PA and organic cations OC-OC4. Regions of water suppression ¹H NMR spectra (600 MHz, 300 K, 10% D₂O) featuring ¹H signals of organic cations and the corresponding OC-PA assemblies; the spectra are acquired at constant OC concentration and PA/OC ratio (1/16 ratio). (B) Ethyl methyl imidazolium (OC) cation and OC-PA assembly. (C) Ethyl pyridinium (OC1) cation and OC1-PA assembly. (D) N-ethyl-N-methyl pyrrolidinium (OC2) cation and OC2-PA assembly. (E) Trimethylsulfonium (OC3) cation and OC3-PA assembly. (F) Tetramethylammonium (OC4) cation and OC4-PA assembly.

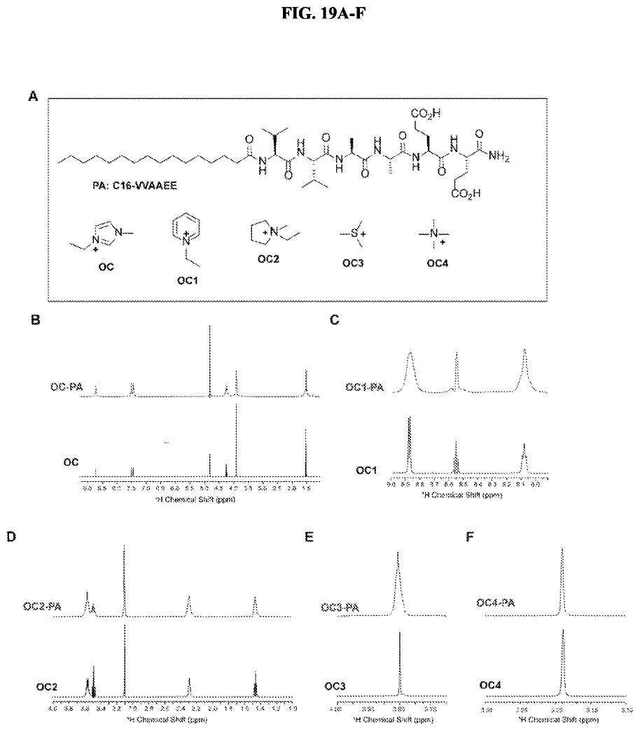

FIGS. 20A-D. Regions of water suppression ¹H NMR spectra featuring the protons of OC. (A) Chemical structure and solution ¹H NMR (600 MHz, 300 K, 10% D₂O) of 160 mM EMIMCl in PBS at pH 7.4 corresponding to the free organic cation (OC_{f}). (B) Solution ¹H NMR (600 MHz, 300 K, 10% D₂O) of OCPA sample prepared with 10 mM PA in the presence of 160 mM EMIMCl in PBS. (C) The ¹H DOSY NMR experiment (600 MHz, 300 K, H₂O/D₂O 9:1, δ=2.20 ms and Δ=105.00 ms) was acquired with water suppression on 160.0 mM EMIMCl in PBS at pH 7.4. The fit of the Hy signal decay to the corresponding mono-exponential Stejskal-Tanner function (plot) returned a diffusion constant value D=(1.179±0.002)·10⁻⁹ m²s⁻¹, corresponding to the free organic cation OC_{f}. The inset provides a focused view of the region of the ¹H DOSY experiment featuring the Hγ-proton. (D) The ¹H DOSY NMR experiment (600 MHz, 300 K, H₂O/D₂O 9:1, δ=2.20 ms and Δ=60.00 ms) was acquired with water suppression on 160.0 mM EMIMCl in PBS at pH 7.4 in the presence of 10.0 mM PA. The inset provides a focused view of the region of the ¹H DOSY experiment featuring the Hγ-proton. The fit of the Hγ signal decay to the corresponding mono-exponential Stejskal-Tanner function (plot) returned a diffusion constant value D=(2.900±0.023)·10⁻¹⁰ m²s⁻¹. The equation used I=I₀exp[−qD] form of Eq. 1 where q=γ²g²δ²σ² (Δ−δ/3) to obtain the fits shown in panels C and D.

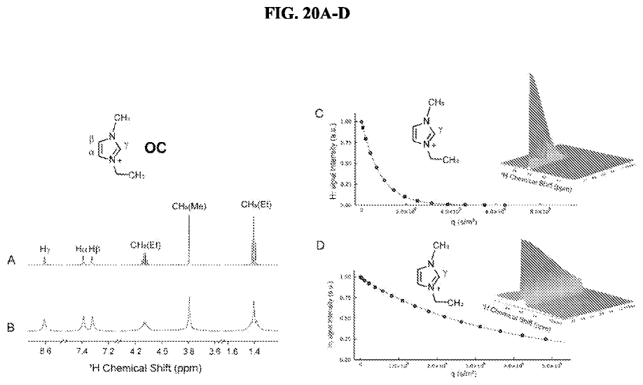

FIG. 21. Quantitative ¹H NMR with water suppression (600 MHz, 300 K, 10% D₂O, d₁=10 s) was acquired on supernatants obtained by ultracentrifugation of OC-PA samples made at 10 mM PA in the presence of varying amounts of OC. Molar equivalents of OC, relative to PA, are given on the right.

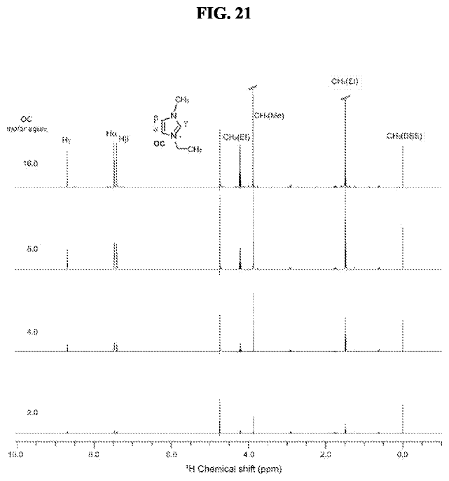

FIGS. 22A-B. (A) An overlay of ¹H HRMAS NMR (600 MHz, 319 K, 10 KHz MAS, 10% D₂O) spectra acquired on pellets obtained by ultracentrifugation of OC-PA samples made at 10 mM PA in the presence of varying amounts of OC. Spectra are acquired under identical conditions and intensities are not scaled in this overlay. (B) Stack of the same ¹H HRMAS NMR spectra shown in panel A where the intensities are scaled to feature an increase of broad resonances.

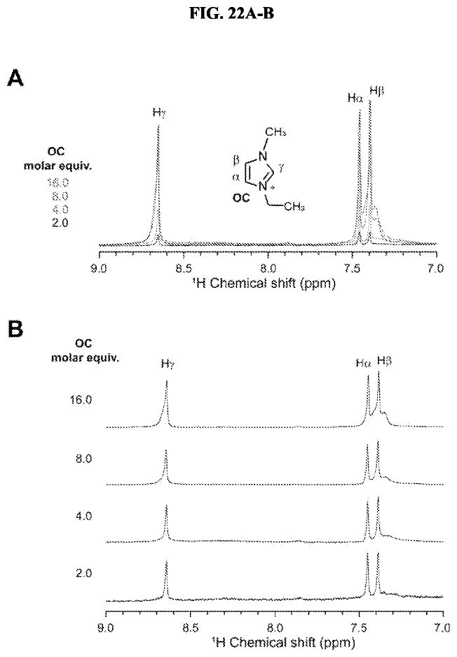

FIG. 23. Assigned ¹H HRMAS NMR (800 MHz, 319 K, 10 KHz MAS, 10% D₂O) spectra acquired on pellets obtained by ultracentrifugation of OC-PA samples made at 10 mM PA in the presence of 16 molar equiv. of OC.

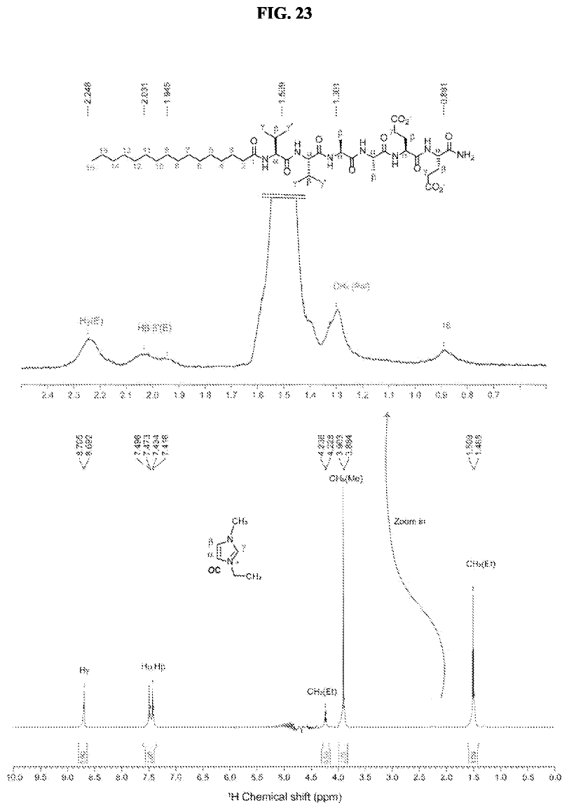

FIGS. 24A-B. (A) ¹H—¹H HRMAS TOCSY and (B) NOESY NMR (800 MHz, 319 K, kHz MAS, 10% D₂O) spectra used for OC ¹H resonance assignment. These are acquired on pellets obtained by ultracentrifugation of OC-PA samples made at 10 mM PA in the presence of 16 molar equiv. of OC.

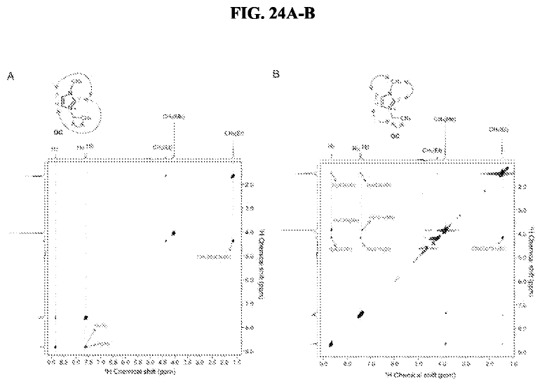

FIG. 25. ¹H T₂ HRMAS NMR (800 MHz, 10 kHz, 319 K, 10% D₂O) of pelleted OCPA assemblies. Shows a representative image of broad signals decay much faster (short T₂) and therefore are assigned to core-bound organic cations (OC_{c}). Sharp peaks decay much more slowly and correspond to free (OC_{f}) and electrostatically attracted OC at the surface of nanostructure (OCₛ), which exchange fast on the NMR timescale.

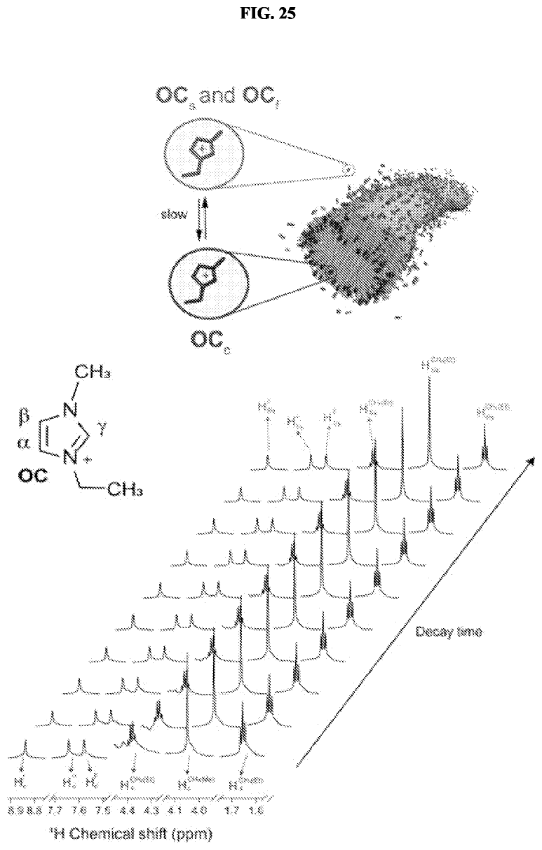

FIGS. 26A-D. (A) ¹H HRMAS NMR (800 MHz, 10 kHz, 319 K, 10% D₂O) of pelleted OCPA assemblies featuring the two sets of peaks observed for OC. Broad signals colored blue are assigned to core-bound organic cations (OC_{c}) while sharp peaks in green correspond to free (OC_{f}) and electrostatically attracted OC at the surface of nanostructure (OCₛ), which exchange fast on the NMR timescale. ¹H HRMAS DOSY NMR (800 MHz, 10 kHz, 319 K, 10% D₂O, D=49.9 ms, d=1.4 ms) was acquired on pelleted OC-PA material using water suppression and exponential gradient ramp. (B) ¹H 2D DOSY plot reveals that slow exchange of ¹H spins on chemical shift and diffusion timescale. (C) A representative fit of the Hy from OC_{c} (blue) and OCₛ and OC_{f} (green signals) signals decay to the corresponding mono-exponential Stejskal-Tanner function returned two diffusion constant values. This was the case for each spin, Hα, Hβ, Hγ, CH₂(Et), CH₃(Me), and CH₃(Et). The diffusion constants and populations calculated as an average of six values for each observed state are: D_{f}=(1.630±0.001)·10⁻¹⁰ m²s⁻¹; pₜ=(81.76±0.05) % (green, fast diffusing) and D_{b}=(3.326±0.004)·10⁻¹² m²s⁻¹ pₜ=(18.24±0.03) % (blue, slow diffusing). (D) A schematic illustration of OC-PA assemblies based on experimental data featuring free and bound OC.

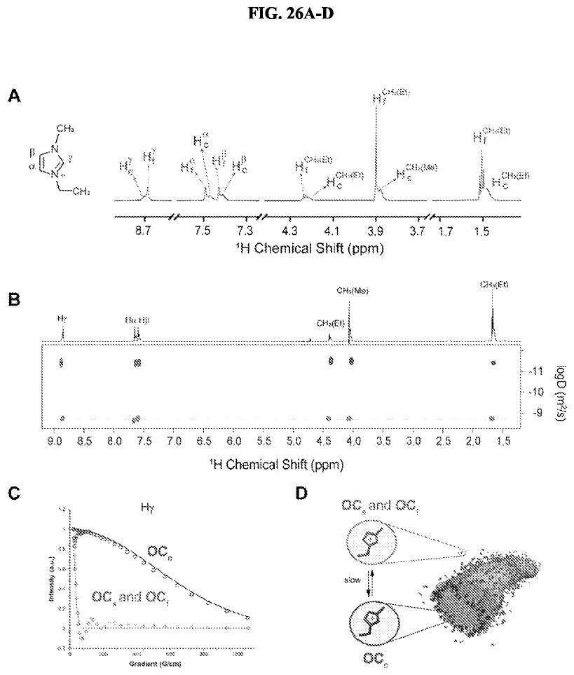

FIGS. 27A-B. (A) Solution ²³Na NMR (159 MHz, 298 K) of PA and OCPA made in PBS. The spectra correspond to PA and OC-PA samples made in PBS at 10 mM PA concentration where total Na⁺ concentration was kept at 145 mM; note that OCPA sample was prepared at PA:OC=1:16. (B) Solution ²³Na NMR (159 MHz, 298 K) of the controls: bottom-control for OC-PA ²³Na relaxation experiments; middle-control for PA ²³Na relaxation experiments; top-control for the experiments shown in subpanel A. All control experiments did not detect any chemical shift changes nor the appearance of satellite peaks.

FIGS. 28A-H. ²³Na longitudinal relaxation rates (R₁) were measured on the (A-D) PA and (E-H) OC-PA (OC/PA=16:1 molar ratio) samples made in PBS containing 14.5 mM of Na⁺ in the presence of varying amounts of K⁺: (A) 1 mM PA and 0.0 mM K⁺, (B) 1 mM PA and 14.5 mM K⁺, (C) 1 mM PA and 43.5 mM K⁺, (D) 1 mM PA and 87.0 mM K⁺, (E) 1 mM OC-PA and 0.0 mM K⁺, (F) 1 mM OC-PA and 14.5 mM K⁺, (G) 1 mM OC-PA and 43.5 mM K⁺, (H) 1 mM OC-PA and 87.0 mM K⁺. Relaxation times used are given on the right side.

FIGS. 29A-B. (A) Fitted ²³Na longitudinal relaxation data shown in Figure SXNa1. (B) Plot showing the dependance of measured ²³Na R₂ for PA-Na⁺ and OCPA-Na⁺ samples as a function of molar equiv. of K⁺ added (molar equiv. of K⁺ are given relative to Na⁺).

FIGS. 30A-H. ²³Na transverse relaxation rates (R₂) were measured on the (A-D) PA and (E-H) OC-PA (OC/PA=16:1 molar ratio) samples made in PBS containing 14.5 mM of Na⁺ in the presence of varying amounts of K⁺: (A) 1 mM PA and 0.0 mM K⁺, (B) 1 mM PA and 14.5 mM K⁺, (C) 1 mM PA and 43.5 mM K⁺, (D) 1 mM PA and 87.0 mM K⁺, (E) 1 mM OC-PA and 0.0 mM K⁺, (F) 1 mM OC-PA and 14.5 mM K⁺, (G) 1 mM OC-PA and 43.5 mM K⁺, (H) 1 mM OC-PA and 87.0 mM K⁺. Relaxation times used are given on the right side.

FIGS. 31A-B. (A) Fitted ²³Na transverse relaxation data shown in Figure SXNa3. (B) Plot showing the dependance of measured ²³Na R₂ for PA-Na⁺ and OCPA-Na⁺ samples as a function of molar equiv. of K⁺ added (molar equiv. of K⁺ are given relative to Na⁺).

FIGS. 32A-D. (A) Simulation setup of a rod-like nanostructure formed by the peptide amphiphile C₁₆A₂V₂E₂ immersed in an aqueous solution containing the organic cation 1-Ethyl-3-methylimidazolium chloride (OC⁺) and sodium chloride. (A) Top view at t=0 showing the nanostructure's cross-section of 8 nm in diameter, approximately. (B) Side at t=0 showing the nanostructure's cylindrical geometry. Chemical structure of the peptide amphiphile (C) C₁₆A₂V₂E₂ and (D) 1-Ethyl-3-methylimidazolium. At t=0, the simulation box dimensions in x-, y-, and z-directions are 16 nm×16 nm×5.5 nm. In the simulation snapshots the color code is as follows: Cl⁻: yellow, Na⁺: light-blue, OC⁺: green.

FIGS. 33A-C. Charge adsorption by the rod-like nanostructure formed by the peptide amphiphile C16A2V2E2. Snapshots of the nanostructure at 200 ns of molecular dynamics simulation in an aqueous solution containing (A) 160 mM, (B) 40 mM, and (C) 0 mM of the organic cation 1-Ethyl-3-methylimidazolium chloride (OC⁺) and sodium chloride. The upper panel is a top view of the simulation box showing the nanostructure's cross-section on the x-y plane. The bottom panel is the side view of the nanostructure composed of two periodic images. The snapshots show that the nanostructure's charge is preferentially compensated by the OC⁺ molecules, which penetrate more into the nanostructure at higher OC⁺ concentrations.

FIGS. 34A-F. Water structure around the nanostructure formed by fully dissociated peptides C₁₆A₂V₂E₂(2−) at the organic cationic [OC]Cl concentrations of (A) 160 mM, (B) 80 mM, (C) 40 mM, (D) 20 mM, (E) 0 mM, and (F) around a partially charged nanostructure of C₁₆A₂V₂E₂²⁻ with no organic cation. The panels show the density profiles of the carbon atoms from C₁₆, the oxygen atoms from water Ow, and the oxygen atoms from glutamic acid OE. Without OC⁺ the water adsorption and intrusion into the nanostructure increases.

FIGS. 35A-F. Distribution of the ionic species around the nanostructure formed by fully dissociated peptides C16A2V2E2(−2) at the organic cation [OC]Cl concentrations of (A) 160 mM, (B) 80 mM, (C) 40 mM, (D) 20 mM, (E) 0 mM and (F) around a partially charged nanostructure of C16A2V2E2⁻ with no organic cation. The panels show OC⁺, Na⁺, and Cl⁻ density profiles in green, blue, and yellow, respectively. At lower OC⁺ concentrations, the OC⁺ adsorption and intrusion into the nanostructure decreases. Cylindrical coordinates system were used (fixed at the center of mass of the rod-like nanostructure) to calculate the density profiles as a function of the cylindrical coordinate, r.

FIGS. 36A-D. Reduced adsorption G of (A) OC⁺ and Na⁺ and (B) water G_{w} in the PA nanostructure as a function of the organic cation concentration in the solution. (C) Mean electrostatic potential profile and (D) water polarization around the PA nanostructure at different organic cation concentrations.

FIGS. 37A-D. (A) Mean square displacement (MSD) of the PA outer glutamic acid when the nanostructure is in an aqueous solution at 160 mM, a zero organic cation concentration and the MSD when the PA has only one glutamic acid dissociated (−1) and at zero organic cation concentration. Snapshots of one PA molecule in the nanostructure in an aqueous solution at (B) 160 mM and (C) 0 mM organic cation concentration, and (D) for the PA in an ionized state (−1).

FIG. 38. Mean square displacement as a function of time of the Na⁺ ions near the fully dissociated PA molecules and in bulk. The average diffusion coefficient in bulk is:

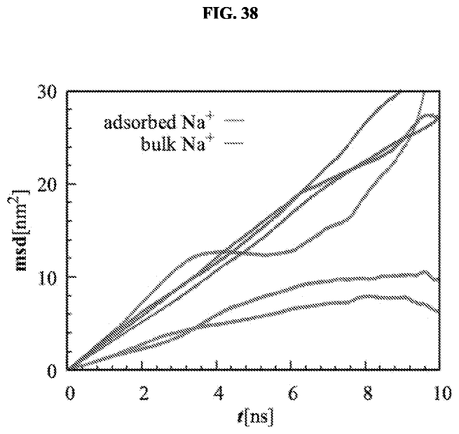

$$D = 0.49 \pm 0.1 \times {10}^{- 5} {cm}^{2} / s$$

and near the nanostructure is:

$$D = 0.052 \pm 0.1 \times {10}^{- 5} {cm}^{2} / s .$$

FIGS. 39A-B. The average degree of dissociation or fraction of charge of the PA-nanofiber (A) and ionic excess per unit length (B) as a function of reservoir pH for different KCl concentrations: The number of PA molecules per unit length is 17.3 nm⁻¹.

FIGS. 40A-B. Ion excess per unit length as function of pH for (A) [KCl]=100 mM and (B) [NaCl]=100 mM. The curve labeled “free” correspond to the ion excess only considering the free ions. The curve labeled “condensed” corresponds to the total number of ions per unit length that are condensed onto the glutamic acids of the PA-nanofiber:

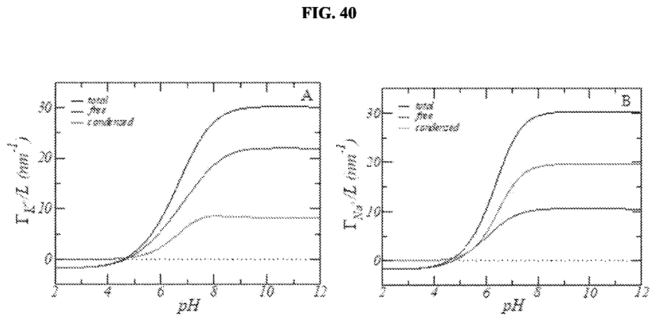

$${\int }_{∞ 0} dr A ( r ) {f}_{AJ} ( r ) 〈 {ρ}_{GLu} ( r ) 〉 .$$

Not only is the speciation, i.e., free vs condensed, different for different ion types, but also the total amount of ion excess (and charge of PA-nanofiber) is ion type specific. The ion excess is found to be ion specific and obeys the following trend: Γ_{Na+}>Γ_{K+}>Γ_{Rb+}>Γ_{Cs+}, which is correlated with the ion size.

FIG. 41. Ion excess per unit length of PA-nanofiber as function of pH for different salts. Salt concentration equals [NaCl]=[KCl]=[RbCl]=[CsCl]=50 mM.

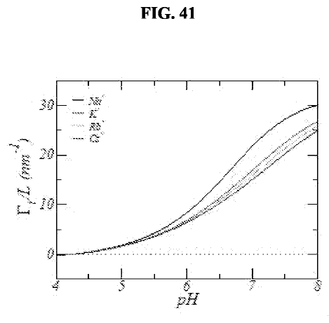

FIG. 42. Extracted non-resonant and cross-terms for 1 mM PA in 66 mM RbCl (Left) and 1 mM PA in 50 mM RbCl and 16 eq OC (Right).

FIG. 43. (A) Schematic of an infinitely thin chiral twisted ribbon of width W and pitch T. (B) A Lego-like model for the twisted bilayer ribbon that is used for calculating the scattered intensity. Each parallelepiped has a width W, length to corresponding to the thickness of the bilayer and height h=1 nm. The relative twist between each parallelepiped is 2p/T. (C) A top view of each parallelepiped along with the discretized model for the Rb⁺ distribution. The Rb⁺ ions are assumed to form a condensed layer in the headgroup section with two charged glutamic acid groups, and a diffuse Rb⁺ distribution away from the bilayer. This diffuse layer is modeled as 12 layers with exponentially decaying Rb⁺ distribution.

FIGS. 44A-F. (A) Simulated intensities that best describe the measured SAXS data for 1 mM PA in 66 mM RbCl. These simulations correspond to the case of ribbons with W=17 nm. Here, 50% of the ionizable amino acid groups were found to be charged. The electron densities of the tail and headgroup regions were r, =322 e/nm³ and h=420 e/nm³. (B) The best-fit Rb⁺ concentration profile. The number of Rb⁺ in the condensed layer were found to be equal to the number of Rb⁺ in the diffuse layer. (C) Estimated average differences, weighted over the non-resonant and the cross-terms, in the simulated vs measured intensities as a function of degree of ionization. The differences are minimized for the case of a=0.5 (A). However, the difference in the quality of match is minimal between the cases of a=0.5 and a=0.6 (D). (E-F) For comparison, the poor quality of match between the data and the simulations for the case of a=0.3 and a=0.9 are shown in (E) and (F), respectively.

FIGS. 45A-D. (A) Simulated intensities that best describe the measured SAXS data for 1 mM PA in 50 mM RbCl+16 mM OC—Cl. These simulations were performed under the constraints that the electron densities for the tail (rₜ) and headgroup regions (rₕ) were identical to the best-match values for the case of PA in 66 mM RbCl (rₜ=322 e/nm³ and rₕ=420 e/nm³). These simulations correspond to the case of ribbons with W=13 nm. The best-match was obtained for the case of 1 Rb⁺/PA. The corresponding electron density profile is shown in (B). (C-D) Simulated intensities that best describe the measured SAXS data for 1 mM PA in 50 mM RbCl+16 mM OC for the cases where the Rb⁺/PA were fixed to (C) 1.5 and (D) 1.8.

FIG. 46. A schematic representation of the Peptide Amphiphile (PA) C16-V2A2E2 and the five ion dopants used in this study (1-Ethyl-3-methylimidazolium chloride, OC; 1-ethylpyridinium chloride OC1; 1-Ethyl-1-methylpyrrolidinium chloride, OC2; tetramethylammonium chloride OC3, trimethyl sulfonium chloride, OC4).

FIGS. 47A-F. Titration of PA with OC. Cryo-TEM micrographs of (A) PA, (B) PA+4eqOC and (C) PA+16eqOC respectively (scale bar: 100 nm). (D) SAXS and (E) WAXS profiles of the PA with 0, 1, 2, 4, and 16eqOC (from bottom to the top) (scattering curves were offset for clarity). (F) Statistical analysis of the Pa and OC-PA for the number of twisted structures, the pitch length, and the width from the cryo-TEM.

FIGS. 48A-B. (A) Scanning electron micrographs of PA (left) and OC-PA (right) coatings, showing less bundling of OC-PA (16 eqv. of OC1) nanostructures compared to PA alone (scale bar: 100 nm). Insets: photographs of PA and OC-PA solutions showing higher turbidity for PA solutions, indicative of more bundling among nanostructures. (B) Size distribution of PA and OC-PA samples measured for electrophoretic mobility testing.

FIG. 49: Cell viability of PAs loaded with various ionic liquids (IDs). Representative fluorescent micrographs and % cell survival quantitation of embryonic mouse cortical neurons seeded on PAs (C₁₆V₂A₂E₂) loaded with various organic cations. PAs were mixed as follows: PA alone; PA+1-Ethyl-3-methyl imidazolium chloride (OC); PA+1-Ethyl-3-methyl imidazolium bromide (OC2); PA+1-Ethyl-3-methyl imidazolium hydrogen sulfate (OC3); PA+1-Ethyl-3-methyl imidazolium bis(trifluoromethylsulfonyl)imide (OC4); PA+tetramethylammonium chloride (OC5). Cells were labeled with cell permeable Calcein (living cells) and propidium iodide (dead cells) for 30 minutes and imaged live using optically clear plates. Scale bar, 50 μm. ***p<0.001, Student's two-tailed t-test.

FIG. 50. Characterization of neuronal culture on PA and OC-PA. Representative fluorescent micrographs of neurons cultured on PA and OC-PA for 1 weeks in vitro. Cells were immunostained for NeuN (nuclear neuronal marker), MAP-2 (neuronal marker), TUJ-1 (neuronal marker), GFAP (astrocytic marker), GABA (GABAergic neuronal marker) and DAPI (nuclei). Scale bars: 25 mm. Bottom line: Dot plot representing the percentage of cells showing expression of both (from left to right) Tuj-1 and DAPI, MAP-2 and Tuj-1, NeuN and Tuj-1, GABA and Tuj-1 markers in neuronal cultures on PA and OC-PA at 2 weeks in vitro. **P<0.001, Student's two-tailed T-test. Right Panel: Growth cone area between PA and OC-PA.

FIGS. 51A-G. Electrophysiological parameters measured in neurons cultured on PA and OC-PA. (A) Plot representing current injection versus number of spikes for neurons cultured on PA and OC-PA, (B) a resistance membrane potential, (C) a input resistance at resting membrane potential. Dot plot graphs representing the (D) fast afterhyperpolarization (f-AHP) amplitude and (E) afterpolarizations (AHP) for conditions referred on J. *P<0.01, **P<0.001 and ***P<0.0001, Student's two-tailed T-test. (F) A violin plot indicating afterpolarization threshold and (G) a dot plot indicating action potential half-width from neurons cultured on PA and OC-PA. *P<0.1 and ***P<0.001, Student's two-tailed T-test.

FIGS. 52A-C. Effect of NBQX on neurons seeded on multi-electrode array (MEA) recordings coated with PA and OC-PA. (A) A schematic of 12-well MEA plate used for the experiment. (B) Representative bright field images of neurons cultured on MEA wells coated with PA and OC-PA and treated with the antagonist of the AMPA receptors, NBQX (OC-PA+NBQX, 5 mM). (C) Activity map of single MEA wells with neurons grown until day 28 in vitro on PA and OC-PA and treated with NBQX (OC-PA+NBQX, 5 mM).

FIG. 53. Nyquist plot from electrochemical impedance spectroscopy (EIS) measurements of PA and OC-PA (16 eqv. of OC1) hydrogel coatings. Coatings were prepared with the same procedure as in vitro studies on glass coverslips and measured in PBS buffer to mimic the cell experiments. The initial semicircle at low impedance values is attributed to the ionic charge transfer resistance of ions hopping along the fibers, while the long tail at low frequencies (high impedance values) is associated with diffusion limited conductivity in the bulk solution. From fitting the semicircles in Autolab NOVA, we found similar ionic charge transfer resistances for PA and OC-PA materials.

FIGS. 54A-J. Coarse-grained Molecular Dynamic Simulation: (A) Radial distribution of PA and OC-PA, water, and Na⁺. (B) OC distribution in PA nanofilaments with total charge per PA molecule=−1. (C) OC distribution in PA nanofilaments with total charge per PA molecule=−2. Water distribution in PA nanofilaments with different equivalents of OC and with (D) −1 and (E) −2 charge respectively. (F) Cl distribution in the nanofilaments upon addition of different equivalents of OC. (G) Na on the surface of PA upon addition of different equivalents of OC and at three different charge density. (H) OC equivalents in the PA nanofillaments upon addition of different equivalents of OC, (I) Cl on the surface of PA upon addition of different equivalents of OC and at three different charge density, (J) Water in the PA nanofilaments upon addition of different equivalents of OC and at three different charge density.

FIGS. 55A-B. pH titration curves for (A) PA and (B) OC-PA with the estimated charge states at different pH values. Each point is from an individual solution that was annealed with the added acid or base prior to measurement.

FIG. 56. Inductively coupled plasma mass spectrometry (ICP-MS) results from the gels prepared from PA and ICN material. Left: Ions which are stayed in the gels upon washing (results for the different monovalent ions: Na, K, Rb and Cs). Right: Ions that released from the gels (Na) upon several washings with milliQ water.

FIGS. 57A-B. (A) Ion release monitored by isothermal titration calorimetry. The ion release process is exothermic with a characteristic enthalpy (DH⁰). Ion doped PAs shows slightly larger heat compared non doped PAs. (B) Recovery curves from fluorescence recovery after photobleaching (FRAP) for PA and ICN with fluorescently labelled C₁₆-V₂A₂E₂K-(Alexa488). The recoveries for the two systems are practically identical, indicating that organic cations do not destabilize or change the dynamics of nanofibers (every curve is the average of three measurements).

FIGS. 58A-C. Cs⁺-ICN is detrimental for mouse embryonic cortical neuron development. (A-a′) Representative fluorescent micrographs and % cell survival quantitation of mouse embryonic mouse cortical neurons seeded on ICNs for 3 DIV and loaded with 125 mM cesium chloride (CsCl); 125 mM sodium chloride (NaCl); or 125 mM potassium chloride (KCl). Cells were labeled with cell permeable Calcein (living cells) and propidium iodide (dead cells) for 30 minutes and imaged live using optically clear plates. Scale bar, 100 μm. ***p<0.001, Student's two-tailed t-test. ns, non-significant. (B-b′) Representative fluorescent micrographs of confocal Z-stacks from mouse embryonic cortical neurons immunolabeled with β-Tubulin 3, and counterstained with DAPI after 3 DIV. Scale bar, 20 μm. Graph denotes quantitation of average neurite length from all conditions at 3 DIV. n=30 neurons per condition. ***p<0.001; ns, non-significant; Student's two-tailed t-test. (C) Representative fluorescent micrographs of confocal Z-stacks from mouse embryonic cortical neurons immunolabeled with b-Tubulin 3, and counterstained with DAPI at 7 DIV. Brackets denote cell clusters of more than 5 nuclei, which were rarely observe in Na⁺-ICN or K⁺-ICN conditions. Scale bar, 50 mm.

FIG. 59. ICNs trigger mitochondrial polarization. Mouse cortical neurons were treated for 6 days with PA control, Na⁺-ICN or K⁺-ICN and immunolabeled with phosphorylated-Itgb, MitoTracker dye, and counterstained with the nuclear marker DAPI. Boxed areas in bottom panels represent single cells shown at top. Scale bars, 5 mm.

FIGS. 60A-C. K⁺-ICN upregulates pCREB protein levels in dendritic compartments. (A) Mouse E16.5 (E, embryonic day) cortical neurons were treated for 6 days with PA control, Na⁺-ICN or K⁺-ICN. Neurons were immunolabeled with β-Tubulin 3 and phosphorylated-CREB at S133, and counterstained with the nuclear marker DAPI. Boxed areas in bottom panels represent cells shown at top. Scale bar, 20 μm. (B) Inhibition of pCREB activation vis forskolin by the commercially available CREB inhibitor 666-15. Mouse cortical neurons were stimulated with forskolin at 100 nM; CREB inhibitor alone at 100 nM; or a combination of both at 100 nM each for 15, 30 and 60 minutes. Immunoblots show expression of pCREB, Total CREB or β-Actin as loading control. (C) Mouse E16.5 (E, embryonic day) cortical neurons were treated for 3 days with PA control, Na⁺-ICN or K⁺-ICN. Neurons were immunolabeled with β-Tubulin 3 and phosphorylated-CREB at S133, and counterstained with the nuclear marker DAPI. Boxed area in bottom panel is enlarged to the right to highlight the expression of pCREB within dendritic compartments (boxed areas).

FIGS. 61A-D. CREB-dependent control of neurite and axon growth in human iPSCs derived to dopaminergic neurons. (A-D) Triple-immunolabeling of pCREB, TH, and human nuclear antigen protein (HuNu) from human dopaminergic neurons derived from iPSCs treated with PA or ICNs plus vehicle or CREB inhibitor (sc-166 at 100 nM) for 3 days. Scale bar, 10 μm. Graph denotes average neurite length quantitation from 30 neurons per condition. ****p<0.0001, *p<0.05. 1-way ANOVA. (C-D) Human dopaminergic neurons derived from iPSCs were treated with PA or ICNs plus vehicle or CREB inhibitor (sc-166) for 6 days and immunolabeled with TH antibodies. Graph denotes average neurite length quantitation from 30 neurons per condition. ****p<0.0001, *p<0.05. Scale bar, 100 μm. 1-way ANOVA.

FIG. 62A-D. ICNs differently modulate intrinsic electrophysiological parameters. (A) Input resistance is increased at 6 DIV but not at 10 and 14 DIV in neurons cultured on Na⁺-ICN. Left, pooled data of single neurons' recordings (Mann-Whitney test *p<0.05). Right, representative traces of the input resistance measured at the steady state (after h-current) through negative current injections (−50 pA). (B) Intrinsic properties measured on single action potentials (amplitude, threshold and afterhyperpolarization). Left, pooled data of single neuron recordings. Amplitude is increased in neurons cultured in Na⁺-ICN at all time points (Mann-Whitney test *p<0.05); threshold of action potential initiation is shifted at more negative values in Na⁺-ICN cultured neurons (Mann-Whitney test *p<0.05; **p<0.005); AHP is not significantly different overtime. (C) Input resistance is increased at all time points in neurons cultured on K⁺-ICN. Left, pooled data of single neurons' recordings (Mann-Whitney test *p<0.05; ***p<0.0005). Right, representative traces of the input resistance measured at the steady state (after h-current) through negative current injections (−50 pA). (D) Intrinsic properties measured on single action potentials (amplitude, threshold and afterhyperpolarization). Left, pooled data of single neurons recordings. Amplitude is increased in neurons cultured in K⁺-ICN at 14 DIV only (Mann-Whitney test *p<0.05); threshold of action potential initiation is shifted at more negative values in K⁺-ICN cultured neurons (Mann-Whitney test *p<0.05); AHP is significant at 6 and 10 DIV, but not at 14 DIV (Mann-Whitney test *p<0.05; **p<0.005).

FIG. 63A-C. (A) Bright-field images of human induced pluripotent stem cell (iPSC)-derived neurons cultured on OC-IKVAV PA, IKVAV PA alone, and laminin for 10 days. Neurites are false colored purple and cell bodies are colored yellow. (B, C) Quantification from bright field imaging of number of cells (B) and neurite length (C). A synergistic effect of the bioactive IKVAV PA co-assembled with the OC (EMIM-Cl) was observed on neurite length.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

The term “amino acid” refers to natural amino acids, unnatural amino acids, and amino acid analogs, all in their D and L stereoisomers, unless otherwise indicated, if their structures allow such stereoisomeric forms. Embodiments herein refer to various amino acid abbreviations (single-letter or three-letter abbreviations) that will be understood by those in the field. Any amino acid abbreviations not defined herein refer to their field-accepted meaning.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyroolysine may also be considered proteinogenic amino acids

The term “non-proteinogenic amino acid” refers to an amino acid that is not naturally-encoded or found in the genetic code, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another: 1) Alanine (A) and Glycine (G); 2) Aspartic acid (D) and Glutamic acid (E); 3) Asparagine (N) and Glutamine (Q); 4) Arginine (R) and Lysine (K); 5) Isoleucine (I), Leucine (L), Methionine (M), and Valine (V); 6) Phenylalanine (F), Tyrosine (Y), and Tryptophan (W); 7) Serine(S) and Threonine (T); and 8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine(S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence, may also be expressed as having a maximum number of substitutions (or terminal deletions) with respect to that reference sequence. For example, a sequence “having at least 70% sequence identity with SEQ ID NO: X” may have up to 3 substitutions relative to SEQ ID NO: X (when SEQ ID NO: X is 10 amino acids in length), and may therefore also be expressed as “having 3 or fewer substitutions relative to SEQ ID NO: X.” Further, a sequence “having at least 80% sequence similarity with SEQ ID NO: X” may have 0, 1, or 2 non-conservative substitutions relative to SEQ ID NO: X, and may therefore also be expressed as “having 2 or fewer non-conservative substitutions relative to SEQ ID NO: X.”

As used herein, the term “nanofiber” refers to an elongated or threadlike filament (e.g., having a significantly greater length dimension that width or diameter) with a diameter typically less than 100 nanometers (e.g., 10 nm).

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, marcomolecules, etc.) and the multicomponent assemblies, complexes, systems, and/or fibers that form as a result.

As used herein, the term “physiological conditions” refers to the range of conditions of temperature, pH and tonicity (or osmolality) normally encountered within tissues in the body of a living human.

As used herein, the terms “self-assemble” and “self-assembly” refer to formation of a discrete, non-random, aggregate structure from component parts; said assembly occurring spontaneously through random movements of the components (e.g., molecules) due only to the inherent chemical or structural properties and attractive forces of those components.

As used herein, the term “peptide amphiphile” refers to a molecule that, at a minimum, includes a non-peptide lipophilic (hydrophobic) segment, a structural peptide segment and optionally a functional peptide segment. The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptidic segment (e.g., comprising an acyl group of six or more carbons), (2) a structural or β-sheet-forming peptide segment; (3) a carboxyl-rich peptide segment, and optionally (4) a bioactive moiety.

As used herein and in the appended claims, the term “lipophilic moiety” or “hydrophobic moiety” refers to the moiety disposed on the N-terminus of the peptide amphiphile (e.g., an acyl moiety), and may be herein and elsewhere referred to as the lipophilic or hydrophobic segment or component. The hydrophobic component should be of a sufficient length to provide amphiphilic behavior and micelle (or nanosphere or nanofiber) formation in water or another polar solvent system. Accordingly, in the context of the embodiments described herein, the hydrophobic component preferably comprises a single, linear acyl chain of the formula: CH₃Cₙ₋₂H₂ₙ₋₂C(O)— where n=6-22. For example, a C16 acyl is of the structure:

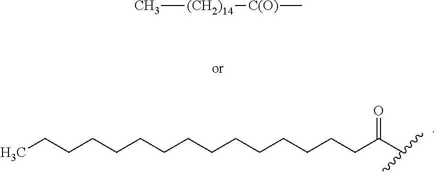

In some embodiments, a linear acyl chain is the lipophilic group, palmitic acid. However, other small lipophilic groups may be used in place of the acyl chain.

As used herein, the term “structural peptide” or “beta-sheet forming peptide” refers to the intermediate amino acid sequence of the peptide amphiphile molecule between the hydrophobic segment and the charged peptide segment of the peptide amphiphile. This “structural peptide” or “beta-sheet forming peptide” is generally composed of three to ten amino acid residues with non-polar, uncharged side chains, selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety). In a preferred embodiment, the N-terminus of the structural peptide segment is covalently attached to the oxygen of the lipophilic segment and the C-terminus of the structural peptide segment is covalently attached to the N-terminus of the charged peptide segment.

As used herein, the terms “carboxy-rich peptide segment” and “acidic peptide segment” refer to the peptide sequence that (i) is linked to the structural peptide segment (beta-sheet forming segment), and/or (ii) is the C-terminal segment of a PA. In some embodiments, the carboxy-rich peptide segment two or more amino acid residues that have side chains displaying carboxylic acid side chains (e.g., Glu (E), Asp (D), or non-natural amino acids). A carboxy-rich peptide segment may optionally contain one or more additional (e.g., non-acidic) amino acid residues. Non-natural amino acid residues with acidic side chains could be used, as will be evident to one ordinarily skilled in the art. There may be from about 2 to about 7 amino acids, and or about 3 or 4 amino acids in this segment.

As used herein, the term “bioactive peptide” refers to amino acid sequences that mediate the action of sequences, molecules, or supramolecular complexes associated therewith. Peptide amphiphiles and structures (e.g., nanofibers) bearing bioactive peptides may exhibit the functionality of the functional peptide.

As used herein, the term “organic cation” refers to a carbon-containing compound having a net positive charge. Examples include an organic amine, an organic quaternary ammonium cation, an organic quaternary phosphonium cation. Examples of the organic amine include an organic amine of 4 to 12 carbon atoms. Among them, ethanolamine, diethanolamine, triethanolamine, isopropanolamine, diisopropanolamine and triisopropanolamine are preferable.

The term “monocation”, as used herein, refers to any cation with a single positive charge, i.e. a cation of formula A⁺ where A is any moiety, for instance a metal atom or an organic moiety. The term “dication”, as used herein, refers to any cation with a double positive charge, i.e. a cation of formula A²⁺ where A is any moiety, for instance a metal atom or an organic moiety. The term “trication”, as used herein, refers to any cation with a triple positive charge, i.e. a cation of formula A³⁺ where A is any moiety, for instance a metal atom or an organic moiety. The term “tetracation”, as used herein, refers to any cation with a quadruple positive charge, i.e. a cation of formula A⁴⁺ where A is any moiety, for instance a metal atom.

As used herein, the term “ion cloud” refers to a layer of charged atoms or molecules around a structure that are ionically interacting with complimentary charged elements of the structure. The “density” of the ion cloud is a measure of the number of charged atoms or molecules interacting with and surrounding the structure. For example, a peptide amphiphile nanofiber having glutamic acids in the charged peptide segment may generate an ion cloud of inorganic cations based on the net negative charge of a terminal glutamic acid. A co-assembly of organic cations and peptide amphiphiles having glutamic acids in the charged peptide segment will generate a denser ion cloud because the organic cations causes a greater net negative charge in the charged peptide segment of the PA.

### DETAILED DESCRIPTION

Provided herein are compositions comprising supercharged supramolecular polymers capable of generating dense ion clouds and methods of use thereof, for example, to enhance neuronal development and membrane excitability. In particular, compositions herein comprise co-assemblies of peptide amphiphiles and organic cations.

Electrostatic interactions underlie biological ion transport mediated via simple uniporters but also sophisticated multidomain protein complexes. Ion channels use numerous cooperative non-covalent interactions to actively and selectively “pump” the ions across cell membranes. This bestows complex organisms with sensitive ionic circuitry which is at the core of neuronal activity, driving the development and function of the brain. Experiments were conducted during development of embodiments herein to develop nanoscale supramolecular assemblies that, among other functionalities, provide an alternative biomimetic strategy to mediate ion flow from the extracellular space.

While exploring the interaction of ions in supramolecular assemblies, it was discovered organic cations (OCs) in negatively charged systems can enable the full ionization of their constituent molecules without destroying their structure (15). High electrostatic repulsion does in fact depolymerize assemblies (16, 17). Typically, when oppositely charged molecules are added, one observes immediate electrostatic gelation into soft glasses or massive bundling of assemblies (18, 19). Infrequent examples have been reported where chaotic gelation is avoided by interfacial phenomena that take the system into a different pathway but are still a result of complementary charge complexation and an overall reduction in charge (20-22). Experiments conducted during development of embodiments herein demonstrate the formation of stable, highly charged assemblies of peptide amphiphiles and organic cations that undergo a morphological transformation to generate thinner and regularly twisted filaments without gelation or aggregation. The high charge of these PA/OC co-assemblies creates a dense cloud of small inorganic cations (e.g., sodium or potassium), a phenomenon that resembles the buffering of inorganic ions by glial cells in the CNS (23). Experiments were conducted during development of embodiments herein to explore the interactions of these PA/OC co-assemblies with neurons, motivated by the potential role of ionic conductivity in their behaviors and lead to the discovery that the highly charged assemblies had a significant effect on both the development of neurons and their membrane excitability.

Without being bounded by theory or limited to a mechanism of action, experiments conducted during development of embodiments herein indicate the following interactions underlying the formation of the PA/OC co-assemblies described herein and the characteristics thereof, including the presence of a dense cloud of inorganic cations surrounding the nanofibers. The experiments demonstrate that the organic cations interact via non-covalent interactions (e.g., hydrophobic interactions, Van der Waals forces, etc.) with the structural peptide segment of the peptide amphiphiles, due to the organic nature (e.g., and partial hydrophobicity) of the organic cations. The embedding of the OCs within the structural layer of the PA nanofiber results in an increase net negative charge to the charged segment of the peptide amphiphiles. The increased net negative charge of the charged segment of the peptide amphiphiles draws small inorganic cations to the surface of the supramolecular PA/OC structure, thereby result in a dense cationic ion cloud around the nanofibers. Counterintuitively, the addition of organic cations into the peptide amphiphile structures results in attraction of inorganic cations near the surface of the PA/OC co-assemblies.

In some embodiments, provided herein are supramolecular co-assemblies of peptide amphiphiles and organic cations. In some embodiments, the net charge of the co-assembly generates a dense ion cloud around the PA/OC nanofibers (e.g., greater charge density surrounding the nanofibers than for PA nanofibers without an OC component). In some embodiments, specific inorganic ions are provided to tailor the characteristics of the system. As described herein, the PA/OC co-assemblies exhibit unique structural and electronic characteristics that provide functionalities not available to other systems.

In some embodiments, the composite materials herein comprise a peptide amphiphile nanofiber component. Peptide amphiphile molecules have been demonstrated to be useful as building blocks to create biomaterials, for example, in regenerative medicine. PAs are designed to self-assemble in aqueous conditions into high-aspect-ratio nanofibers measuring approximately 10 nanometers in diameter and microns in length. Their formation is driven mainly by secondary interactions such as collapse of hydrophobic molecular segments away from an aqueous environment and hydrogen bonding among peptide segments leading to β-sheet secondary structure (Hartgerink, E. Beniash, S. Stupp, Science 2001, 294, 1684; incorporated by reference in their entireties). These supramolecular nanofibers can be designed to display a high surface density of bioactive peptides (when bioactive peptides are present on all or a portion of the PAs within a system capable of diverse functions. Various PA nanofibers have been demonstrated to be useful in repair of the central nervous system and cartilage, neovascularization of ischemic heart tissue, enamel growth, and bone repair, among others (Tysseling-Mattiace et al. Journal of Neuroscience 2008, 28, 3814; Shah et al. Proceedings of the National Academy of Sciences 2010, 107, 3293; Webber et al. Proceedings of the National Academy of Sciences 2011, 108, 13438; Huang et al. Biomaterials 2010, 31, 9202; Mata et al. Biomaterials 2010, 31, 6004; Sargeant et al/Biomaterials 2008, 29, 161; incorporated by reference in their entireties).

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus (or C-terminus) of the peptide, in order to create the lipophilic segment. Synthesis typically starts from the C-terminus, to which amino acids are sequentially added using either a Rink amide resin (resulting in an —NH₂ group at the C-terminus of the peptide after cleavage from the resin), or a Wang resin (resulting in an —OH group at the C-terminus). Accordingly, embodiments described herein encompasses peptide amphiphiles having a C-terminal moiety that may be selected from the group consisting of —H, —OH, —COOH, —CONH₂, and —NH₂.

In some embodiments, peptide amphiphiles comprise a hydrophobic (non-peptide) segment linked to a peptide. In some embodiments, the peptide comprises a structural segment (e.g., hydrogen-bond-forming segment, beta-sheet-forming segment, etc.), and a charged segment (e.g., acidic segment, etc.). In some embodiments, the peptide further comprises linker or spacer segments for adding solubility, flexibility, distance between segments, etc. In some embodiments, peptide amphiphiles comprise a spacer segment (e.g., peptide and/or non-peptide spacer) at the opposite terminus of the peptide from the hydrophobic segment. In some embodiments, the spacer segment comprises peptide and/or non-peptide elements. In some embodiments, the spacer segment comprises one or more active functional groups (e.g., alkene, alkyne, azide, thiol, etc.). In some embodiments, various segments may be connected by linker segments (e.g., peptide (e.g., GG) or non-peptide (e.g., alkyl, OEG, PEG, etc.) linkers).

The lipophilic or hydrophobic segment is typically incorporated at the N- or C-terminus of the peptide after the last amino acid coupling, and is composed of a fatty acid or other acid that is linked to the N- or C-terminal amino acid through an acyl bond. In aqueous solutions, PA molecules self-assemble (e.g., into cylindrical micelles (a.k.a., nanofibers)) that bury the lipophilic segment in their core and display the terminal end of the peptide (e.g., a bioactive peptide, when present) on the surface. The structural peptide undergoes intermolecular hydrogen bonding to form beta sheets that orient parallel to the long axis of the micelle.

In some embodiments, compositions described herein comprise PA building blocks that in turn comprise a hydrophobic segment and a peptide segment. In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between) is covalently coupled to peptide segment (e.g., a peptide comprising a segment having a preference for beta-strand conformations or other supramolecular interactions) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. Peptide segments may comprise natural amino acids, modified amino acids, unnatural amino acids, amino acid analogs, peptidomimetics, or combinations thereof. In some embodiments, peptide segments comprise at least 50% (e.g., 55%, 60%, 65%, 70%, 75%, 80%, 85%, 90%, 95%, 100%, or ranges therebetween) sequence identity or similarity (e.g., conservative or semi-conservative) to one or more of the peptide sequences described herein.

In some embodiments, peptide amphiphiles comprise a charged peptide segment. In some embodiments, peptide amphiphiles comprise an acidic peptide segment. For example, in some embodiments, the acidic peptide comprises one or more (e.g., 1, 2, 3, 4, 5, 6, 7, or more) acidic residues (D and/or E) in sequence. In some embodiments, the acidic peptide segment comprises up to 7 residues in length and comprises at least 50% acidic residues. In some embodiments, an acidic peptide segment comprises (Xa)₁₋₇, wherein each Xa is independently D or E. In some embodiments, an acidic peptide segment comprises EE or EEE.

In some embodiments, peptide amphiphiles comprises a structural and/or beta-sheet-forming segment. In some embodiments, the structural segment is rich in G, H, I, L, F, V, and A residues. In some embodiments, the structural and/or beta-sheet-forming segment comprises an alanine- and valine-rich peptide segment (e.g., AAVV (SEQ ID NO: 1), AAAVVV (SEQ ID NO: 2) VVAA (SEQ ID NO: 3), VVVAAA (SEQ ID NO: 4), AAGG (SEQ ID NO: 5), GGAA (SEQ ID NO: 6), or other combinations of V, G, and A residues, etc.). In some embodiments, the structural and/or beta sheet peptide comprises 4 or more consecutive A, G, and/or V residues, or conservative or semi-conservative substitutions thereto. In some embodiments, the structural and/or beta-sheet forming peptide segment comprises 4 or more consecutive non-polar residues (e.g., glycine (G), alanine (A), valine (V), leucine (L), isoleucine (I), methionine (M)). In some embodiments, the structural and/or beta-sheet forming peptide segment comprises 2-16 amino acids in length and comprises 4 or more (e.g., 2, 3, 4, 5, 6, 7, 8, 9, 10, 11, 12, 13, 14, 15, 16, or ranges there between) non-polar aliphatic residues.

In some embodiments, peptide amphiphiles comprise a non-peptide spacer or linker segment. In some embodiments, the non-peptide spacer or linker segment is located at the opposite terminus of the peptide from the hydrophobic segment. In some embodiments, the spacer or linker segment provides the attachment site for a bioactive group. In some embodiments, the spacer or linker segment provides a reactive group (e.g., alkene, alkyne, azide, thiol, maleimide etc.) for functionalization of the PA. In some embodiments, the spacer or linker is a substantially linear chain of CH₂, O, (CH₂)₂O, O(CH₂)₂, NH, and C═O groups (e.g., CH₂(O(CH₂)₂)₂NH, CH₂(O(CH₂)₂)₂NHCO(CH₂)₂CCH, etc.). In some embodiments, a spacer or linker further comprises additional bioactive groups, substituents, branches, etc.

In some embodiments, a PA may comprise a peptide linker between the segments (e.g., hydrophobic non-peptide segment, structural peptide, charged peptide, bioactive peptide, etc.). In some embodiments, a peptide linker may be flexible or rigid. In some embodiments, a peptide linker may be of any suitable sequence. In some embodiments, a peptide linker comprises one or more glycine residues (e.g., G, GG, GGG, etc.).

Suitable peptide amphiphiles, PA segments, PA nanostructures, and associated reagents and methods are described, for example in U.S. Pat. Nos. 11,066,444; 10,792,327; 10,752,656; 10,738294; 10,689,252; 10,316,432; 10,316,180; 9,926,195; 9,650,421; 9,556,232; 9,517,275; 9,512,404; 9,169,294; 8,940,858; 8,834,840; 8,772,228; 8,748,569; 8,850,923; 8,512,693; 8,450,271; 8,138,140; 8,124,583; 8,114,835; 8,114,834; 8,080,262; 8,063,014; 7,851,445; 7,838,491; 7,745,708; 7,683,025; 7,554,021; 7,544,661; 7,534,761; 7,491,690; 7,452,679; 7,390,526; 7,371,719; 6,890,654; herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural segment comprising or consisting of 4-8 V, G, and A residues; and (c) a charged segment (e.g., comprising EE, EEE, etc.). In some embodiments, a peptide amphiphile further comprises a bioactive peptide head linked to the opposite end of the PA as the hydrophobic tail. Any peptide capable of eliciting a biological response (e.g., binding to a protein) may find use as a bioactive peptide in the embodiments here. In particular, any bioactive peptide that has found use in other PA systems, such as those described in the patent documents and other references incorporated herein, may find use as bioactive peptides in the systems herein. In some embodiments, any PAs within the scope described herein, comprising the components described herein, or within the skill of one in the field, may find use.

In some embodiments, peptide amphiphiles comprise a bioactive moiety. In particular embodiments, a bioactive moiety is the C-terminal or N-terminal most segment of the PA (e.g., opposite end from the hydrophobic tail). In some embodiments, the bioactive moiety is attached to the end of the charged segment (directly or by a linker). In some embodiments, the bioactive moiety is exposed on the surface of an assembled PA structure (e.g., nanofiber). A bioactive moiety is typically a peptide, but is not limited thereto. In some embodiments, a bioactive peptide is a therapeutic peptide. Bioactive peptides and other moieties for achieving functionality will be understood. In some embodiments, bioactive moieties are provided having binding affinity for a target protein of less than 10 μM, less than 100 μM, less than 1 μM, less than 100 nM, less than 10 nM, less than 1 nM, etc.

In some embodiments, the bioactive peptide is a binding peptide capable of binding to a bioactive factor (e.g., protein or other bioactive molecule) that is relevant to the intended purpose of the material. In some embodiments, binding of the bioactive peptide to the bioactive factor results in the bioactive factor being associated with the PA nanofiber.

In some embodiments, the bioactive peptide is a peptide mimetic of a bioactive factor (e.g., protein or other bioactive molecule) that is relevant to the intended purpose of the material. In such embodiments, the bioactive peptide mimics the function of the bioactive factor, rather than recruiting the bioactive factor to the nanofiber. In some embodiments, PAs herein display a bioactive peptide capable of binding to or mimicking a function of a bioactive factor selected from protein/polypeptide agents, such as an enzyme, a receptor, a channel protein, a hormone, a cytokine, a growth factor, and antibody drug.

Suitable bioactive factors include bone morphogenic proteins (e.g., BMP-1, BMP-2, BMP-4, BMP-6, and BMP-7); members of the transforming growth factor beta (TGF-β) superfamily including, but not limited to, TGF-β1, TGF-β2, and TGF-β3; epidermal growth factor (EGF), transforming growth factor-alpha (TGF-α), growth differentiation factors (GDF1, GDF2, GDF3, GDF5, GDF6, GDF7, myostatin/GDF8, GDF9, GDF10, GDF11, and GDF15); human endothelial cell growth factor (ECGF); granulocyte macrophage colony stimulating factor (GM-CSF); nerve growth factor (NGF); vascular endothelial growth factor (VEGF); fibroblast growth factor (FGF); insulin-like growth factor (IGF); cartilage derived morphogenetic protein (CDMP); platelet rich plasma (PRP); platelet derived growth factor (PDGF); insulin growth factor one (IGF-I); or any combinations thereof.

In some embodiments, a combination of PAs with and without bioactive peptides are assembled into a supramolecular structure (e.g., nanofiber). In some embodiments, 1-99% (e.g., 1%, 2%, 5%, 10%, 20%, 30%, 40%, 50%, 60%, 70%, 80%, 90%, 99%, or ranges therebetween) of the PAs in a nanofiber comprise a bioactive peptide. In some embodiments, a peptide amphiphile comprises: (a) a hydrophobic tail comprising an alkyl chain of 8-24 carbons; (b) an alanine-, glycine-, and/or valine-rich peptide segment (e.g., AAVV (SEQ ID NO: 1), AAAVVV (SEQ ID NO: 2) VVAA (SEQ ID NO: 3), VVVAAA (SEQ ID NO: 4), AAGG (SEQ ID NO: 5), GGAA (SEQ ID NO: 6), or other combinations of V, G, and A residues, etc.); and (c) a charged segment (e.g., comprising EE or EEE, etc.). In some embodiments, a bioactive peptide amphiphile comprises: (a) a hydrophobic tail comprising an alkyl chain of 8-24 carbons; (b) an alanine-, glycine-, and/or valine-rich peptide segment (e.g., AAVV (SEQ ID NO: 1), AAAVVV (SEQ ID NO: 2) VVAA (SEQ ID NO: 3), VVVAAA (SEQ ID NO: 4), AAGG (SEQ ID NO: 5), GGAA (SEQ ID NO: 6), or other combinations of V, G, and A residues, etc.); (c) a charged segment (e.g., comprising EE or EEE, etc.), and (d) a bioactive peptide.

In some embodiments, compositions herein comprise organic cations co-assembled with peptide amphiphiles.

In some embodiments, the organic cation is a monocation, dication, trication, or tetracation. In some embodiments, the organic cation is a monocation. In some embodiments, the organic cation is selected from ethyl pyridinium, N-ethyl-N-methyl pyrrolidinium, trimethylsulfonium, tetramethylammonium, methylammonium, formamidinium, aziridiium, cyclopropenium, ethylenediamine, and ethyl methyl imidazolium. In some embodiments, the OC is 1-ethyl-3-methylimidazolium. In some embodiments, the OC is selected from 1-butyl-3-methylimidazolium, 1-propyl-3-methylimidazolium, 1-methyl-3-methylimidazolium, 1-methyl-3-ethylimidazolium, 1-methyl-3-propylimidazolium, 1-methyl-3-butylimidazolium, 1-propyl-3-ethylimidazolium, 1-propyl-3-butylimidazolium, 1-butyl-3-methylimidazolium, 1-butyl-3-ethylimidazolium, 1-butyl-3-propylimidazolium, 1-butyl-3-butylimidazolium, etc.

In some embodiments, the organic cation is a small molecule. In some embodiments, the organic cation has a molecular weight of 500 g/mol or less (e.g., 500 g/mol, 450 g/mol, 400 g/mol, 350 g/mol, 300 g/mol, 250 g/mol, 200 g/mol, or less).

In some embodiments, compositions herein further comprise inorganic cations (e.g., in an ion cloud surrounding the PA/OC assemblies). In some embodiments, the inorganic cations from a dense ion cloud around the supramolecular structures (e.g., co-assembly of PAs and OCs). In some embodiments, the inorganic cations are monoanionic, dianionic, trianionic, or tetraanionic. In some embodiments, the inorganic cations are monoanions. In some embodiments, the inorganic cations present in the aqueous solution (e.g., as a salt or buffer) form the ion cloud as a result of the net charge caused by the structural and electronic effects of packing of the OCs within the PA nanofiber. In some embodiments, specific inorganic cations are supplied in the aqueous solution to create an ion cloud of the desired ionic makeup. In some embodiments, the inorganic cations and the resulting ion cloud comprise one or more of Na⁺, K⁺, Ca²⁺, Mg²⁺, Zn²⁺, Sc³⁺, Mn²⁺, Rb⁺, Cs⁺, etc. In some embodiments, inorganic cations are introduced to the PA/OC co-assemblies via salts (e.g., salts of the desired inorganic cation and a suitable anion (e.g., Cl⁻, OH⁻, I⁻, F⁻, etc.) in the aqueous solution the systems are formed in, reconstituted in, stored in etc. In some embodiments, the ion cloud of the PA/OC co-assembly is denser that the ion cloud of the corresponding PA assembly in the absence of OC.

In some embodiments, co-assembly of peptide amphiphiles with organic cations results in the formation of a supercharged nanofiber structure, having a greater proportion of the ionizable amino acids (e.g., amino acids of the charged peptide segment) deprotonated when compared to a peptide amphiphile nanostructure of the same PAs in the absence of organic cations under the same conditions. For example, under neutral conditions (e.g., pH 7.0), typically only the exterior ionizable amino acid of the charged segment with be deprotonated within a nanofiber structure (e.g., C₁₆-AAGGEE⁻); however, in certain embodiments, when the same PA is co-assembled with a suitable organic cation, a higher proportion of the PAs within the nanofiber structure exhibit a greater number of ionizable amino acids deprotonated (e.g., C₁₆-AAGGE⁻E⁻). In some embodiments, a higher percentage (e.g., 50%, 60%, 70%, 80%, 90%, 95%, 99%, 100%) of amino acids in charged segments comprising two ionizable amino acids exhibit both amino acids deprotonated (e.g., E⁻E⁻, E⁻E⁻E⁻, etc.) when compared to nanostructures of PAs alone under the same conditions. In some embodiments, a higher percentage (e.g., 50%, 60%, 70%, 80%, 90%, 95%, 99%, 100%) of amino acids in charged segments comprising three or more ionizable amino acids exhibit two or more amino acids deprotonated (e.g., E⁻E⁻, E⁻E⁻E⁻, etc.) when compared to nanostructures of PAs alone under the same conditions.

In some embodiments, excess organic cation is required relative to peptide amphiphile molecules to achieve a supercharged states. In some embodiments, the more ionizable amino acids present in the PA (e.g., in the charged segment, near the exposed terminus, etc.) the greater the excess of OC required to generate s supercharged supramolecular structure. For example, for an exemplary PA comprising C₁₆-AAGGEE, a molar excess of 10-20 fold may result in a supercharged nanofiber under particular conditions. In other embodiments (e.g., other conditions, other PA, other OC), 2-50 fold (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or ranges therebetween). In some embodiments, equimolar PA and OC achieves a desired supercharged state.

In some embodiments, compositions are provided herein comprising co-assembly of OC and PA in a molar ration that is insufficient to achieve a fully supercharged state. In some embodiments, PA is in excess over OC.

In some embodiments, provided herein are methods of assembling the compositions described herein. In some embodiments, peptide amphiphiles are synthesized using standard techniques understood in the field, for example, fluorenylmethyloxycarbonyl (fmoc) procedures. In some embodiments, supramolecular PA nanofibers are assembled using well-known techniques, such as by placing the PA monomers in aqueous solution and adjusting conditions (e.g., salt concentration, pH, temperature, etc.) to facilitate nanofiber formation. In some embodiments, organic cations are added to the PAs is solution. In some embodiments, the OCs and PAs are combined at an elevated temperature (e.g., 50° C., 55° C., 60° C., 65° C., 70° C., 75° C., 80° C., 85° C., 90° C., or ranges therebetween). In some embodiments, the OCs and PAs are mixed and/or sonicated in solution. In some embodiments, the solution comprising OCs and PAs are allowed to cool to room temperature (e.g., at a rate of 0.5-5° C. per minute (e.g., 0.5° C./min, 1° C./min, 2° C./min, 3° C./min, 4° C./min, 5° C./min). In some embodiments, inorganic ions are added to the assembled OCs and PAs in the form of chloride and hydroxy salts (or other suitable salts), such as NaCl/NaOH, KCl/KOH, CsCl/CsOH, and RbCl/RbOH, with adjustment of pH (e.g., pH adjustment to 6.6, 6.7, 6.8, 6.9, 7.0, 7.1, 7.2, 7.3, 7.4, 7.5, 7.6, 7.7, 7.8, or ranges therebetween). In some embodiments, compositions are lyophilized and reconstituted between any suitable steps.

In some embodiments, the compositions herein find use in enhancing the growth, development, and membrane excitability of neuronal cells (e.g., neurons, astrocytes, microglia, etc.) (e.g., in vitro). In some embodiments, the compositions herein find use in enhancing the growth, development, and membrane excitability of muscle cells or other cell types that respond to action potentials. In some embodiments, the supramolecular co-assemblies herein are provided with neuronal (or other) cells in culture. In some embodiments, systems are provided comprising the PA/OC compositions herein and neuronal (or other) cells. In some embodiments, surfaces (e.g., plastic, glass, metal, etc.) are provided that are coated in the PA/OC compositions herein for the growth of neurons thereon. In some embodiments, neuronal cell growth media is provided with the PA/OC compositions therein. In some embodiments, PA/OC compositions are applied directly to tissues, cells, etc. In some embodiments, any suitable neuronal (or other) cells may find use in the systems and methods described herein. For example, in some embodiments, the PA/OC compositions herein find use in enhancing the growth, development, and membrane excitability of cells derived from stem cells (e.g., induced pluripotent stem cells).

### EXPERIMENTAL

### Example 1

### Dense Ion Clouds Attracted by Supercharged Supramolecular Polymers Boost Development of Neurons

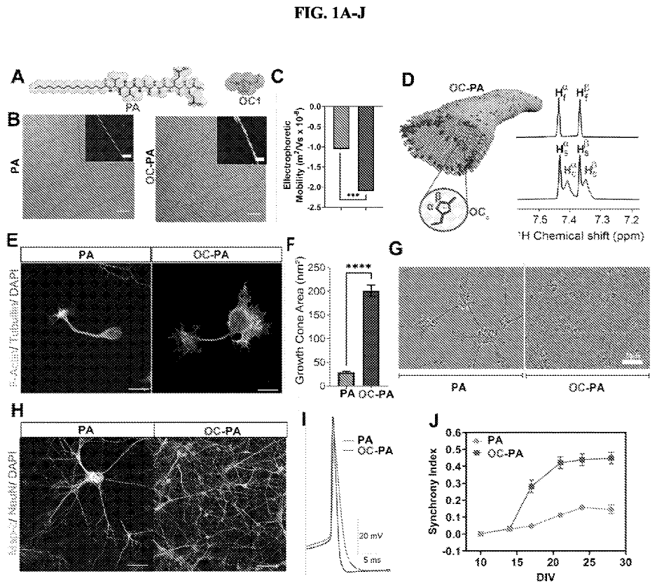

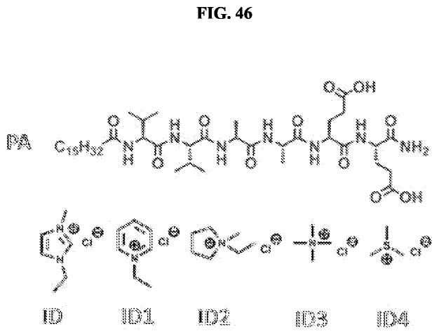

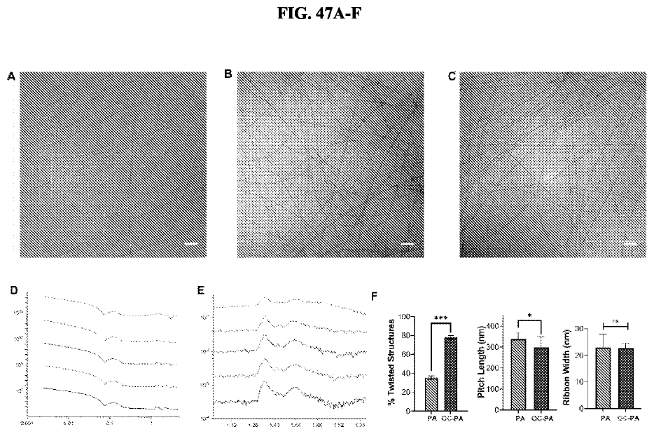

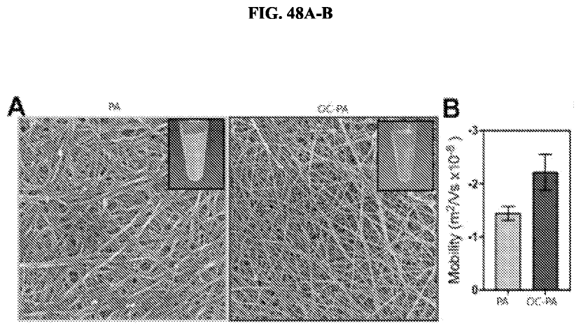

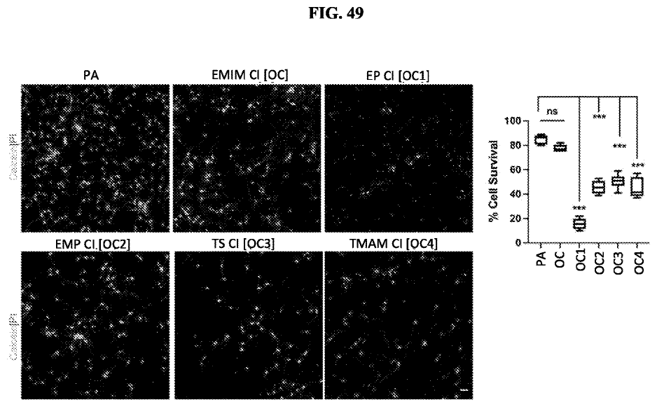

To introduce OCs into supramolecular assemblies, the monomer C₁₆-VVAAEE peptide amphiphile [(PA), (C₁₆=palmitoyl; FIG. 1A)], was chosen as it assembles into well-defined one-dimensional nanostructures. Five different small, hydrophobic OCs were selected given their amphiphilic nature were likely to interact with peptide sequences (FIG. 46, samples with OC are OC-PA from now on). Transmission electron microscopy (TEM) and atomic force microscopy (AFM) revealed a narrower, twisted morphology following the addition of 16 equivalents of the cations (FIG. 1B and FIG. 46,), suggesting their internalization within the nanostructures and significant interactions with peptide molecules. Small-angle X-ray scattering (SAXS) of solutions corroborated the formation of narrower ribbons as evidenced by a decrease in the ln[I(q)] vs ln[q] slope at low q values. Their narrow and highly twisted nature suggested a higher charge relative to the pure PA assemblies. To enhance electrophoretic mobility of assemblies the incorporated OCₛ were compared and measured to PA alone, indicating a higher charge density of OC-PA structures (FIG. 1C and FIG. 47). It was also observed that an absence of nanostructure bundling phenomena in OC-PA samples in scanning electron micrographs and optical density measurements (FIG. 47), consistent with a higher electrostatic repulsion among nanofibers in the OC-PA system. The cation-PA interactions were confirmed by the line broadening observed in solution nuclear magnetic resonance (NMR) spectra (FIG. 48). For that purpose, high-resolution magic-angle spinning (HR-MAS) ¹H NMR spectra measured on centrifuged pellets of the OC-PA assemblies revealed two sets of resonances corresponding to OC1 (FIG. 1D). Since the solution ¹H NMR spectra of the supernatant liquid after centrifugation reveal a single set of resonances for OC1 (FIG. 1D), it was hypothesized that the doubling of peaks results in two different chemical environments for bound OC1 (Index 1: for distinct chemical shifts, relaxation properties, and translational diffusion). However, Fourier-transform infrared (FTIR) spectra and wide-angle X-ray scattering (WAXS) of solutions indicated that the interacting cations do not disrupt the well-known internal b-sheet structure of these PA assemblies (FIG. 49). It was then concluded from these experiments that highly charged PA assemblies had formed by introducing OCs within their structures.

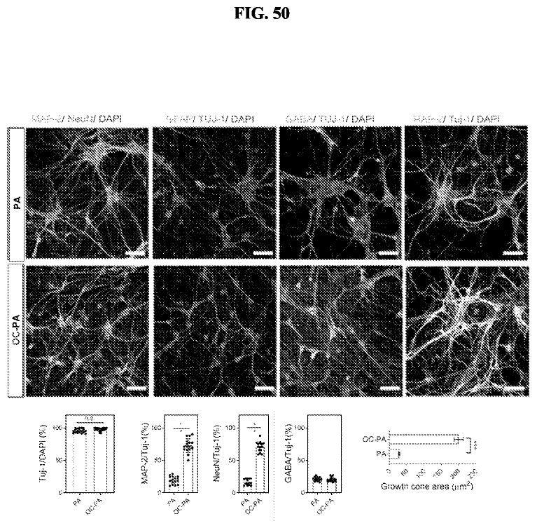

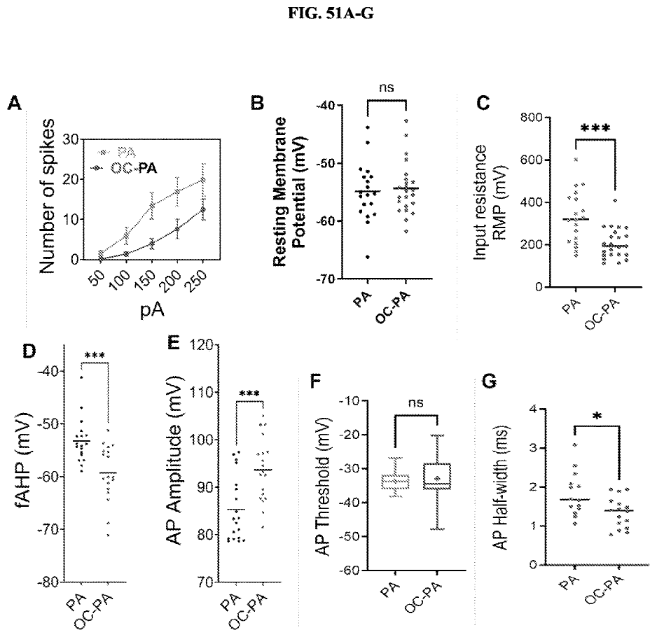

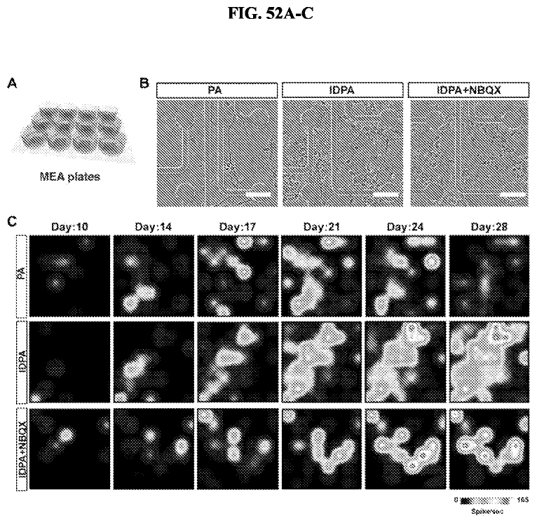

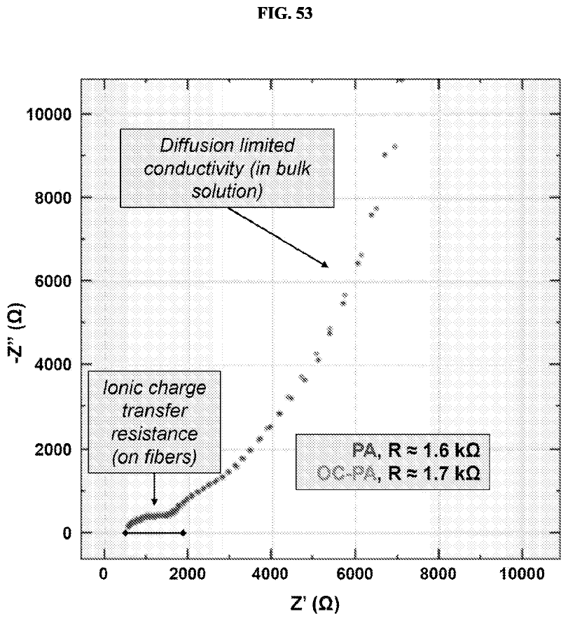

Next, the viability of mouse cortical neurons in contact with all five OC-containing PA systems in saline was tested. As a result, the findings showed that the one with 1-ethyl-3-methylimidazolium (OC1) maintained even greater viability of neurons relative to the pure PA nanostructures (FIG. 50). The other four OC-containing PA nanostructures revealed lower viability thus, the focus was shifted onto the system with OC1 (OC-PA) in subsequent experiments. Additionally, it was also found that in OC-PA striking differences in cell attachment and area of neuronal growth cones relative to PA alone was within the first 24 hours of cell culture (FIG. 1E-F). After a week, neurons grew homogenously on complex neural networks on OC-PA substrates, while those cultured on pure PA tended to aggregate into large clusters (FIG. 1G). Based on the immunocytochemistry and western blots, it was observed that enhanced expression of the neuronal maturation markers MAP-2 and NeuN as well as the post-synaptic marker PSD95 on OC-PA coatings (FIG. 1H-I, and FIG. 51-52). Next, the effect of the different coatings on neuronal function using manual whole-cell patch clamp recordings were assessed. Previously, within the field, it has been established that young neurons have shorter and wider action potentials with slower repolarization due to limited ion channel expression. Neurons cultured on OC-PA exhibited larger action potential amplitudes and repolarization with larger fast afterhyperpolarization (fAHP) compared to those cultured on PA, indicative of enhanced maturity (FIG. 1I and FIG. 53). Using a multi-electrode array platform, which allows for the recording of spontaneous electrical activity of cellular populations, it was observed that the greater synchrony of neuronal firing was relative to those plated-on PA alone (FIG. 1J). These initial results on bioactivity indicated that the high levels of charge in OC-PA nanostructures led to an enhanced level of functional maturation and network formation among cultured neurons.

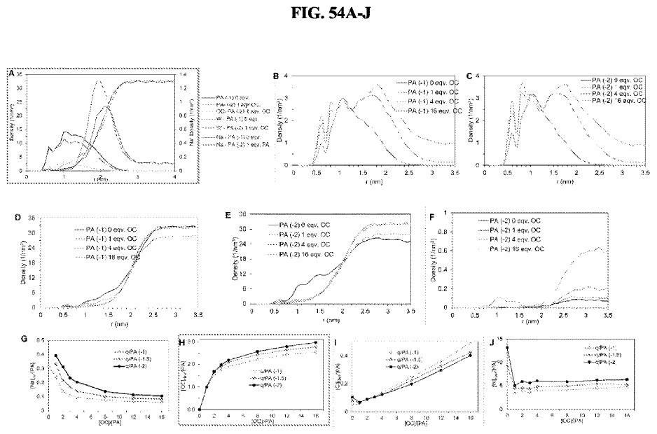

Given the nature of the biological observations, the greater electrical conductivity in these systems was further explored and found to be the explanation for this effect. Based on previous reports linking higher conductivity to enhanced maturation of electrogenic cells such as neurons and cardiomyocytes electrochemical impedance spectroscopy and chronoamperometry were utilized to measure electronic and ionic conductivity in both PA and OC-PA assemblies (FIG. 54). These measurements indicated that there effectively were no differences in conductivity between PA and OC-PA nanostructures. Seeking an explanation for these biological observations, the focus was then turned to more in-depth NMR measurements to explore what might be the physical origin of the elevated charge in OC-PA assemblies mentioned previously.

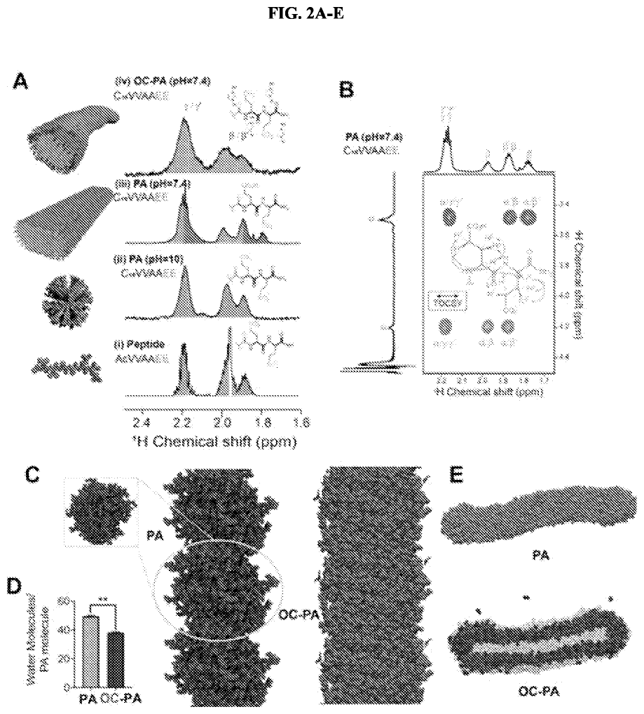

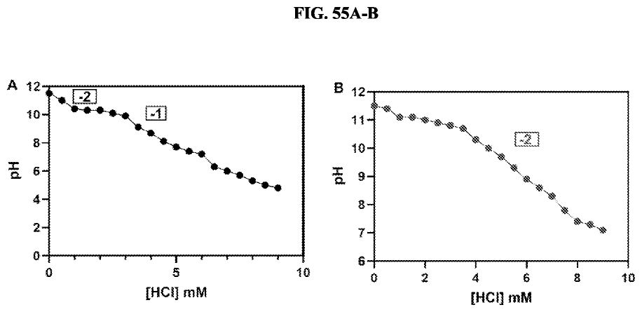

In this regard, it was counterintuitive to us that addition of an organic cation to a negatively charged assembly would further increase rather than decrease the charge density. HR-MAS NMR data indeed confirmed that OC-PA assemblies only contain molecules with fully ionized E residues (FIG. 2A). This is supported by the observation of a single set of resonances by HR-MAS corresponding to side chains of E residues in OC-PA fibers, compared to two sets of resonances observed in fibers containing only PA molecules (i.e., protonated, and deprotonated side chains). In pure PA assemblies at high pH, where the initial expectation was full ionization of E residues and thus formation of spherical micelles (FIG. 55), it was observed that similar spectral features corresponded to OC-PA fibers (FIG. 2A, B). This analysis demonstrated the formation of stable fibrillar assemblies containing fully charged PA molecules when combined with OC1, which is consistent with our morphological characterization, electrophoretic mobility, and optical density measurements.

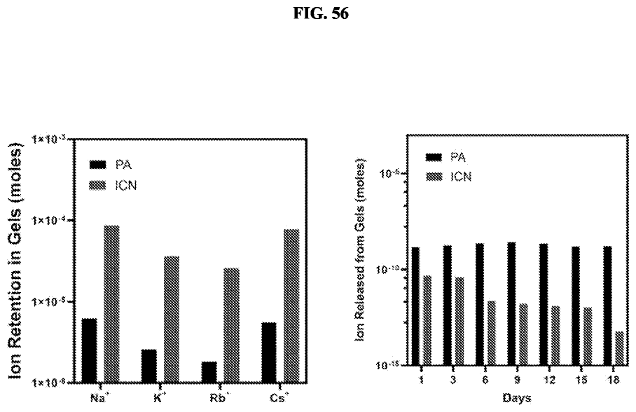

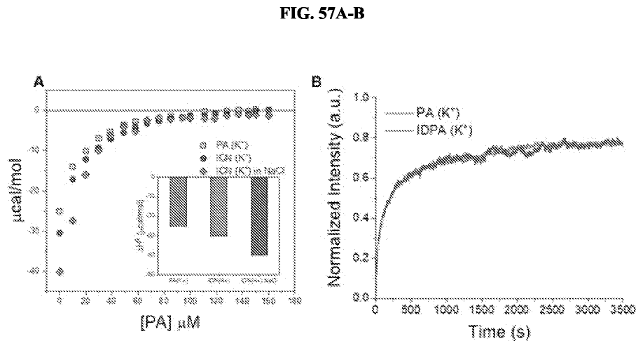

To evaluate possible interactions between PA molecules and OC1, both coarse-grained and atomistic molecular dynamics (MD) simulations were utilized. The simulations supported our experimental observation that PA molecules only assembled into fibrillar structures when their E residues were partially ionized, whereas fully ionized ones spontaneously disassembled into small aggregates (FIG. 2C, left structure). The simulations did predict, in agreement with experimental results, that stable fibers could form with fully ionized molecules upon addition of OC1 (FIG. 2C, right structure). The simulations also indicated that a population of the OC molecules (approximately 3 equiv.) occupies the space between b-sheets by displacing structural water molecules to the periphery of the fiber (FIG. 2D). Furthermore, the predicted location of the organic cations near the b-sheet region rather than near the charged E residues shows that the absence of electrostatic compensation allowed for the enhanced surface charge (FIG. 56). The decrease in the pKₐ was then measured from pH titration assays upon the addition of OC to the PA assemblies (FIG. 57). Thus, the addition of the OC did not cancel the negative charge but, instead induced more negative charge by ionizing both outer and inner glutamic acids. Overall, these results were consistent with the NMR results, which revealed full ionization of all E residues in the OC-PA nanostructures.

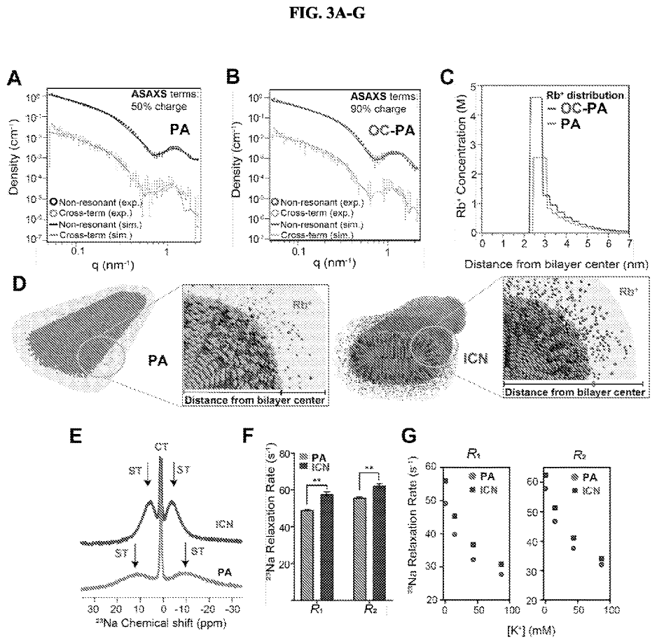

As a result of the absence of conductivity differences between PA and OC-PA, it was then found necessary to further explore if the elevated charge affected the distribution of biologically relevant inorganic cations such as sodium and potassium. For this purpose, the synchrotron anomalous small-angle X-ray scattering (ASAXS) was utilized to directly measure the ion distribution in and around the assemblies using rubidium (Rb⁺) as a proxy for the monovalent cations. ASAXS has been used previously investigated for the counterion distribution around highly charged macromolecules such as DNA and polymer brushes. In these experiments. It was observed that Rb-dependent scattering occurred in both PA and OC-PA samples (FIG. 3A, B), indicating the presence of a counterion layer surrounding these assemblies. The ASAXS scattering profiles with RbCl were similar to the SAXS profiles with NaCl, indicating that there were no major changes in the structure by replacing Na⁺ with Rb⁺, thus validating the approach. Following extraction of the scattering term that is dependent on the Rb contribution, the PA and OC-PA profiles were then fitted to a twisted ribbon geometrical model. As a result, the best-fit models indicated that OC-PA assemblies had nearly twice as many Rb⁺ ions associated with them relative to the PA-only nanostructures (FIG. 3C). Furthermore, in both assemblies the geometrical models revealed that Rb⁺ is absent from their cores and is concentrated in a layer of similar thickness around the charged E residues on the surface of the fibers. These observations indicate a significantly denser “ion cloud” around the surface of nanostructures containing OCₛ (FIG. 3D), which is referred to as ion cloud nanostructures (ICNs) (FIG. 3D). Inductively coupled plasma mass spectrometry (ICP-MS) corroborated the presence of more monovalent ions condensed in the ICNs (FIG. 57).

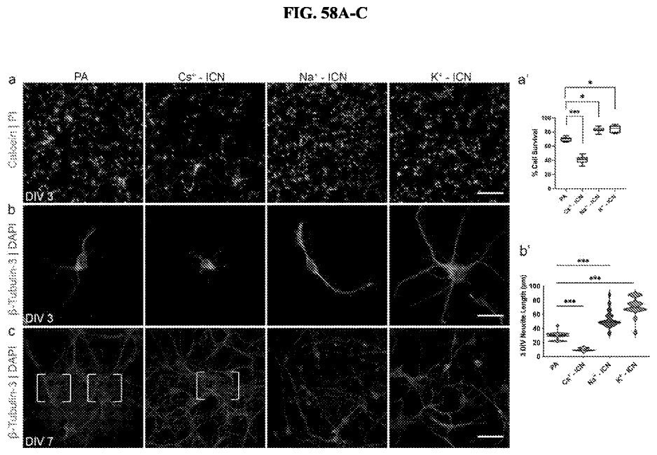

To better understand the origin of these high local concentrations of monovalent cations, the ²³Na NMR spectroscopy solution was utilized, which has been shown to give an isochronous central transition (CT) peak as well as satellite transitions (STs) caused by residual ²³Na quadrupolar coupling when interactions occur between Na⁺ ions and oppositely charged structures such as proteins and polymers. It was found that STs in ICN samples were sharper and less separated from the CT relative to PA samples, which were attributed to the presence of an enhanced electrostatic potential experienced by Na⁺ ions within the cloud of ICN assemblies (FIG. 3E). These sharper ST peaks in ICNs indicate a higher degree of interaction and ordering of Na⁺ ions near the assembly surface, where ASAXS revealed a higher concentration of monovalent ions. Furthermore, the narrower ST linewidth for ICN samples indicates the presence of less dynamic Na⁺ ions. Next, the longitudinal (R₁) and transverse (R₂) ²³Na relaxation rates were measured for PA and ICN samples prepared at the same total Na⁺ concentration and constant Na⁺/PA ratio. Consistent with the suggestion above, ICN assemblies showed higher ²³Na relaxation rates relative to PA nanostructures (FIG. 3F), which indicates more binding of Na⁺ cations within the cloud. Next, investigated was the potential for other biologically relevant monovalent ions like potassium and if it could form an ICN. Without directly measuring the binding of potassium ions directly by NMR, ²³Na NMR relaxometry in a Na⁺/K⁺ competitive binding assay was utilized (FIG. 3G, FIG. 58) by incremental addition of KCl to each sample. These relaxometry measurements revealed the gradual liberation of Na⁺ ions into solution as more KCl was added and K⁺ ions became condensed in the cloud, as indicated by a reduction in ²³Na R₁ and R₂. It was concluded that OC interactions with PA assemblies promote enhanced levels of charge density without disrupting their cohesive supramolecular structure. This in turn enables a unique state of monovalent ion condensation that must be connected to the biological activity discovered here in the maturation of neurons.

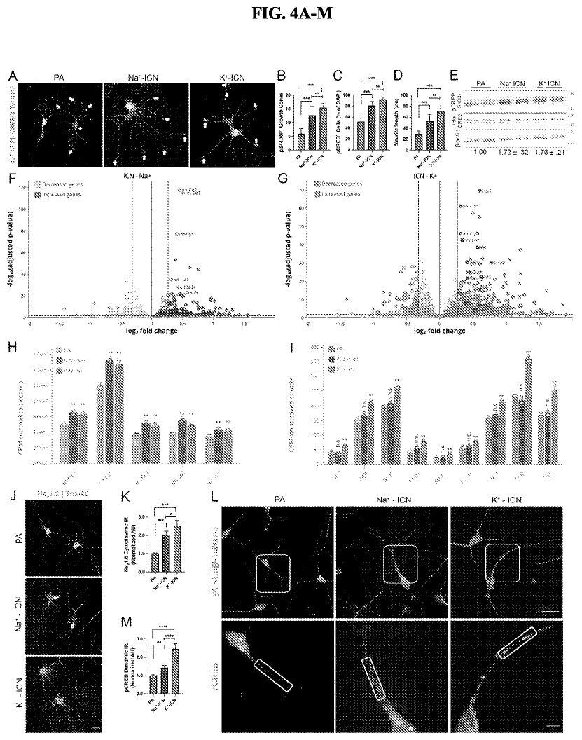

Since the initial biological experiments utilized ICNs prepared in saline which contained both sodium and potassium ions, the specific effects of ICNs prepared exclusively with sodium or potassium ions were further explored. First, mouse embryonic cortical neurons were treated with PA or ICNs and immunolabeled them after 48 hours of treatment (FIG. 4A).

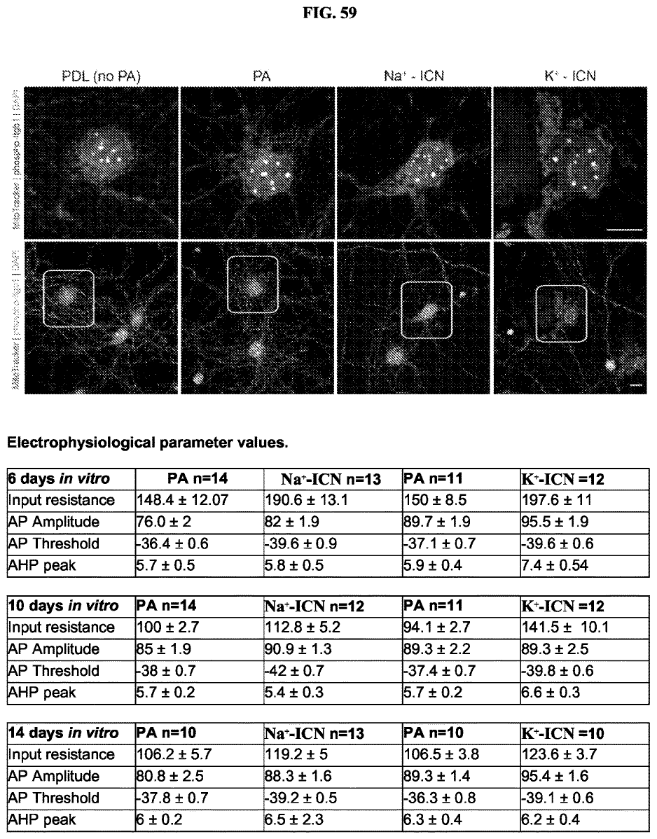

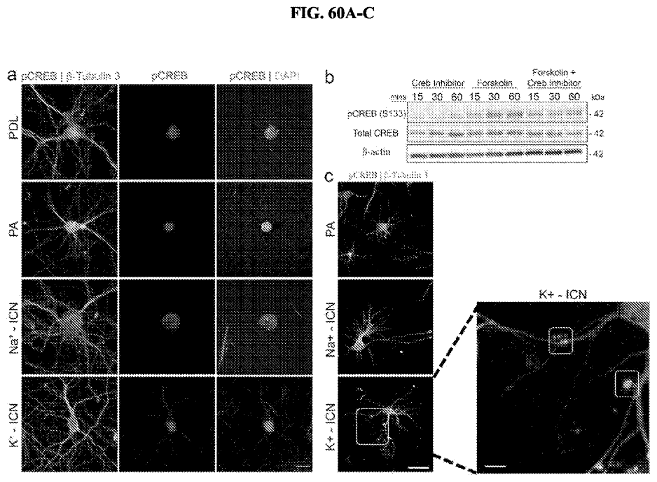

Neurons treated with either sodium or potassium ICNs showed a significant increase in the number of p37-LRP⁺ dendritic growth cones (FIG. 4B), consistent with the larger growth cones observed in FIG. 1B. Next, the levels of phosphorylated CREB were investigated; a transcription factor known to promote neuronal growth, maturation, and survival. Relative to PA controls, both ICNs led to an increased number of pCREB⁺ nuclei (FIG. 4C), resulting in longer neurites (FIG. 4D). These findings were consistent with previous studies indicating that KCl depolarization results in increased pCREB protein levels. Since cesium inhibits potassium channels, a Cs⁺-ICN was also prepared and was observed to have poor cell survival in vitro (FIG. 59). To evaluate if the upregulation of pCREB was persistent beyond 48 hours, immunolabeling and immunoblotting were conducted at later time points. Immunolabeling at 7 DIV revealed robust pCREB upregulation within nuclei in cells in contact with Na⁺-ICN and K⁺-ICN relative to control nanostructures (FIG. 60). Furthermore, immunoblots at 15 DIV demonstrated a sustained pCREB upregulation in cortical neurons treated with ICNs (FIG. 4E). Most interestingly, both Na⁺-ICN and K⁺-ICN neuronal cultures show robust pCREB levels within nuclear compartments, whereas only cortical neurons treated with K⁺-ICN exhibited increased pCREB protein levels within dendrites (FIG. 60). Previous studies have shown that increased neuronal activity results in CREB translation within axons leading to enhanced survival.

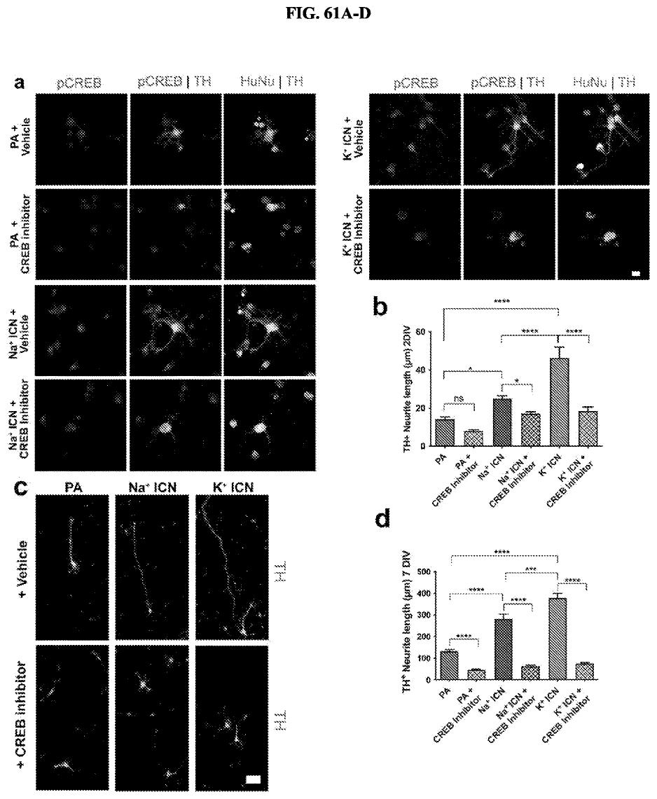

Motivated by the cell morphology observations described above, high-throughput RNA-sequencing (RNA-Seq) experiments were performed to identify potential molecular correlates that contribute to the enhancement of early neuronal development. A two-way comparative analysis was performed between samples sequenced from mouse embryonic cortical neurons treated with ICNs for 72 hours (FIG. 4F-G). Cortical neurons treated with Na⁺-ICN showed significant upregulation of 431 genes and downregulation of 163 genes, whereas those treated with K⁺-ICN showed significant upregulation of 723 genes and downregulation of 209 genes (P<0.01, n=3 libraries per condition). Focusing on the significantly upregulated transcripts for both ICNs, gene ontology (GO) analyses suggested different mechanisms for pCREB upregulation (FIG. 4F-G and FIG. 61). Specifically, key mitochondrial genes involved in oxidative phosphorylation, such as mt-Atp6, mt-Co1, mt-Co2, mt-Co3, and mt-Nd1 were significantly upregulated by both Na⁺-ICN and K⁺-ICN (FIG. 4H). However, it was observed that genes implicated in secretory vesicle formation and secretion (ChgB, VGF, Scg2 and Pam) were solely upregulated by K⁺-ICN (FIG. 4I). Moreover, the growth factor BDNF, the NMDA-receptor subunits Grin1 and Grm2, and the potassium channels Kcna1 and Kcna6 were solely upregulated by K⁺-ICN (FIG. 4I). Based on gene expression, it was concluded that distinct downstream signaling mechanisms for neuronal maturation are activated by Na⁺ or K⁺ clouds.

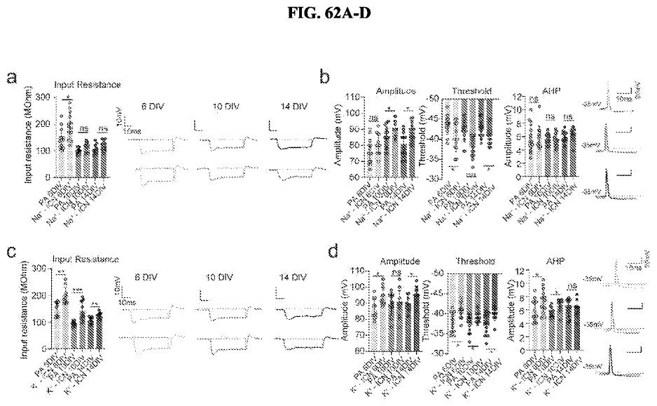

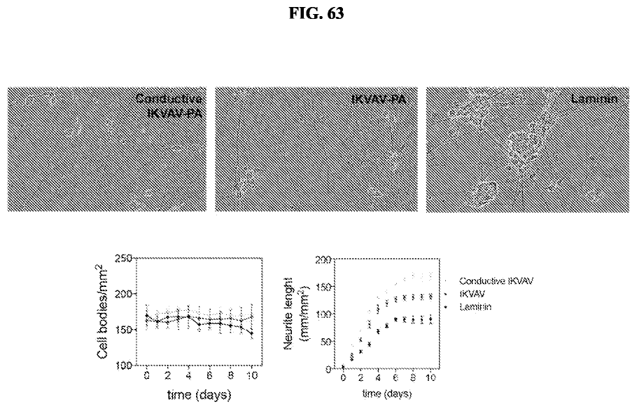

It was further assessed that the expression levels of the sodium channel Naᵥ1.6, encoded by the gene SCN8A, one of the major voltage-gated channels in the human brain and responsible for the initiation of neuronal membrane depolarization. Relative to PA controls, Na⁺-ICN and K⁺-ICN KCl cultures exhibited an upregulation of the ion channel by ~2.1-fold and ~2.5-fold, respectively (FIG. 4J-K). Using induced pluripotent stem cells (iPSCs) directly differentiated into glutamatergic neurons, it was then investigated whether these observations are conserved in human neurons. In agreement with our mouse neuronal assays, it was observed robust upregulation of pCREB along dendritic compartments in these neurons when treated with K⁺-ICN, and to a lesser extent in those treated with Na⁺-ICN (FIG. 4L-M). It was further speculated that enhanced levels of extracellular potassium generated by the presence of the cloud are more biologically potent given the low concentration of this ion in physiological fluids relative to sodium. Indeed, it is well known that an increase in extracellular potassium concentration causes global membrane depolarization leading to calcium influx and ultimately pCREB-dependent maturation. Following, CREB-dependent developmental program activation using iPSCs were probed directly and differentiated dopaminergic neurons. It was found that dopaminergic neurons enhanced growth cone area and neurite length with both ICNs, which could be reversed with a CREB-specific antagonist (FIG. 62-63). Based on these experiments, it was concluded that the sodium and potassium clouds activate distinct gene expression programs that culminate in the upregulation of pCREB, thus leading to enhanced neuronal maturation.

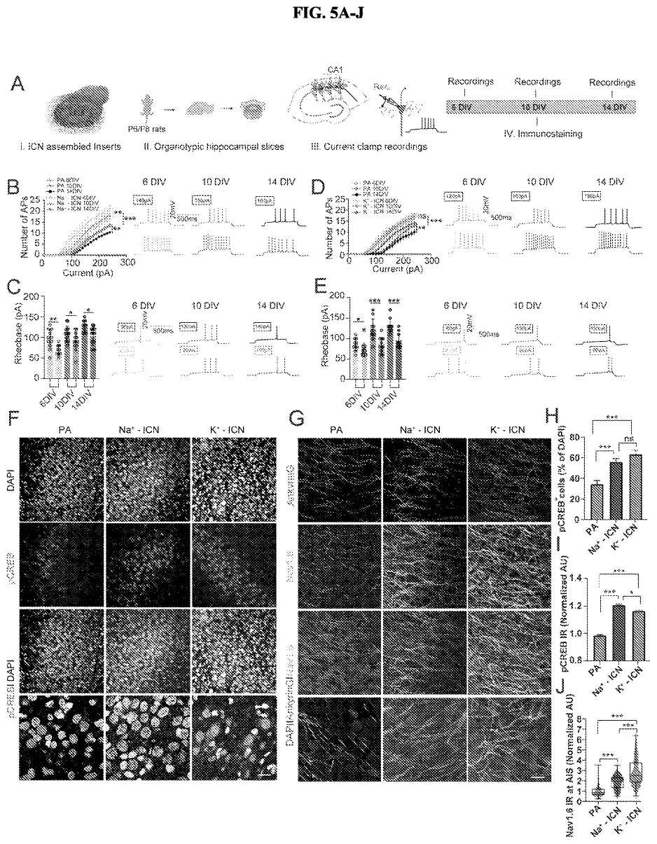

To physiologically dissect the unique responses activated by Na⁺-ICN vs. K⁺-ICN, we explored the effect of ICNs on the stability of the hippocampal network. This well-established ex vivo model allows for the ability to reliably acquire electrophysiological data using organotypic hippocampal slice cultures from neonatal rats. A PA or ICN layer was prepared on top of Millicell® cell culture inserts, inserted hippocampal organotypic slices (including the entorhinal cortex), and measured multiple electrophysiological parameters from CA1 pyramidal neurons at 6, 10, and 14 DIV using whole-cell current clamp recordings (FIG. 5A). Given the known heterogeneity of the hippocampus, the CA1 pyramidal neuron excitability was measured in the dorsal area. Importantly, the dorsal hippocampus is most active during spatial memory so its key role in synaptic and intrinsic plasticity phenomena is well established.

Next, the intrinsic excitability of CA1 pyramidal neurons with progressive somatic current injections was assessed (FIG. 5B, D, Table 1). Na⁺-ICN had a potent effect on action potential number at 6 DIV and a significant effect over time compared to the PA control. Rheobase, a measure of the minimal current injection required to induce membrane depolarization, significantly decreased at 6, 10 and less dramatically at 14 DIV, indicating a maximal effect at an earlier stage in neurons exposed to Na⁺-ICN compared to the PA control (FIG. 5C, Table 1). Similar experiments with K⁺-ICN layers revealed a slight increase in the steady-state action potential number observed at 6 DIV, which persisted and became highly significant at 10 and 14 DIV (FIG. 5D, Table 1). The rheobase was consistently lower at all time points when utilizing K⁺-ICN layers, indicating a persistent effect on excitability (FIG. 5E, Table 1). Next, a series of intrinsic parameters were measured to analyze in detail the potential membrane excitability mechanisms induced by both ICNs relative to the PA control. The input resistance, a good indicator of membrane passive properties, increased only at 6 DIV in CA1 pyramidal neurons cultured with Na⁺-ICN relative to the PA control (Table 1). Conversely, the input resistance of neurons cultured on K⁺-ICN was significantly larger than that in the PA control over time (Table 1). These results reveal a larger input resistance at the three time points analyzed for K⁺-ICN layers, but only at 6 DIV for the Na⁺-ICN substrates. Other parameters that indicate how membrane properties change in response to intrinsic plasticity are the action potential amplitude, threshold, and after-hyperpolarization potential (AHP). Following, these parameters were measured and each of the neurons analyzed. CA1 pyramidal neurons cultured on Na⁺-ICN showed a significant increase in amplitude and more negative potential threshold values at 6, 10, and 14 DIV, but no change in AHP (Table 1). Interestingly, K⁺-ICN layers revealed a significantly increased amplitude at 6 and 14 DIV, while threshold potential was found to be more negative at all time points. Additionally, AHP amplitude was significantly increased for K⁺-ICN at 6 and 10 DIV, but not at 14 DIV (FIG. 63 and Table 1). These results suggest that the effects of ICNs on intrinsic excitability are exerted by different mechanisms and K⁺-ICN shows a more persistent physiological effect. Sodium is smaller and more hydrated than potassium and therefore the liberation of these two essential cations from their respective clouds to promote neuronal excitation and repolarization may not be the same. For example, the exchange rate of sodium and potassium within the clouds, and thus their availability to cellular channels or pumps may not be symmetrical.

TABLE 1
Membrane excitability and rheobase values.

| 6 days in vitro | PA n = 14 | Na⁺-ICN n = 13 | PA n = 11 | K⁺-ICN = 12 |
|---|---|---|---|---|
| Spikes evoked at | 16.4 ± 1.1 | 20.9 ± 0.9 | 16.2 ± 0.9 | 17.9 ± 0.9 |
| 240 pA | | | | |
| Rheobase | 95.7 ± 5.6 | 67.6 ± 3.9 | 86 ± 4.7 | 72.6 ± 4.9 |
| 10 days in vitro | PA n = 14 | Na⁺-ICN n = 12 | PA n = 11 | K⁺-ICN = 12 |
| Spikes evoked at | 13.2 ± 0.7 | 18 ± 1.1 | 12.8 ± 0.9 | 17.8 ± 0.7 |
| 240 pA | | | | |
| Rheobase | 109.3 ± 4.8 | 90.8 ± 6.4 | 121.8 ± 7.7 | 83.3 ± 5.5 |
| 14 days in vitro | PA n = 10 | Na⁺-ICN n = 13 | PA n = 10 | K⁺-ICN = 10 |
| Spikes evoked at | 11.1 ± 0.5 | 13.7 ± 0.4 | 10.3 ± 0.9 | 15 ± 0.8 |
| 240 pA | | | | |
| Rheobase | 127 ± 4.4 | 103.1 ± 6.2 | 131 ± 5.4 | 93.6 ± 5.4 |

To understand further the neurodevelopmental and electrophysiological findings, organotypic slices with pCREB were immunolabeled and were treated with PAs or ICNs for 10 days. These experiments were conducted based on previous observations on the upregulation of pCREB in neurons treated with ICNs. In the organotypic slices exposed to both ICNs, it was observed that a robust increase in the number of pCREB⁺ cells relative to PA controls (FIG. 5F, H, I). Additionally, pCREB expression levels were also significantly upregulated with both ICNs relative to the PA control (FIG. 51). This increase in pCREB activation is accompanied by a dramatic increase in the expression levels of the sodium channel Naᵥ1.6 (FIG. 5G, J), as previously observed in mouse cortical neurons (FIG. 4K-L). Neurons on Na⁺-ICN substrates exhibited a ~1.8-fold upregulation of Naᵥ1.6 protein levels at the AIS relative to PA controls, whereas those on K⁺-ICN substrates showed an ~3-fold increase (FIG. 5G, J). Voltage-gated sodium channels (VGSCs) are known to be critical in the generation and propagation of action potentials in neurons. Thus, these results demonstrate that the increase in excitability observed in our electrophysiological experiments is associated with strong upregulation of pCREB, which directly increases Naᵥ1.6 protein levels.

Following the previous experiment, the next strategy was to create large supramolecular nanostructures with completely ionized molecules, which would normally prevent their self-assembly due to electrostatic repulsion. This was accomplished using amphiphilic organic cations with delocalized charge that co-assemble with peptides. The supercharging of the nanostructures resulted in the formation of an unusually dense cloud of counterions on the exterior of the assemblies. It was then discovered that ion cloud nanostructures prepared in saline exhibited a strong effect on neuronal development as indicated by the appearance of larger growth cones, increased neurite length, greater synaptic marker expression, and a more mature electrophysiological profile. By preparing nanostructures with clouds that initially contained exclusively sodium or potassium, the biological mechanisms were demonstrated to be driven by CREB-dependent gene expression programs in mouse and human neurons. Importantly, using organotypic hippocampal slices we observed signatures of ion cloud-driven bioactivity. Specifically, ex vivo rat model experiments revealed enhanced levels of membrane excitability, increased pCREB activation, and greater sodium channel expression. The molecules forming the assemblies are biodegradable and it was anticipated that a finite lifetime of the ion cloud as it equilibrated within its environment. Taken together, these data suggest that these supramolecular systems could be used to transiently activate neuronal function in diseased states or aging, provide neuroprotection, or promote neural regeneration.

### Example 2

### NMR Spectroscopic Analyses

AcVVAAEE peptide was used in analysis conducted during developments of embodiments herein to observe peptide NMR signals in the case of non-assembling system with the same amino acid sequence as studied PA in aqueous environment using solely solution NMR. This compound was selected because it is highly challenging to obtain spectral features of the PA monomer in aqueous solution due to low critical aggregation concentration of this PA. Additionally, it has been challenging to observe the spectral features of PAs in PA and OC-PA assemblies under conditions of interest utilizing solution NMR approach. Therefore, supramolecular nanostructures were pelleted using ultracentrifugation and HRMAS NMR measurements were conducted with ¹H detection to rationalize the huge difference in the amount of monovalent ions condensed on the surface of PA and OC-PA nanostructures. Binding of Na⁺ and K⁺ was studied using ²³Na NMR in solution.

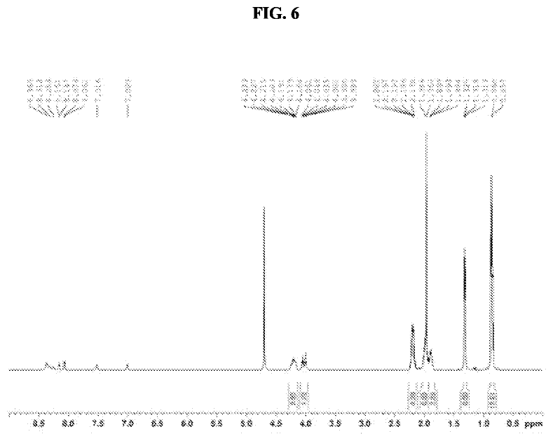

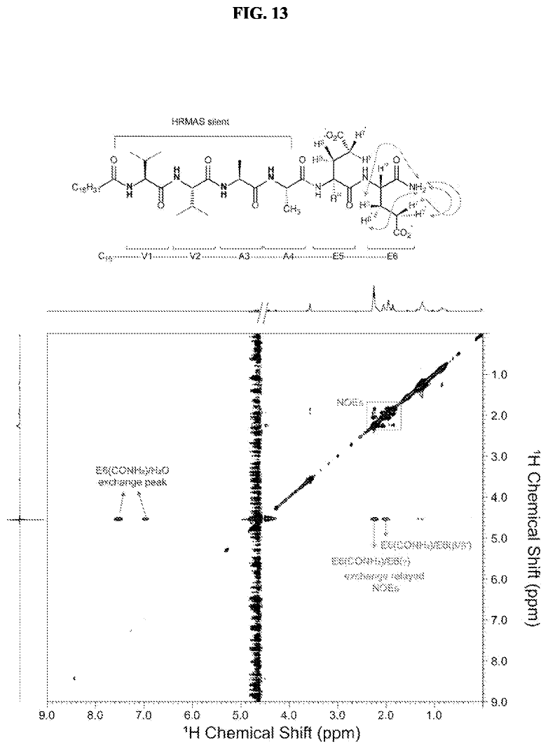

NMR Sample preparation for AcVVAAEE peptide. AcVVAAEE (6.6 mg, 0.01 mmol) was dissolved in 1 ml of 1×PBS. Using 1 M NaOH the pH was adjusted to pH=7.4 (pH-meter), and to that solution 100 μL of D₂O was added; 600 μL of the sample was transferred into 5 mm Wilmad NMR tube. Spectra were recorded using Bruker Neo 600 MHz system with QCI-F cryoprobe at 298 K. For 2D homonuclear experiments, typically a spectral width of 2048×512 complex points were used in direct (F2) and indirect (F1) dimensions and pulse sequences with excitation sculpting for water suppression together with added elements for zero-quantum transition artifacts suppression.
AcVVAAEE Resonance Assignments. The analysis started by identifying the methyl group from acetamide, which gives a characteristic singlet at δ=1.97 ppm in ¹H NMR (FIG. 6). The corresponding methyl carbon resonance was then found using ¹H—¹³C HSQC while C═O (Ac) was found using ¹H—¹³C HMBC. When CO(Ac) was found, α-¹H/¹³CO^{amide} walk in ¹H—¹³C HMBC to assign ¹H and ¹³C resonances from the peptide backbone was performed. The corresponding side chain protons were assigned using ¹H—¹H 2D TOCSY NMR experiments. Then the α- and side chain ¹³C resonances were assigned using ¹H—¹³C HSQC. Additionally, sharp resonances and unsignificant chemical shift dispersion of α- and side chain protons indicates random coil conformation of this peptide in solution. The side chain ¹H and ¹³C resonances coming from A3 and A4, as well as E5 and E6 are strongly overlapping. This is in line with random coil conformation. The α-CH and CO^{amide} region in our ¹H—¹³C HMBC were highly resolved, so resonances coming from α-CH and CO^{amide} from peptide backbone were possible to assign fully and unambiguously.
HRMAS NMR Sample Preparation and Spectra Acquisition. The PA filaments, PA micelles, and OC-PA filaments, were prepared following the protocols given in methods and materials. Obtained materials were then subjected to ultracentrifugation for 4 h at 15° C. and ~600 000 g using a Sorvall MTX preparative ultracentrifuge and S140-AT fixed angle rotor. In all studied cases the centrifugation led to formation of the pellets and supernatants except for PA micelles. In the case of PA micelles, 1 mL of 2 mM PA sample were spun and made in the presence of 6 equiv. of NaOH and collected a 100 μL of highly viscous solution at the bottom of the ultracentrifugation tube. The corresponding samples (pellets or highly viscous solutions isolated from the bottom of the ultracentrifugation tube) were spiked with 10% D₂O, vortexed and then transferred to a Bruker Kel-F insert using tabletop centrifuge (4000 rpm for 10 min) and sealed using a plug and sealing screw to prevent sample dehydration during experiments. The insert was placed into a Bruker 4 mm zirconia MAS rotor and capped with a Kel-F drive cap. NMR spectra were recorded using an 800 MHz Bruker Avance III HD spectrometer equipped with a 4 mm high-resolution magic-angle spinning (HR-MAS) probe, and processed and analyzed using Topspin 4.1.4 and Dynamic Center from Bruker. The MAS rate and sample temperature during the experiments were actively controlled at 10 kHz and 46° C., respectively. Temperature calibration on HRMAS probe was done using 99.8% MeOD in placed in KelF insert. Temperature calibration curves were generated for 298-323 K at 5 kHz and 10 kHz spinning speeds using a calctemp macro in TopSpin for determining those temperatures.
Resonance Assignments for PA filaments. The first observation made on the measured ¹H HRMAS NMR on pelleted PA filaments was that it presents a much smaller number of ¹H signals than what is expected based on the chemical structure of the PA. Relying on chemical shift and splitting patterns found in the corresponding AcVVAAEE control, as well as internuclear contacts measured via ¹H—¹H TOCSY, ¹H—¹H NOESY and ¹H—¹³C HSQC. It was discovered that only glutamic acid residues are mobile enough to be captured using HRMAS technique. This is in line with prior reports which found using EPR spectroscopy on labeled PA molecules, with the same chemical structure as PA used herein, that core of the filaments is highly rigid, which is in positive correlation with the absence of majority of signals in ¹H HRMAS corresponding to palmitic acid as well as VVAA peptide segment. ¹H—¹H NOESY spectrum (FIG. 13) revealed exchange peaks among carboxamide (CONH₂) hydrogens and water on the millisecond timescale. Importantly, the chemical shift of the PA CONH₂ hydrogens (δ=7.52 and 7.012 ppm) aligns well with CONH₂ ¹H shifts measured on AcVVAAEE peptide via solution NMR (δ=7.55 and 7.00 ppm). This comparison was used to unambiguously assign these resonances. After this, it was possible to assign the cross correlations emerging due to magnetization transfer among water and sidechain E-hydrogens as an exchanged relayed NOEs among water and E6 sidechain hydrogens (FIG. 13). Using ¹H—¹H TOCSY, it was possible to map out a whole spin network for both E5 and E6. Lastly, ¹H—¹³C HSQC (FIG. 50) corroborated these assignments and provided the corresponding ¹³C chemical shifts.
Solution HR NMR studies for OC binding to PA. Since biological experiments revealed the highest cell viability with assemblies containing ethyl methyl imidazolium cation (OC), the next study focused on NMR analysis specifically on the corresponding OCPA samples. Here, ¹H NMR spectra was measured to find that the NMR signals corresponding to OC (FIG. 51) are significantly exchange broadened for the corresponding OCPA sample relative to OC by itself. By utilizing ¹H DOSY NMR (FIG. 51D-E) in solution it was found that lower diffusion coefficient (D) for OC in OC-PA sample relative to sample containing OC alone at the same concentration, was additional evidence of binding of the organic cation to PA nanostructures. In such samples, the OC is partitioned among free (OC_{f}, fast diffusing) and bound (OC_{b}, slow diffusing) and the measurement captures the population weighted average diffusion constant (Dₐₚₚ=p_{f}D_{f}+p_{b}D_{b}; here p_{f} and D_{f} refer to population and diffusion coefficient of OC_{f}, respectively, while p_{b} and D_{b} refer to population of and diffusion coefficient of OC_{b}, respectively).
Resonance Assignments for PA micelles. ¹H HRMAS NMR on PA micelles presents the expected number of ¹H signals based on PA chemical structure. Relying on chemical shift and splitting patterns found in the corresponding AcVVAAEE control led to the discovery of a full peptide backbone which was mobile enough to be captured using HRMAS technique and was in sharp contrast to PA filaments where glutamic acid residues appear only. Importantly, carboxamide (CONH₂) and water hydrogens exchange rapidly on the NMR timescale in this highly basic sample, as expected, so the chemical shift of those hydrogens are not available. Aliphatic peaks were assigned based on the expected chemicals shifts (i.e., as found in the AcVVAAEE control) and internuclear correlations found in ¹H—¹H TOCSY and ¹H—¹³C HSQC.

TABLE 2
¹H and ¹³C chemical shifts (ppm) for AcVVAAEE and C16VVAAEE
PA micelles, PA filaments and OC-PA filaments measured
via solution (HR) and semi-solid (HRMAS) NMR.

| AcVVAAEE | NH | Ha/Ca | Hb/Hb′/Cb | Hγ/Cγ | Cδ |
|---|---|---|---|---|---|
| V1 | 8.07 | 4.00/62.50 | 1.94/—/32.65 | 0.87/20.49 | — |
| V2 | 8.15 | 4.05/61.98 | 1.97/—/32.84 | 0.85/21.07 | — |
| A3 | 8.26 | 4.23/52.33 | 1.32/—/19.22 | — | — |
| A4 | 8.31 | 4.21/52.50 | 1.32/—/19.22 | — | — |
| E5 | 8.37 | 4.16/56.50 | 1.88/1.99/30.44 | 2.19/36.25* | 184.00* |
| E6 | 8.37 | 4.19/56.61 | 1.88/1.99/30.23 | 2.19/36.35* | 184.06* |

| | |
|---|---|
| CO₂NH₂ | 7.52, 7.00 |
| Ac | CH₃ 1.96/24.34; CO 177.03 |

Solution NMR.
*It was not possible to unambiguously assign to E5 or E6.

| PA micelles | NH | Ha/Ca | Hb/Hb′/Cb | Hγ/Cγ | Cδ |
|---|---|---|---|---|---|
| V1 | — | 4.26/— | 2.05/— | 0.91/20.4 | |
| V2 | — | 4.26/— | 2.05/— | 0.90/21.1 | |
| A3 | — | 4.35/— | 1.37/19.4 | — | |
| A4 | — | 4.41/— | 1.37/19.24 | — | |
| E5 | — | 4.26/— | 2.05/1.96/30.3 | 2.24/36.12 | |
| E6 | — | 4.26/— | 2.05/1.96/30.3 | 2.24/36.12 | |

| | |
|---|---|
| CO₂NH₂ | — |
| C16 | CH₃(16): 0.85/16.2; CH₂(2): 2.24/38.1; CH₂(4): |
| | 1.58/28.26; CH₂(3,5-15): 1.25/24.74, 31.7, 34.1 |

¹³C shifts were read from 1H-13C HSQC.

| PA filaments | NH | Ha/Ca | Hb/Hb′/Cb | Hγ/Cγ | Cδ |
|---|---|---|---|---|---|
| V1 | — | — | — | 0.90 | — |
| V2 | — | — | — | 0.90 | — |
| A3 | — | — | — | — | — |
| A4 | — | — | — | — | — |
| E5 | — | 3.57/56.6 | 1.95/1.84/32.9 | 2.26/36.1 | — |
| E6 | — | 4.27/56.2 | 2.05/1.95/30.4 | 2.19/36.2 | — |

| | |
|---|---|
| CO₂NH₂ | 7.55, 7.00 |
| C16 | CH₃ 0.83/—; CH₂ 1.25/32.22 |

¹³C shifts were read from 1H-13C HSQC.

| OCPA | | | | | |
|---|---|---|---|---|---|
| filaments | NH | Ha/Ca | Hb/Hb′/Cb | Hγ/Cγ | Cδ |
| V1 | — | — | — | — | — |
| V2 | — | — | — | — | — |
| A3 | — | — | — | — | — |
| A4 | — | — | — | — | — |
| E5 | — | — | 2.03/1.95— | 2.25/— | — |
| E6 | — | — | 2.05/1.95/30.4 | 2.25/— | — |

| | |
|---|---|
| CO₂NH₂ | — |
| C16 | CH₃ 0.891/—; CH₂ 1.301/— |

Binding of organic cations (OCs) to PA. Initial screening of interactions of five different OCs with PA was performed using ¹H NMR spectroscopy in solution (FIG. 53). Here, ¹H signals lineshape from the ¹H NMR spectra of OCs were compared and analyzed against the corresponding ¹H signals from OC-PA samples. It is expected that ¹H signals will experience broadening if binding is occurring. In all cases we observed significant broadening except for tetramethylammonium (OC4), indicating its poor binding to PA nanostructures.
¹H DOSY NMR measurements. Spectra were measured by monitoring the change in intensity of the corresponding resonances as a function of the field gradient strength g (G/cm). A 2D sequence was used for diffusion measurement based on stimulated echo and LED which applies bipolar gradient pulses for diffusion (2 spoil gradients) together with water presaturation during relaxation delay (ledbpgppr2s Bruker sequence). ¹H DOSY data were first processed in Topspin (Fourier transformation, phasing, and baseline correction) and then the peak intensities of the corresponding resonances were extracted and fitted to the Stejskal-Tanner equation (Eq. 1) to give the value of diffusion coefficient D (m²/s). Solution ¹H DOSY NMR data were fit to Eq. 1 using NMRTools.jl{, #28110}. ¹H HRMAS DOSY NMR data were fit using the Eq. 1 using Dynamics Center 2.6.3 software.

$$\begin{matrix}Equation 1 & \\ I = {I}_{0} exp [ - {γ}^{2} {g}^{2} {δ}^{2} {σ}^{2} ( Δ - δ / 3 ) D ] . & ( Eq . 1 )\end{matrix}$$

In the Eq. 1, I and I₀ are the signal intensities in the presence and absence of gradient pulses, respectively; γ is the gyromagnetic ratio; g and δ are the gradient strength and their duration, respectively; σ=0.9, is the shape factor for the SMSQ gradient used in the experiment; Δ is the time separation between the edges of the gradient pulses.

At this point it was recognized that binding of OC can be due to electrostatic association on the surface of the nanoribbons and/or inclusion of OCs deeper in the core of PA nanostructure. Importantly, the diffusion properties of core-bound (OC_{c}) and surface-bound (OCₛ) organic cation as well as their exchange rate with OC_{f} can be expected to differ and that via solution NMR it was hypothesized that the contributions of OC_{f} and OCₛ rapidly exchanging were primarily be captured on the ¹H NMR timescale (FIG. 51).

Solution (HR) and semi-solid (HR-MAS) NMR studies for OC binding to PA. To gain more insights into binding of OC to PA we assembled the two at different OC/PA ratios (OC/PA=2, 4, 8, and 16) while keeping the concentration of the PA constant (c(PA)=10.0 mM). These samples were ultracentrifuged and resulting supernatants were analyzed using solution HR NMR in the presence of DSS (sodium trimethylsilylpropanesulfonate) as internal standard (FIG. 52). By integrating CH₃(Et) signal from OC against CH₃ signal from DSS the concentrations of OC in the supernatant were calculated. As seen in Table 3, the concentration of the OC in the supernatant ([OC]ₛₙ) is significantly lower relative to initial concentration of OC ([OC]₀) which goes in line with binding of OC to PA filaments.

TABLE 3
Concentration of OC remaining in the supernatant
upon pelleting of OCPA samples.

| [PA]₀, mM | [OC]₀, mM | [OC]ₛₙ, mM |
|---|---|---|
| 10.00 | 20.00 | 4.66 |
| 10.00 | 40.00 | 14.96 |
| 10.00 | 80.00 | 31.64 |
| 10.00 | 160.00 | 58.45 |

[PA]₀—concentration of PA before ultracentrifugation;
[OC]₀—concentration of OC before ultracentrifugation
[OC]ₛₙ refers to concentration of OC in the supernatant.

Four gel pellets obtained in these experiments were analyzed using high resolution magic angle spinning (HRMAS) NMR spectroscopy. FIG. 56 shows a region of the four ¹H HRMAS NMR spectra featuring aromatic hydrogens from OC. FIG. 56A displays an overlay of four ¹H HRMAS NMR spectra acquired under identical conditions, without any intensity adjustments. This revealed a consistent increase in intensity of aromatic resonances with an increment in the molar equivalent of OC added. Importantly, the emergence of the second set of aromatic resonances was observed. These broad peaks become more prominent at higher loadings of OC (FIG. 56B).

¹H HRMAS Resonance Assignments for OC-PA assembly. The ¹H HRMAS NMR measured on pelleted OC-PA filaments, made with 16 equiv. of OC relative to PA, presents two sets of ¹H OC signals, broad and sharp. By comparing the chemical shift and splitting patterns found in ¹H NMR of free OC with signals measured via ¹H HRMAS NMR on OC-PA as well as internuclear contacts measured via ¹H—¹H HRMAS TOCSY and NOESY, each peak was able to be assigned to the corresponding hydrogen from OC, as shown in FIG. 57. Importantly, only a few PA resonances were detected, which were much weaker relative to OC component, as expected. These findings correspond to glutamic acids side chain hydrogens and mobile hydrogens from the palmitic acid tail. Next, two sets of OC resonances were assigned, sharp and board based on ¹H HRMAS T₂ and DOSY. Sharp signals featured with longer T₂, and large D coefficient were assigned to OC_{f} (OC free in liquid phase) and OCₛ (electrostatically attracted OC on the surface of filament) which are in fast exchange relative to ¹H chemical shift timescale. Broad ¹H signals featured with short T₂ times and lower D coefficient were assigned to OC_{c} (OC bound within the core of the PA filament).
²³Na NMR Spectroscopy in Solution. NMR measurements on ²³Na are extensively employed to study the structure and dynamics of aqueous heterogeneous systems, including biopolymer gels and even biological tissues. ²³Na, with a nuclear spin of 3/2 and nearly 100% natural abundance, generates the second strongest NMR signal in biological tissues, following protons. The primary relaxation mechanism involves the fluctuating quadrupolar interaction between the ²³Na nuclear electric quadrupole moment and electrostatic field gradient at the location of the nucleus under observation. As a result, NMR relaxation time measurements can provide insights into the local distribution of electric charges and the corresponding charge movements.

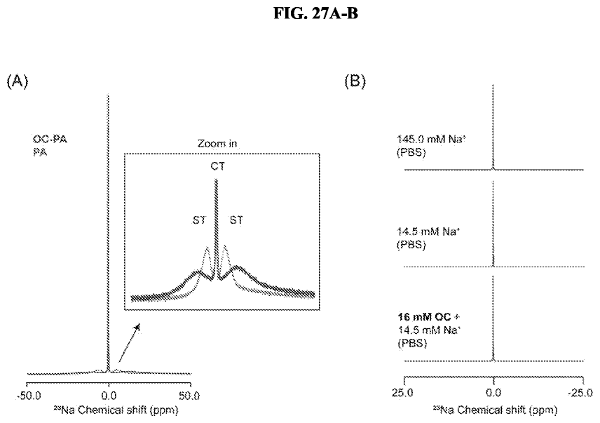

First, acquired was the ²³Na NMR in solution for 10 mM PA and OCPA samples. It was found that there were spectral differences in the positions and linewidths of satellite transitions (ST; FIG. 27). According to the measurements conducted on the corresponding controls containing buffer only for PA or buffer and OC for OCPA, there was no signs of ST peaks, which was expected for ²³Na⁺ in isotropic solutions (extreme narrowing limit). Therefore, these originate solely from the sodium bound to supramolecular nanostructures. The appearance of STs is determined by dynamics of Na⁺ binding (Na⁺+PA [symbol] PA-Na⁺; Na⁺+OCPA [symbol] OCPA-Na⁺) to supramolecular nanostructures and quadrupolar coupling constant (QCC) associated with the corresponding bound state of sodium (i.e., PA-Na⁺ and OC-PA-Na⁺). The measurements of the corresponding QCCs were not measured directly therefore, longitudinal (R₁) and transverse (R₂) ²³Na relaxometry measurements using PA and OCPA samples containing 1 mM PA at 14.5 mM total sodium concentration were used. under a 10-fold diluted conditions, in order to be closer to extreme narrowing regime and enable relaxation data analysis.

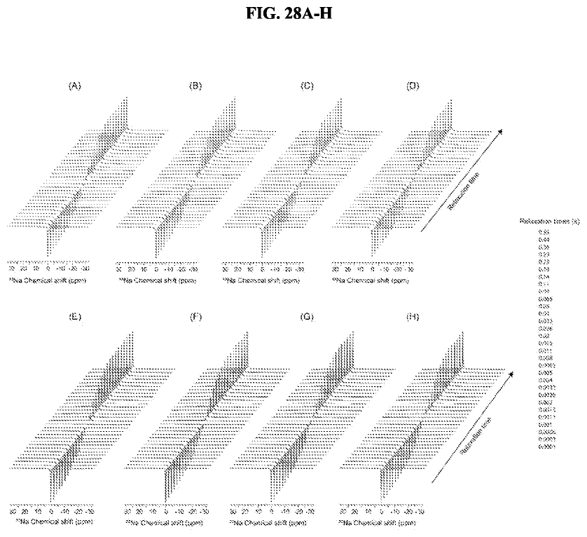

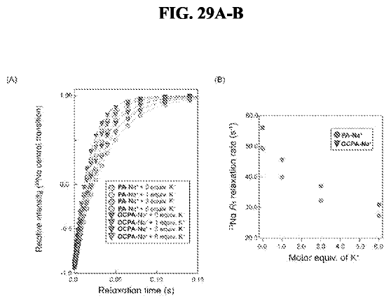

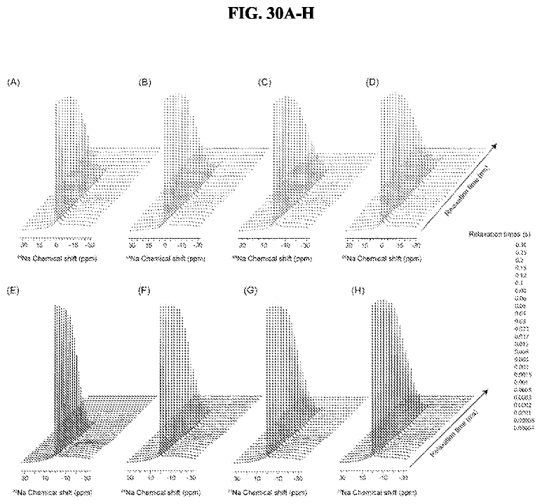

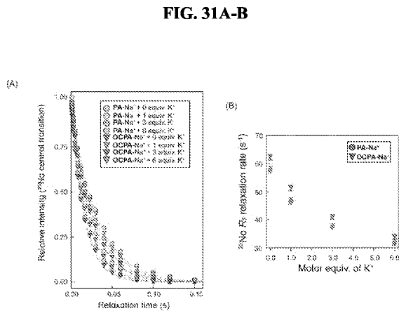

Considering the equilibria Na⁺+PA [symbol] PA-Na⁺ and Na⁺+OCPA [symbol] OCPA-Na⁺ it can be expected that the measured R₁ and R₂ values will be a population weighted average of R_{1freeNa+} and R_{1bound Na+} and R_{2freeNa+} and R_{2bound Na+}, respectively (i.e., R_{1,obs}=p_{free}R_{1freeNa+}+p_{free}R_{1boundNa+} and R_{2,obs}=p_{free}R_{2freeNa+}+p_{free}R_{2boundNa+}). The R₁ and R₂ values determined are shown in Table 4, while the corresponding measurements and fittings are given in FIGS. 28-31. As expected, measured R₁ and R₂ values are very similar in the corresponding controls, which is expected for extreme narrowing limit. R₁ and R₂ values determined for the PA and OC-PA samples are much higher than in the corresponding controls (Table 4), indicating the binding of Na⁺ to supramolecular filaments. Importantly, the R₁ and R₂ values determined for the PA are considerably smaller relative to the corresponding OC-PA sample.

To obtain the insight into K⁺ binding we utilized ²³Na NMR relaxometry in a Na⁺/K⁺ competitive binding assay (Table 4, FIGS. 28-31) where KCl was incrementally added to PA-Na⁺ and OCPA-Na⁺ samples. These relaxometry measurements revealed the gradual liberation of Na⁺ ions into solution as more KCl was added and K⁺ ions became condensed in the cloud (i.e., PA-Na⁺+K⁺ [symbol] PA-K⁺+Na⁺ and OCPA-Na⁺+K⁺ [symbol] OCPA-K⁺+Na⁺) as indicated by a reduction in ²³Na R₁ and R₂ (Table 4, FIG. 28-31).

TABLE 4
Tabulated ²³Na R₁ and R₂ determined via solution NMR.

| Molar equiv. of | | | | | | ΔR₂ (PA − |
|---|---|---|---|---|---|---|
| K⁺ | R₁(PA), s⁻¹ | R₁(OCPA), s⁻¹ | ΔR₁ | R₂(PA), s⁻¹ | R₂(OCPA), s⁻¹ | OCPA) |
| 0 | 49.2 ± 0.4 | 56.0 ± 0.4 | 6.8 | 57.8 ± 1.2 | 62.3 ± 1.1 | 4.5 |
| 1 | 39.9 ± 0.2 | 45.4 ± 0.3 | 5.5 | 46.6 ± 1.1 | 51.3 ± 1.0 | 4.7 |
| 3 | 32.2 ± 0.2 | 36.8 ± 0.2 | 4.6 | 37.5 ± 1.1 | 41.0 ± 0.8 | 3.5 |
| 6 | 27.3 ± 0.1 | 30.8 ± 0.1 | 3.5 | 31.9 ± 1.1 | 34.1 ± 0.8 | 2.2 |
| Control (PBS) | 17.8 ± 0.1 | 18.1 ± 0.1 | 0.3 | 18.4 ± 0.3 | 19.1 ± 0.2 | 0.7 |

Control for PA was PBS containing 14.5 mM Na⁺. Control for OCPA was PBS containing 14.5 mM Na⁺ and 16 mM OC.

²³Na NMR measurements in solution. Measurements are performed on 600 MHz (¹H frequency; 159 MHz ²³Na frequency) NMR spectrometer equipped with BBFO Smart Probe with Z-Gradient. For each sample, a 90° pulse calibration was done prior to acquisition of single excitation ²³Na and used in the corresponding ²³Na R₁ and R₂ relaxation measurements. ²³Na R₂ relaxation measurements were performed using a Hahn echo pulse sequence, with relaxation times from 1 ms to 300 ms. ²³Na R₁ relaxation measurements were performed using an inversion recovery pulse sequence, with relaxation times from 0.1 ms to 560 ms. For both experiments, linear baseline correction was applied to the central transition which was then integrated over a 0.7 ppm line width. Peaks integrals were fitted as a function of relaxation time to single exponential decays for R₂ measurements, and single exponential recovery for R₁ measurements. No improvement in the quality of fits was observed when multiple exponential functions were used to fit the data. Reported uncertainties are determined from the covariance matrix of the fit.

### Example 3

### Atomistic Simulations

Molecular dynamics study of the adsorption of organic cation 1-ethyl-3-methylimidazolium chloride by a rod-like nanostructure of peptide amphiphile C16A2V2E2. A rod-like nanostructure formed by the peptide amphiphile C₁₆A₂V₂E₂ was stimulated.

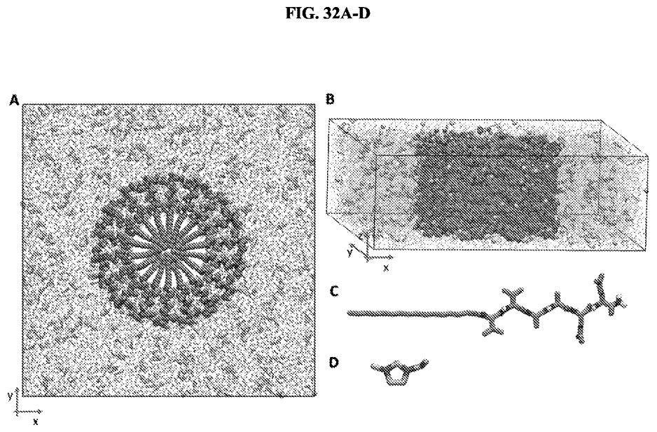

To build the nanostructure, the 18 peptide molecules was assembled in a circular cross-section on the x-y plane where each molecule is rotated around the z-direction axis by n×20°, with n=1, . . . , 18. The 18 molecules are arranged into two layers by alternating the PAs' height in the z-direction by 0.25 nm. This 18-molecule cross-section is replicated 10 times in the z-direction to generate a 5.5-nm-height nanostructure (FIG. 32A-B). The nanostructure is placed at the center of a simulation box of 16 nm×16 nm×5.5 nm and solvated using the organic cation 1-Ethyl-3-methylimidazolium chloride (OC⁺), sodium chloride, and water. The molecular structure consists of a 16-hydrocarbon linear chain attached to a peptide sequence formed by two alanine groups, two valine groups, and two glutamic acids (FIG. 32). Unless explicitly stated, each PA molecule has two negative charges (−2e) from dissociating the two glutamic acids. The systems' composition is reported in Table 5.

Next, molecular dynamics (MD) simulations were performed using the GROMOS 54a7 force field and the extended simple point charge (SPC/E) water model. The non-bonded interactions are considered using the Lennard-Jones and the Coulomb potentials for the van der Waals and electrostatic interactions, respectively. A cut-off distance of 1.2 nm was employed for the Lennard-Jones interactions. The long-range electrostatic interactions were calculated using the smooth particle mesh Ewald (PME) method, and periodic boundary conditions were imposed in the three directions. First, an energy minimization run was performed using the steepest-descent algorithm, followed by a simulation at 253.15 K for 20 ns. Then, the production run of 200 ns is performed at 298.15 K. The NPT ensemble was employed for the aneling and production run. The temperature was controlled using the Nosé-Hoover thermostat (using a characteristic time of t_{T}=2 ps), and the pressure using the semi-isotropic Parrinello-Rahman barostat algorithm. The reference pressure P_{xy}=P_{z}=1 bar, using a characteristic time of t_{P}=4 ps).

Table 5. The composition of the simulated systems is given by the number of molecules/ions in the simulation box. The last column is the OC⁺ molar concentration. In the systems of runs 1 to 6 the peptide is fully dissociated and has a charge of −2e. In the system of run 7 the peptide is partially dissociated and has a charge of −1e.

TABLE 5
The composition of the simulated systems is given by the number of molecules/ions in
the simulation box. The last column is the OC+ molar concentration. In the systems of runs 1 to 6
the peptide is fully dissociated and has a charge of −2e. In the system of run 7 the peptide is partially
dissociated and has a charge of −1e.

| Run # | C16A2V2E2 | Na⁺ | OC⁺ | CI⁻ | water | Conc[mM] |
|---|---|---|---|---|---|---|
| 1 | 90 | 70 | 280 | 170 | 30569 | 180 |
| 2 | 90 | 70 | 270 | 160 | 30569 | 160 |
| 3 | 90 | 70 | 225 | 115 | 30569 | 80 |
| 4 | 90 | 70 | 203 | 93 | 30569 | 40 |
| 5 | 90 | 70 | 191 | 81 | 30569 | 20 |
| 6 | 90 | 260 | 0 | 80 | 30569 | 0 |
| 7 | 90 | 170 | 0 | 80 | 30569 | 0 |

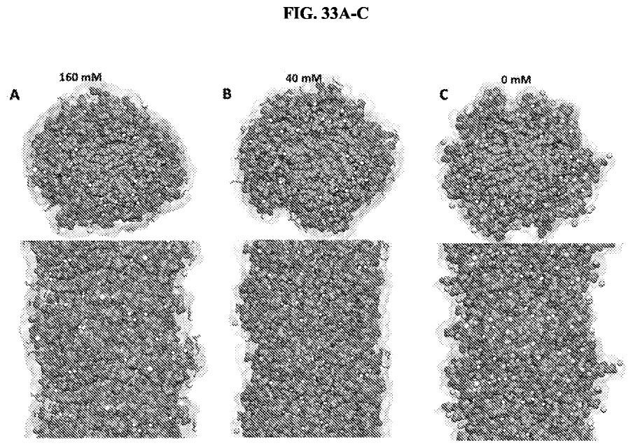

FIG. 33 shows snapshots from molecular dynamics simulations of the peptide amphiphile PA nanostructure in aqueous solutions containing 160 mM, 40 mM, and 0 mM of the organic cation 1-Ethyl-3-methylimidazolium chloride (OC⁺). Sodium chloride was approximately 150 mM. In the PA nanostructure, the amino acids form a shell around the hydrophobic core formed by the hydrocarbon chains and located at the nanostructure's center. At the beginning of the simulation, only Na⁺ ions were randomly placed inside the nanostructure, whereas the OC⁺ and Cl were in the aqueous region. At all the concentrations, it was observed that the Na⁺ ions are expelled from the nanostructure, and they are replaced by the OC⁺ ions that penetrate inside the PA nanostructure. At the OC—Cl concentrations different from zero, only small concentrations of Na⁺ ions are found inside the nanostructure in the equilibrium configurations. It was shown graphically that the OC⁺ molecules penetrate deeper inside the nanostructure at the highest concentration (FIG. 33A). In contrast, a thinner OC⁺ shell was observed at the lower concentration (FIG. 33B). In the absence of the organic cation, the nanostructure's charge was compensated by Na⁺ ions. In the absence of the organic cation, water intrudes inside the nanostructure more than in the presence of the organic cation. Therefore, OC⁺ also expelled water from the nanostructure.

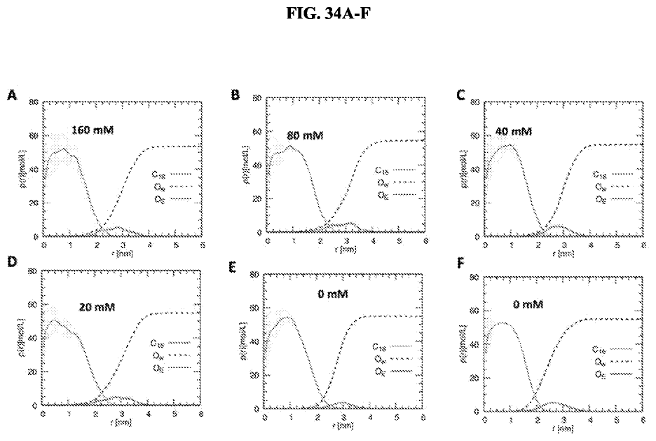

FIG. 34 shows the density profiles (as a function of the cylindrical coordinate r) of the carbon atoms from the hydrocarbon tail of the PA amphiphile, the oxygen atoms from the glutamic acid groups, and the oxygen atoms from the water molecules. To calculate the density profiles, a cylindrical coordinates system fixed at the center of mass of the rod-like nanostructure was used. The hydrophobic tails form a hydrophobic core at the nanostructure's center, whereas the glutamic acid groups were found at the outer region of the nanostructure at the aqueous interface. The water density profiles are qualitatively similar at the OC⁺ concentrations from 20 mM to 180 mM, however, without the organic cation there is a higher water intrusion into the nanostructure.

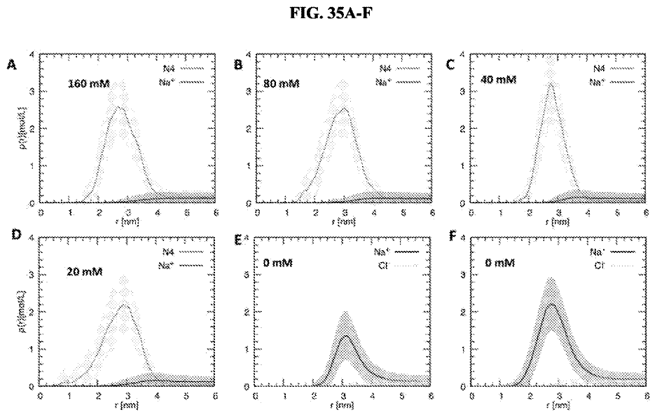

FIG. 35 shows the ionic species distribution around the rod-like nanostructure at organic cation concentrations from 160 to 0 mM. The organic cation density profiles, as a function of the cylindrical coordinate r, form a bell-shaped distribution inside the PA nanostructure. As we anticipated in the discussion of FIG. 5, the OC⁺ intrusion into the nanostructure decreases as the OC⁺ concentration decreases. At the concentrations of 160, and 80 mM, the bell-shaped distributions span from about 1.4 nm to 4.2 nm. At 40 mM the distribution spans from 1.8 nm to 4 nm. At 20 mM, the peak height decreases. In the absence of the organic cation, the nanostructure charge was compensated only by Na⁺ ions distributed from 1.6 nm to 5 nm. The absorption of the organic cation into the nanostructure changes the local environment in the nanostructure which may lead to the dissociation of the inner carboxylic groups. When the nanostructure is only partially charged, a lower peak of the Na⁺ density profile was shown implying that a much lower number of ions are necessary to screen the nanostructure's charge.

To quantify these observations, the reduced adsorption was defined as:

$$Γ = \frac{2 π {L}_{\text{?}}}{{N}_{PA}} {\int }_{\text{?}}^{\text{?}} ρ ( r ) rdr$$

$$\text{?} \text{indicates text missing or illegible when filed}$$

which is the number of particles within the cut-off radius r_{c} from the nanostructure center, ρ(r) is the particles' number density profile, L_{z} is the box length in the z-direction, and N_{PA} is the number of PA molecules. Assuming cylindrical geometry of the nanostructure, the mean electric field profile was given as:

$${E}_{r} ( r ) = \frac{1}{r {ϵ}_{0}} {\int }_{0}^{\text{?}} {ρ}_{\text{?}} ( {r}^{′} ) {r}^{′} {dr}^{′}$$

$$\text{?} \text{indicates text missing or illegible when filed}$$

ρel(r′) is the charge density profile that includes all the charged atoms in the system. The mean electrostatic potential profile was given as:

$${p}_{r} ( r ) = \frac{- 1}{r} {\int }_{0}^{\text{?}} {ρ}_{\text{?}} ( {r}^{′} ) {r}^{′} {dr}^{′}$$

$$\text{?} \text{indicates text missing or illegible when filed}$$

The water polarization was quantified as:

$${p}_{r} ( r ) = \frac{- 1}{r} {\int }_{0}^{\text{?}} {ρ}_{\text{?}} ( {r}^{′} ) {r}^{′} {dr}^{′}$$

$$\text{?} \text{indicates text missing or illegible when filed}$$

Where the negative sign accounts for the direction of the dipole moment pointing outward from the water oxygen atoms toward the water hydrogen atoms.

FIG. 35A shows the reduced ionic adsorption Γ as a function of the organic cation concentration, which accounts for the number of adsorbed ionic species per PA molecule. At the highest concentrations (180 and 160 mM), the total ionic adsorption is higher than 2, which indicates that the nanostructure adsorbs more charge than necessary to compensate for the PA molecules' charge. At the concentration of 80 mM the total ionic adsorption is approximately 2, and at the lowest concentrations (40 and 20 mM) the charge is below 2. The Na⁺ adsorption is very low in the presence of organic cation. At zero organic cation concentration, the Na⁺ ions do not overcompensate for the nanostructure charge. FIG. 35B shows the reduced water adsorption as a function of the organic cation concentration. At all the organic cation concentrations, the number of absorbed water molecules is about 45 per PA molecule. The adsorption at zero organic cation concentration increases to 58 water molecules per PA molecule, approximately.

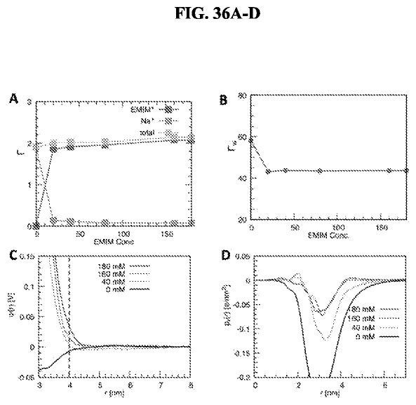

FIG. 36C shows the mean electrostatic potential at different organic cation concentrations. It was observed that the profiles drop from a high positive inside the nanostructure to zero in the aqueous phase. In the absence of organic cation, the potential is negative inside the nanostructure. The positive electrostatic potential positive values inside the nanostructure are due to the charge overcompensation due to the organic cation adsorption. The mean electrostatic potential decreases as the organic cation concentration decreases. For example, at the reference point of r=4 nm, we see a decrease from a positive value at 180 mM to a negative value at zero organic cation concentration. Despite the constant water adsorption for organic cation concentrations different from zero, the polarization enhances significantly as the organic cation concentration decreases. This effect can be attributed to a lower capability to screen the nanostructure's electric field by the Na⁺ ion than by the organic cation. Therefore, as the organic cation concentration decreases the electric field screening decreases, and in consequence, the adsorbed water is more polarized.

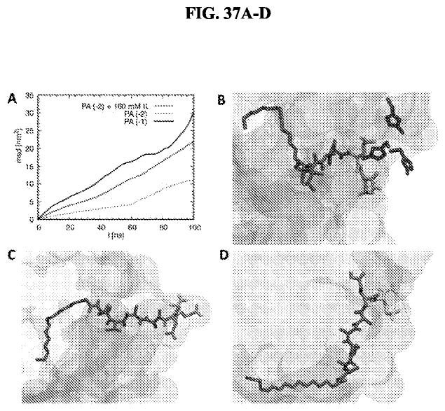

Next, the mobility of the outer glutamic acid using the mean square displacement calculated from the carboxylic acid groups was investigated. It was found that the glutamic acid mobility is higher at the organic cation concentration of 160 mM than at zero concentration. When there was no organic cation in the system, the glutamic acids charges were compensated by the Na⁺ ions. Therefore, the lower mobility could be related to the weak screening by the Na⁺ ions. Additionally, the organic cation screens more efficiently the PA charge because it penetrates the nanostructure. The location of the organic cation inside the nanostructure allows better mobility of the PA molecules. When the molecules are in an ionized state with only the outer glutamic acid dissociated the electrostatic interaction are diminished too, and this translates into a higher mobility of the glutamic acid groups. Similarly, it was found that the Na⁺ ions have a lower mobility near the fully dissociated PA molecules than in bulk due to the strong electrostatic interactions with the glutamic acid groups (FIG. 37).

The charge adsorption into a rod-like nanostructure formed by the peptide amphiphile C₁₆V₂A₂E₂ differs by changing the concentration of the organic cation OC—Cl in the aqueous phase. The organic cation was absorbed more efficiently than the Na⁺ ions into the nanostructure. The nanostructure's charge was overcompensated at organic cation concentrations of 160 mM and higher, whereas the nanostructure's charge was under-compensated at concentrations below 80 mM. The absorption of the organic cation into the nanostructure suggests a mechanism through which the inner carboxylic groups dissociate. The Na⁺ ions alone does not favor the dissociation of the inner carboxylic groups because they do not penetrate the nanostructure. However, after the inner carboxylic groups dissociate Na⁺ could replace the organic cation ions by changing the composition in the solution. In the absence of organic cation, water disrupts and intrudes into the PA nanostructure. The water absorption did not vary significantly at organic cation concentrations different from zero. However, the water polarization changes significantly decreased the organic cation concentration due to the less efficient PA's charge screening by the Na⁺ ions. Finally, the changes in the ionic environment and the ionization state change PA mobility. The weaker the electrostatic interaction the higher the PA mobility. Similarly, Na⁺ ions have a higher mobility in bulk than in the nanostructure.

### Example 4

### Molecular Theory to Predict Charge and Ion Distribution Surrounding a PA-Nanofiber

Experiments were conducted during development of the theoretical model to describe the charging of PA-fibers. The dual objective of the theoretical model was developed to gain insights into the effect that pH and other solution conditions, as well as ion type and phenomena such as ion-pairing, have off on the charge of the amino acids of PA-fiber. Additionally, the generalized Poisson-Boltzmann theoretical approach was developed to allow the prediction of the distribution of ions that are contained in the cloud surrounding the nanofiber as well as condensed on the fiber as a function of electrolyte conditions. It considers the (de) protonation of the glutamic acid residues of the PA-nanofiber as well as the possibility of ion condensation. The charge regulation of the acidic amino acids is coupled with the local dielectric environment by allowing for a varying dielectric constant that includes a position-dependent electrostatic solvation energy for all charged species.

Observed that the approach is based on a molecular approach previously developed to describe thermodynamical and structural properties of end-tethered weak polyelectrolytes, i.e., polyelectrolytes whose degree of charge is not fixed but can change depending on environmental conditions, like pH. This was particularly relevant for the predictions of the charged state of acid-ligated gold nanoparticles were in good agreement with experiments observations. Likewise, a similar approach was used to investigate the effect of solution conditions on the charge regulation of bacteriophage capsids. Finally, a similar approach was used to predict the charge and amount of absorbed charge ligand on quantum nanodots.

### Free Energy

The PA-nanofiber is in contact with an aqueous solution or reservoir of given pH that contains either monovalent NaCl, RbCl, KCl, or CsCl salt at a given concentration. The salt was assumed to be completely dissociated. For a NaCl solution, pH is adjusted by adding either HCl or NaOH. For an RbCl solution, one adds HCl of RbOH, etc. Peptide amphiphiles with the sequence C₁₆-V₂A₂E₂, which consists of an aliphatic tail of 16 hydrocarbons linked to a peptide sequence of two valines, two alanines, and two glutamic acids were considered. The carboxylic acid of the glutamic amino acid residue of this PA was found either in a deprotonated state (A⁻), a protonated state (AH), or a state in which the acid is condensed with either, Na⁺, Rb⁺, K⁺, or Cs⁺ counterion, denoted as Ana, ARb, AK, and ACs respectively. The following chemical reactions are included:

$$A H ⇄ {A}^{-} + {H}^{+} , \qquad ( S \text{.1} )$$

$${A}^{-} + {Na}^{+} ⇄ A Na , \qquad ( S \text{.2} )$$

$${A}^{-} + {Rb}^{+} ⇄ A Rb , \qquad ( S \text{.3} )$$

$${A}^{-} + {K}^{+} ⇄ A K , \qquad ( S \text{.4} )$$

$${A}^{-} + {Cs}^{+} ⇄ A Cs , \qquad ( S \text{.5} )$$

Free energy depicts the charging behavior of one PA-nanofiber in contact with an electrolyte solution described by Equation S.6 below. The first two contributions represent the mixing entropy of the solvent and mobile ions and solvent respectively. ρi(r) is the number density of species i and v_{w}, the volume of water, which is the unit of volume. The mixing entropy of the mobile ionic species also includes the standard chemical potential (μ₀ᵢ) of species i. Cylindrical coordinates were employed to reflect the symmetry of the PA nanofiber. The system is assumed to be laterally homogeneous and only explicitly anisotropic in the radial direction (r). The function, A(r)dr, is the cylindrical volume element per unit length, L. The next four contributions to the free energy describe the acid-based chemical equilibrium of the glutamic acid groups and the ion condensation of Na⁺, Rb⁺, K⁺, and Cs⁺. Here, <ρ_{Glu}(r)>, is the number density of the glutamic acid groups. The variable, f_{A-}(r), is the fraction of glutamic acid molecules that are deprotonated, while. f_{AH}(r) is the fraction of glutamic acid molecules that are protonated. The variables, f_{ARb}(r), f_{ANa}(r), f_{AK}(r), and f_{ACs}(r) are the fraction of glutamic acids that are condensed with Rb⁺, Nat, K⁺, or Cs+ respectively. Here, μ_{0Ai} is the chemical potential associated with the different chemical states of glutamic acid.

$$\frac{β W}{L} = \int dr A ( r ) {ρ}_{w} ( r ) ( ln {ρ}_{w} ( r ) {v}_{w} - 1 ) + {\sum }_{i = \{ {Na}^{+} , {Rb}^{+} , {K}^{+} , {Cs}^{+} , {Cl}^{-} , {H}^{+} , {OH}^{-} \}} \int dr A ( r ) {ρ}_{l} ( r ) ( ln {ρ}_{i} ( r ) {v}_{w} - 1 + {βμ}_{i}^{0} ) + β \int dr A ( r ) 〈 {ρ}_{Glu} ( r ) 〉 [ {f}_{A -} ( r ) ( ln {f}_{A -} ( r ) + β {μ}_{A -}^{0} ) + {f}_{AH} ( r ) ( ln {f}_{AH} ( r ) + β {μ}_{AH}^{0} ) + {f}_{ANa} ( r ) ( ln {f}_{ANa} ( r ) + β {μ}_{ANa}^{0} ) + {f}_{ARb} ( r ) ( ln {f}_{ARb} ( r ) + β {μ}_{Arb}^{0} ) + {f}_{AK} ( r ) ( ln {f}_{AK} ( r ) + β {μ}_{AK}^{0} ) + {f}_{ACs} ( r ) ( ln {f}_{ACs} ( r ) + β {μ}_{ACs}^{0} ) ] + β \int dr A ( r ) [ 〈 {ρ}_{q} ( r ) 〉 ψ ( r ) - \frac{1}{2} {ϵ}_{0} {ϵ}_{r} ( r ) {( {∇}_{r} ψ ( r ) )}^{2} ] + {\sum }_{i = \{ {A}^{+} , {Na}^{+} , {Rb}^{+} , {Cl}^{-} , {H}^{+} , {OH}^{-} \} ( 17 )} \int dr A ( r ) Δ {u}_{i}^{B} ( r ) {ρ}_{i} ( r ) + β \int dr A ( r ) π ( r ) ( 〈 {ϕ}_{PA} ( r ) 〉 + {\sum }_{l} {ρ}_{l} ( r ) {v}_{l} - 1 ) - {\sum }_{j = \{ {Cl}^{-} , {OH}^{-} \}} {βμ}_{j} \int dr A ( r ) {ρ}_{j} ( r ) - {\sum }_{M = \{ {H}^{+} , {Rb}^{+} , {Na}^{+} , {K}^{+} , {Cs}^{+} \}} {βμ}_{m} ( \int dr A ( r ) [ {ρ}_{M} ( r ) + 〈 {ρ}_{Glu} ( r ) 〉 {f}_{AM} ( r ) ] ) + β \int dr A ( r ) 〈 {ρ}_{Glu} ( r ) 〉 λ ( r ) ) [ {f}_{A -} ( r ) + {f}_{AH} ( r ) + {f}_{ANa} ( r ) + {f}_{ARb} ( r ) - 1 ] \qquad ( S \text{.6} )$$

The next contribution to the free energy expression (fifth line) describes the electrostatic energy, [symbol] ρ_{q}(r) [symbol] which is the total charge number density and ψ(r) corresponds to the electrostatic potential. In the electrostatic energy functional, ϵᵣ(r), corresponds to the relative dielectric constant, which is assumed to be the volume weight average of the dielectric constant of water and the PA-fiber and given by

$${ϵ}_{r} ( r ) = {ϵ}_{w} {ϕ}_{w} ( r ) + {ϵ}_{PA} 〈 {ϕ}_{PA} ( r ) 〉 \approx {ϵ}_{PA} ( 1 - 〈 {ϕ}_{PA} ( r ) 〉 + {ϵ}_{PA} 〈 {ϕ}_{PA} ( r ) 〉 .$$

[symbol]

φ_{PA}(r) [symbol] is the volume fraction of the PA-nanofiber and φ_{w}(r)=ρ_{w}(r)v_{w} corresponds to the volume fraction of water.

The tenth contribution of the free energy, describes the electrostatic solvation energy. Here

$$Δ {u}_{i}^{B} ( r )$$

is the Born or solvation energy associated with all charged species, which is given by

$$Δ {u}_{i}^{B} ( r ) = \frac{{z}_{k}^{2} {e}^{2}}{8 {πϵ}_{0} {a}_{k}} ( \frac{1}{{ϵ}_{r} ( r )} - \frac{1}{{ϵ}_{w}} ) . \qquad ( S \text{.7} )$$

Here, aₖ corresponds to the radius of the ion of type k and zₖ is equal to its valence. The next contribution in free energy, line seven, describes the excluded volume or steric repulsions among all molecular species, which is represented by assuming that the system is incompressible at every position

$$〈 {ϕ}_{PA} ( r ) 〉 + {\sum }_{l} {ρ}_{l} ( r ) {v}_{l} = 1. \qquad ( S \text{.8} )$$

These packing constraints are enforced through the introduction of the Lagrange multipliers or lateral pressures, π(r). The integral of the twelfth- and thirteenth-term account for the fact that the free mobile ions are in equilibrium with a reservoir of given chemical potentials and μₖ is the chemical potential of the molecule of type k. The thirteenth-term integrals account for the total number of protons, and sodium, rubidium, potassium, and cesium ions. The last integral ensures that the number of glutamic acid residues is conserved.

The above-presented free energy functional corresponds to a generalized Poisson-Boltzmann approach. Commonly, the Poisson Boltzmann approach involves the translational entropy of the ions and electrostatic interactions, as described by the second and seventh integral. The subsequent terms in the free energy, usually not considered, describe the effect of charge regulation of the glutamic acid residues, the excluded volume repulsions within the electrolyte solution, the effect of solubility, and varying dielectric constant.

It was observed that free energy, the volume fraction of the PA-nanofiber [symbol] φ_{PA}(r) [symbol] , and the distribution of the Glu number density, [symbol] ρ_{Glu}(r) [symbol] , are fixed and need to be specified separately as input. A complete free energy description of a PA-nanofiber would involve explicit terms for the conformational and translation entropy and interaction energy of the peptide amphiphiles, that are coupled with the free energy term as described above. As the result, the approximation was invoked, and the volume fraction distribution of the PA-nanofiber and the distribution of the glutamic number density was imposed. Thus, it was inferred that those distributions to be fixed and to be unperturbed. Given the high density and hydrophobic nature of the alkyl chain, this is true for the core of the PA nanofiber, this is only approximately true for the peptide part of the PA nanofiber. To obtain the volume fraction distribution of the PA-nanofiber reasonably, predictions from atomistic MD simulations were used. Then, minimization of the above restricted free energy functional determines the amount of charge of the glutamic residues and the amount of ion pairing. It also determines the distribution of solvent and mobile ions distributions, as well as the electrostatic potential. Observed similar approximations have been employed to model the counterion distribution cloud around AU-NP coated with DNA and as well as protein-nucleic acid conjugate.

Minimization of the free energy yields the following expression for the local volume fraction of the solvent

$${ϕ}_{w} ( r ) = {ρ}_{w} ( r ) {v}_{w} = {e}^{- βπ ( r ) {v}_{w}} , \qquad ( S \text{.9} )$$

while the density of the ions reads—

$${ρ}_{k} ( r ) = \frac{1}{{v}_{w}} {e}^{- β ( {μ}_{k}^{0} - {μ}_{k} )} {e}^{- βπ ( r ) {v}_{k}} {e}^{- βψ ( r ) {z}_{k} e} . \qquad ( S \text{.10} )$$

Functional variation of the free energy with respect to the electrostatic potential yields the Poisson equation and its boundary conditions

$$- {ϵ}_{0} {∇}_{r} {ϵ}_{r} ( r ) {∇}_{r} ψ ( r ) = 〈 {ρ}_{q} ( r ) 〉 and - {ϵ}_{0} {ϵ}_{w} \frac{d ψ ( r )}{dr} {\vert}_{r = R} = 0 and {lim}_{r \to ∞} ψ ( r ) = 0. \qquad ( S \text{.11} )$$

Finally, minimization of the free energy with respect to the different charged states of the glutamic acid residue, i.e., f_{A-}(r), f_{AH}(r), f_{ANa}(r), and f_{ARb}(r), yields the following reaction equations:

$$\frac{{f}_{{A}^{-}} ( r )}{{f}_{AH} ( r )} = {e}^{- βΔ {G}_{AH}^{0}} {e}^{- βΔ {G}_{AH}^{solv} ( r )} \frac{{e}^{- βπ ( r ) Δ {v}_{AH}}}{{ρ}_{H} * ( r ) {v}_{w}} , \qquad ( S \text{.12} )$$

$$\frac{{f}_{{A}^{-}} ( r )}{{f}_{AJ} ( r )} = {e}^{- βΔ {G}_{AJ}^{0}} {e}^{- βΔ {G}_{AJ}^{solv} ( r )} \frac{{e}^{- βπ ( r ) Δ {v}_{AJ}}}{{ρ}_{{J}^{+}} ( r ) {v}_{w}} , \qquad ( S \text{.13} )$$

Here the J index corresponds to either Na, Rb, K, or Cs.

ΔG_{0AJ}=μ_{0A-}+μ_{0J+}−μ_{0AJ} is the standard reaction free energy change of either the acid-base equilibrium reaction of the acid or the dissociation equilibrium reaction of the metal-ion pairs: ANa, ARb, AK, or ACs. With, Δv_{AJ}=v_{A}_{_{−}}+v_{H}_{₊}−v_{AJ}, is the difference in volume between the products and reactants. The variable Δ_{GsolvAJ}(r) is a position-dependent ‘solvation’ free energy change and is given by

$$Δ {G}_{AJ}^{solv} ( r ) = Δ {u}_{{A}^{-}}^{B} ( r ) + Δ {u}_{{J}^{+}}^{B} ( r ) - Δ {u}_{AJ}^{B} ( r ) + {E}_{solv} ( r ) ( {v}_{{A}^{-}} - {v}_{AJ} ) , with \qquad ( S \text{.14} )$$

$${E}_{solve} ( r ) = \frac{1}{2} {ϵ}_{0} {ϵ}_{r}^{′} [ {ϕ}_{PA} ( r ) ] {( {∇}_{r} ψ ( r ) )}^{2} + {\sum }_{i = \{ {A}^{+} , {Na}^{+} , {Rb}^{+} , {K}^{+} , {Cs}^{+} , {Cl}^{-} , {H}^{+} , {OH}^{-} \}} {u}_{i}^{B} ( r ) {ρ}_{i} ( r ) \frac{{ϵ}_{r}^{′} [ {ϕ}_{PA} ( r ) ]}{{ϵ}_{r} [ {ϕ}_{PA} ( r ) ]} .$$

In the last line,

$${ϵ}_{r}^{′} [ {ϕ}_{PA} ( r ) ]$$

correspond to the functional derivative with respect to the volume fraction of the PA-nanofiber and u_{Bi}(r)=z₂ₖe₂/8πϵ₀ϵ_{w}aₖ. The solvation free energy contribution vanishes in the case of a uniform dielectric constant. It was observed that the standard free energy change Δ_{G0AJ} is related to the chemical equilibrium (dissociation) constant K_{0AJ}=e_{-βΔG}_{_{0AJ}}, which can be related to the experimental equilibrium constant via K_{AJ}=C e_{-βΔG}_{_{0AJ}} of a single acidic molecule in infinitely dilute solution. Here C is a constant required for consistency of units and equal to C=1/N_{A}v_{w}, where N_{A} is Avogadro's number.

To obtain a solution that needs to be solve the Poisson equation and incompressibility constraint simultaneously. The unknowns of these equations are the lateral pressures, π(r), and the electrostatic potential, ψ(r). Once the lateral pressures and the electrostatic potential have been established, the amount of protonation and ion condensation of the glutamic acids as well as the electrostatic potential and density distribution of the free as well as condensed ions and the solvent are known. The above equations are solved numerically. The required inputs are the reservoir solution conditions (pH and bulk salt concentration of NaCl, RbCl, KCl, and CsCl), the volume of the mobile species (Table 6), the acid-base constant, and ion binding of ion dissociation constants of glutamic acid, (Table 7) and the volume fraction of the PA-fiber as well as the number density of the glutamic acid molecules. The acid-base constant of glutamic acid is equal to that of carboxyl acid. Previously, MD simulations were used to establish the Na-binding equilibrium constant to acetate. The ion dissociation constants of Rb⁺, K⁺, and Cs⁺ with acetate are not known, however, given that Rb⁺, K⁺, and Cs⁺ are larger than Na⁺, the Na binding energy was used as an upper bound and assume the ion dissociation of Rb⁺, K⁺, and Cs⁺ to acetate to be comparable or lower in value.

TABLE 6
Volume mobile (ionic) species

| | w | H+ | OH− | Na+ | K⁺ | Rb+ | Cs⁺ | Cl− |
|---|---|---|---|---|---|---|---|---|
| V(nm³) | 0.03 | 0.03 | 0.03 | 0.0044 | 0.0112 | 0.01471 | 0.01951 | 0.0249 |

TABLE 7
Acid-base chemical equilibrium constant and
ion dissociation constants of glutamic acid.

| reaction | pKₐ |
|---|---|
| AH ⇄ A₋ + H₊ | 5.0 |
| ANa ⇄ A₋ + Na₊ | −0.6 |
| ARb ⇄ A₋ + Rb₊ | <−0.6 |
| AK ⇄ A₋ + K₊ | <−0.6 |
| ACs ⇄ A₋ + Cs₊ | <−0.6 |

### Degree of Dissociation

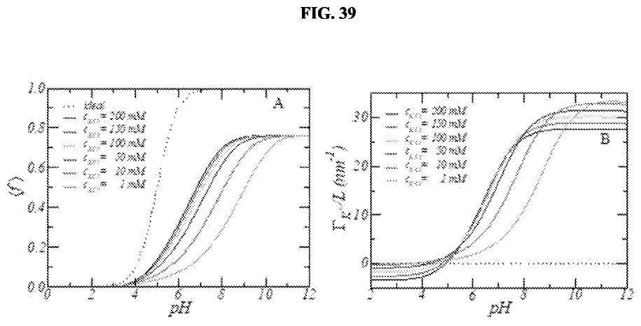

FIG. 39 shows the average degree of dissociation and ion excess per unit length as a function of reservoir pH at different KCl concentrations. The average degree of dissociation is obtained by integration of the position-dependent degree of dissociation f_{A}_{_{−}}(r)

$$〈 f 〉 = {\int }_{0}^{∞} drA ( r ) {f}_{{A}^{-}} ( r ) 〈 {ρ}_{Glu} ( r ) 〉 / {\int }_{0}^{∞} drA ( r ) 〈 {ρ}_{Glu} ( r ) 〉 , \qquad ( S \text{.15} )$$

while the ion excess per unit length of the PA-nanofiber is defined as follows:

$$\frac{{Γ}_{J}}{L} = {\int }_{0}^{∞} drA ( r ) ( {ρ}_{J}^{tot} ( r ) - {ρ}_{J}^{bulk} ) = {\int }_{0}^{∞} drA ( r ) ( {ρ}_{J} ( r ) + {f}_{AJ} ( r ) 〈 {ρ}_{Glu} ( r ) 〉 - {ρ}_{J}^{bulk} ) \qquad ( S \text{.16} )$$

The ion excess measures the excess number of ions that are contained in the ion cloud surrounding the nanofiber and condensed onto the PA-nanofiber relative to the reservoir of bulk ion concentration ρ_{bulkJ}. Here, ρ_{totJ} is the total ion concentration that is the sum of the free and condensed ion concentration. The ion excess can be measured by so-called ion counting measurements or inductively coupled plasma mass spectroscopy (ICPMS). In FIG. 46 it was observed that the titration or the degree of dissociation (i.e., charge) of the PA nanofiber is very different from the amount of charge excepted based on the ideal charging behavior of the same carboxylic acid moiety in dilute solution. Because of the complex balance between various opposing forces, which involve among others the acid-base chemical equilibrium, counter ion confinement, and counter ion condensation as well as the effect of ion solubility, which is coupled with the local dielectric environment inside the PA-nanofiber. It was observed that the degree of charge is far less than anticipated based on ideal solution behavior. The charge is also strongly influenced by the ion concentration, depending on ion concentration only between ~15% and ~57% of the glutamic acid residues of the PA-nanofiber are charged in a KCl solution at pH=7. At elevated pH only about 76% of the glutamic acids are deprotonated and the remaining are found in the ion-condensed state. For intermediate pH also a considerable amount of glutamic acid is to be found in their protonated charge neutral state. It was observed that the ion excess “follows” the titration curve or degree of dissociation as shown in FIG. 46A. With increasing pH the PAs acquire more charge and consequently, more counterion are confined in the ion cloud surrounding the PA and simultaneously more counterion are condensed onto the PA-nanofiber. To emphasize this observation, the ion excess per unit length for both KCl and NaCl solutions. For comparison also the ion excess only considering the free ions is shown along sized the number of ions per unit length that are condensed onto the glutamic acids of the PA-nanofiber. The sum of the “free” ion excess and the total number of condensed ions equals the ion excess. It was observed that a considerable number of counterions were condensed. Also, the amount of ion condensation was different for K⁺ as compared to Na⁺. A “simple” explanation involves noting that Na⁺ is smaller than K⁺ and there needs to be less loss of translation entropy for the ions when confined and condensed onto the PA-nanofiber, simultaneous the solvation or Born energy of Larger for Na⁺ as compared to K⁺ further promoting condensation of Nat. Hence, more Na⁺ is condensed as compared to K⁺.

### Example 5

### Anomalous Small Angle X-Ray Scattering (ASAXS)

### ASAXS Measurements

An anomalous small angle X-ray scattering (ASAXS) was used to deduce the spatial distribution of Rb⁺ surrounding charged, twisted peptide amphiphile (PA) bilayer. ASAXS relies on the measurements of the subtle changes in the X-ray energy-dependent scattered intensity profiles from the combined counterion (here, Rb⁺)-supramolecular assembly system. These changes arise due to strong dispersion corrections [f′(E), f″(E)] to the form factor (scattering/absorption power) of an atom or an ion when the X-ray energy E is close to the binding energy of a core-shell electron in the atom or the ion [(here, Rb⁺ K-edge E_{K}=15.2 keV.

$${f}_{RB} ( q , E ) = {f}_{Rb}^{0} ( q ) + {f}_{RB}^{′} ( E ) + {if}_{RB}^{″} ( E )$$

q=4π sin (θ)λ is the modulus of the scattering vector, θ is one half of the angle between the incident and the scattered X-rays, λ is the X-ray wavelength. In the small angle limit, f_{0Rb}(q) can be taken to be the number of electrons (Z=36) in an Rb⁺ ion. For X-ray energies below the absorption edge, f_{″Rb}~0, and f_{′Rb}(E)<0 accounts for the reduction in the effective number of electrons in the Rb⁺ ion that contribute to the scattering. In this regime, the background-subtracted scattered intensity from counterion-supramolecular assembly system may be written as:

$$ΔI ( q , E ) = \frac{N}{V} [ 〈 {\vert {F}_{0} ( \vec{q} ) \vert}^{2} 〉 + {f}_{RB}^{′} ( E ) 〈 2 {F}_{0} ( \vec{q} ) v ( \vec{q} ) 〉 ]$$

[symbol]

|F₀(\vec{q})|² [symbol] is the non-resonant (X-ray energy-independent) contribution to the scattered intensity from a single twisted PA membrane and the associated Rb⁺. The second non-resonant term contains v(\vec{q}), which is the Fourier transform of the distribution profile of the excess Rb⁺ density [n_{Rb}(\vec{r})−n_{Rb,b}], where n_{Rb,b} is the number density of the Rb⁺ ions in the bulk solution. The multiplicative factor NV represents the concentration of the supramolecular assemblies. The angular brackets represent averaging over all possible orientations of the twisted bilayer and the associated ions. The full form of the ASAXS equation is quadratic in f_{′Rb} due to a third purely resonant term [f_{Rb}′]² [symbol] [v(\vec{q}])² [symbol] . This quadratic term has been neglected because the scattering from just the Rb⁺ ions is expected be to be much weaker than that from the combined chiral twisted membrane-Rb⁺ system.

TABLE 8

| E − Eₖ (eV) | f_{′Rb}(E) |
|---|---|
| −5.1 | −7.59 |
| −13.1 | −6.52 |
| −47.1 | −5.19 |
| −379.1 | −3.14 |

f_{′Rb}(E) at 4 different X-ray energies, which were used in the ASAXS measurements. These f_{′Rb}(E) are based on linear interpolation of the complex scattering factors for Rb tabulated in the Hephaestus software database.

Scattered intensity profiles were measured from 1 mM PA in solutions containing either 66 mM RbCl or 50 mM RbCl and 16 mM OC—Cl at 4 X-ray energies below the Rb⁺ K edge (Table 8). The non-resonant

$$\frac{N}{V} 〈 {\vert {F}_{0} ( \vec{q} ) \vert}^{2} 〉$$

and the counterion distribution-dependent cross-term

$$\frac{N}{V} [ 〈 2 {F}_{0} ( \vec{q} ) v ( \vec{q} ) 〉 ]$$

were separated as intercept and slope of the four ΔI(q,E) vs f_{′Rb}(E) points at each q.

### Parametrized Model for Twisted Ribbon

The coordinates of any point on an infinitely thin twisted ribbon are given by

$$\{ u cos ( φ ) , u sin ( φ ) , T \frac{φ}{2 π} \} .$$

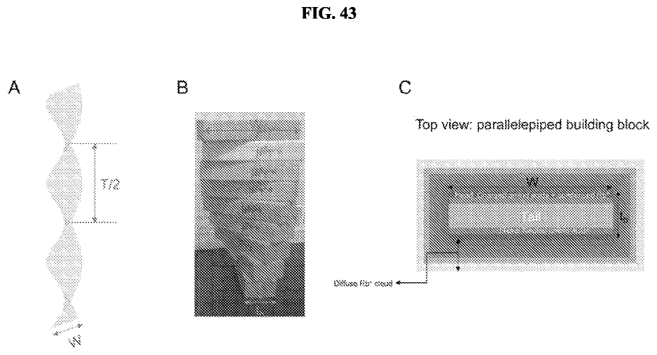

Here, T is twice the separation between the twists in the ribbon (FIG. 43), |u|≤W2 and 0≤φ≤lπ.
Here, W the ribbon width, and l the number of twists in the ribbon. To account for the finite thickness of the bilayer membrane and to create a discretized model for ASAXS analysis, the bilayer membrane is represented via a Lego-like model consisting of parallelepiped blocks (FIG. 43B). Each parallelepiped has a width W, length t, corresponding to the bilayer thickness and a height h. The adjacent blocks are rotated about the ribbon long axis by ψ=πT.

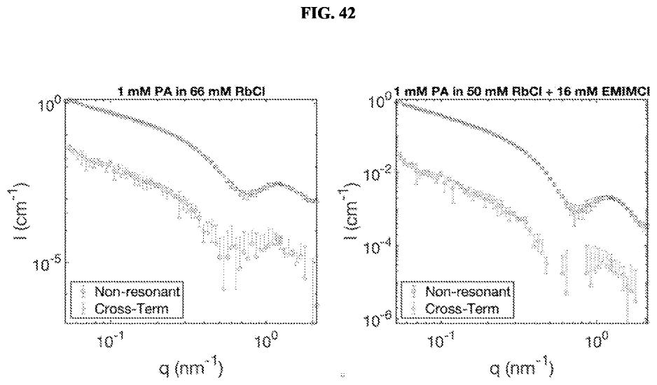

The block height h=1 nm is empirically selected for two reasons. First, no artifacts are introduced by this artificial construct in the simulated scattered intensities over the relevant ASAXS q range of 0.05-2 nm⁻¹ (FIG. 42). Second, this choice keeps the simulations relatively fast. The distribution of Rb⁺ is assumed to follow the shape of each parallelepiped block (FIG. 43C), and consists of the condensed Rb⁺ layer, which is associated with the 2 ionizable amino acid groups in the outer periphery of the PA bilayer, followed by 12 layers with exponentially decaying Rb⁺ concentration. The total number of Rb⁺ associated is equal to the parallelepiped charge, which is calculated based on estimates of the area/molecule/leaflet (Aₜ=0.5 nm²) from molecular dynamics (MD) simulations and a fit parameter for the fraction of amino acids that are in the ionized state (a). Based on this model the orientationally averaged non-resonant intensity and the cross-term are calculated and compared to the data in FIG. 42. The parameters that are varied in these calculations are the electron densities and the thicknesses of the hydrophobic tail region and the hydrophilic head regions: [rₜ, tₜ] and [rₕ, (t_{b}−tₜ)/2], the degree of ionization (a), the bilayer ribbon width W and the fraction of Rb⁺ in the condensed and the diffuse layer. The pitch of the bilayers is derived from AFM and cryo-TEM measurements and is held fixed at T/2=205 nm and 147 nm for the cases where the PA are dispersed in 66 mM RbCl and 50 mM RbCl+16 mM OCCl, respectively.

### ASAXS Analysis

The SAXS profiles in FIG. 42 were analyzed using the discretized model in FIG. 43. Due to the large number of adjustable parameters and orientational averaging, fitting of the data using non-linear least square minimization was extremely slow. To circumvent this, the non-resonant and cross-term profiles over a range of parameters were stimulated. The best-fit was then selected based on manual calculations of the differences between the measured and simulated profiles.

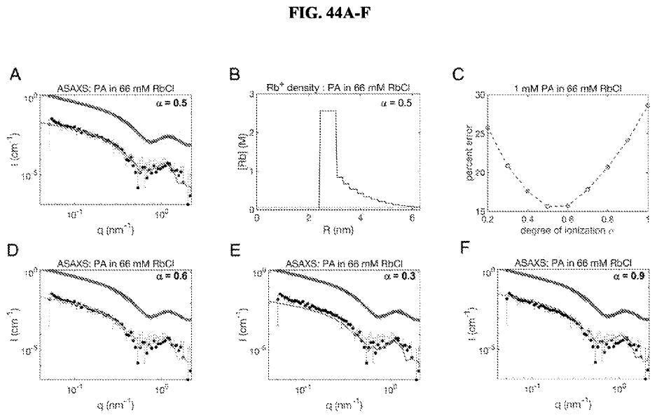

For the case of 1 mM PA in 66 mM RbCl, the best match between the simulated and ASAXS-extracted profiles (FIG. 44A) was found for the case of ribbons with width W=17 nm, bilayer thickness t_{b}=6.1 nm, the thickness of the tail tₜ=2.2 nm and the thickness of the headgroup tₕ=(t_{b}−tₜ)/2=1.95 nm. Furthermore, the degree of ionization was found to be a=0.5. This corresponds to the case where on average only one of the two ionizable amino acids in the PA molecule is in the charged (−1) state. This reduction in ionization tendency maybe expected because an arrangement of charged molecules in proximity is expected to increase the electrostatic potential energy of the assembly. It was noted that the length of the 16 carbon long alkyl tails was (16-1)′0.127~1.9 nm. Therefore, the ASAXS-derived hydrophobic tail region (tₜ=2.2 nm) is much smaller than the length of two C₁₆ alkyl tails (3.8 nm). This shows that the alkyl tails from the two bilayer leaflets are interdigitated. This qualitatively matches the MD simulation results.

According to FIG. 44A, it was shown that the comparison between the ASAXS-extracted non-resonant and the cross-term profiles along with the “best-match” simulation profiles. FIG. 44C explicitly shows that the average difference between the measured and the simulated profiles was minimized for a=0.5. However, the difference in the quality of match is minimal between the cases of a=0.5 and a=0.6 (FIG. 44C-D). For comparison, the poor quality of match between the simulated and the SAXS-extracted cross-terms can be readily seen in FIGS. 44E-F for a=0.3 and a=0.9. The sought-after Rb⁺ concentration profile as a function of distance R from the bilayer center that corresponds to the best-match profiles is shown in FIG. 44B. It was shown that near the negatively charged headgroups, Rb⁺ concentration exceeds the bulk Rb⁺ concentration by ~40×.

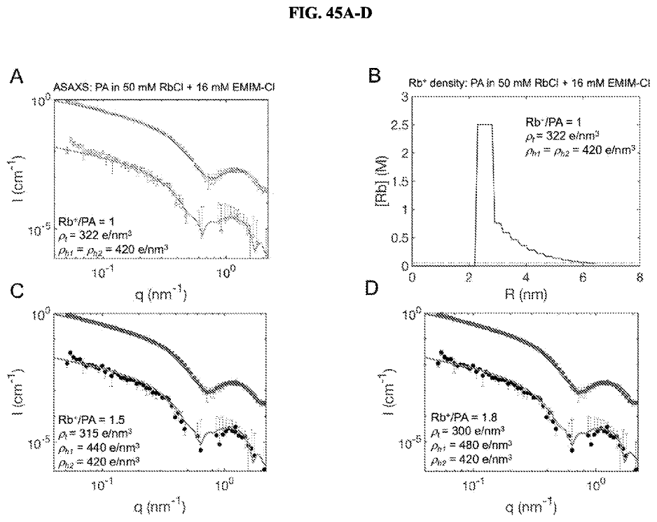

The non-resonant and cross-terms intensities for the case of PA in 50 mM RbCl and 16 mM OC—Cl (FIG. 42, right), were analyzed under the constraint of Aₗ=0.5 nm². This area/lipid/leaflet is identical to the above-described case of PA in 66 mM RbCl. This constraint was imposed because the wide-angle X-ray scattering data from the PA assemblies under the two ionic conditions (66 mM RbCl or 50 mM RbCl+16 mM OC) showed nearly identical diffraction patterns. However, the analysis (FIG. 45) shows that the PA ribbons in 50 mM RbCl+16 mM OC—Cl are narrower in width (W=13 nm) and thinner (t_{b}=5.8 nm) when compared to the case of PA in 66 mM RbCl (W=17 nm, t_{b}=6.1 nm). The decrease in ribbon width is qualitatively consistent with AFM and cryo-TEM measurements. Furthermore, this suggests that the degree of ionization for PA in 50 mM RbCl+16 mM OC exceeds that for the PA in 66 mM RbCl. This is because the enhanced electrostatic interactions in the higher charged membranes are expected to drive the assembly towards a more quasi-1D structure, which reduces, on average, the number of next nearest, next-next nearest molecular neighbors and thereby reduces the electrostatic potential energy of the assembly.

To investigate the degree of ionization in the PA assemblies, the SAXS analysis was done by systematically varying the simulation parameters in 2 ways: (1) the SAXS intensities were simulated by varying the number of Rb⁺/molecule in the counterion layer, while keeping the electron densities for the tail (rₜ) and headgroup regions (rₕ) identical to the best-match values for the case of PA in 66 mM RbCl (rₜ=322 e/nm³ and rₕ=420 e/nm³). Under this approach, the best-match between the simulated and the measured data is obtained for 1 Rb⁺/PA (FIGS. 45A-B). (2) The number of Rb⁺/molecule were fixed at various values (≥1) and the electron density for the tail region and the headgroup region representing the non-ionizable amino acids were allowed to vary. Best-matches between the measured and simulated data obtained for the cases of Rb⁺/PA=1.5 and 1.8 are shown in FIGS. 45C and 45D, respectively.

FIGS. 45A, C-D show that ascertaining the precise Rb⁺/PA is extremely challenging because within experimental uncertainties, and over the relatively small q range of the accessible data, good fits can be obtained for various Rb⁺/PA≥1. This also suggests the need for simplifying the geometric model to make it more amenable to global optimization procedures. Additionally, the above observations imply that (1) the degree of ionization for PA in 50 mM RbCl+16 mM OC is higher than that for PA in 66 mM RbCl. This is because in addition to Rb⁺, some PA charge is expected to be neutralized by OC⁺ in the counterionic layer. Therefore, even for the case of 1Rb⁺/PA, the average PA charge is greater than 1. In other words, the PA degree of ionization a>0.5. (2) The analysis suggests a possible mechanism for the enhanced degree of ionization for PA in 50 mM RbCl+16 mM OC. The findings also show that Rb⁺/PA or the degree of ionization of the PA increases with increasing electron density of non-ionizable headgroup region (rₕₜ) (legends, FIGS. 45C-D). This observation suggests that the hydrophobic OC⁺ ions penetrate the spaces between the nonionizable amino acid blocks on neighboring PA molecules in the membrane. This would increase the electron density in this region. The incorporation of positively charged OC⁺ in between the PA in the membrane enhances the propensity of the glutamic acid groups to be ionized as the interactions between OC⁺ and the negatively charged PA groups would lower the membrane electrostatic potential energy.

Since NMR measurements and MD simulations suggest OC ions displace the water molecules in the non-ionizable peptide region with approximately 8 equivalents of OC molecules per PA, were used as the higher electron density of 480 e/nm³ for ρₕ₁, which corresponds to 1.8 Rb⁺/PA. The geometric fit and resulting Rb⁺ distribution profile is shown in FIGS. 3B-C. The Rb⁺/PA=1.8 implies that 90% of the glutamic acid residues are charged which is in good agreement with NMR measurements that indicate ~100% ionization of the PA when OC is incorporated into the nanostructure.

### Example 6

### Materials and Methods

### Chemicals

All the chemicals were bought and used without any purification from Sigma-Aldrich except anywhere mentioned. Organic Cations (OC) were used without further purification.

### PA Synthesis and Purification

Peptides were synthesized on solid support using a rink amide MBHA resin (100-200 mesh, 0.55 mmol g−1) using standard fluorenylmethyloxycarbonyl (fmoc) procedures. Molecules PA1 were synthesized on Rink amide MBHA resin (aappTEC). Fmoc deprotection was performed using 20% 4-methylpiperidine in DMF for 20 min. The resin was then washed with DMF and swollen with DCM. Amino acid couplings were performed with 4 equivalents of protected amino acid, 4 equivalents of HBTU, and 6 equivalents of DIEA in 50% DMF/50% DCM for 2 h. Palmitic acid was coupled to the peptide N-terminus with 8 equivalents of palmitic acid, 8 equivalents of HBTU, 12 equivalents of DIEA in 50% DMF/50% DCM for 4 h. Deprotection and couplings were verified through ninhydrin colorimetric assays (Kaiser test). This solution was purified using standard preparatory reverse-phase high performance liquid chromatography (HPLC) techniques on a Shimadzu Prominence instrument equipped with a Phenomenex Gemini NX-C18, 30×150 mm column. A mixture of water/acetonitrile containing 0.1% NH4OH was used as eluent. Pure fractions were selected based on the corresponding product signals in electrospray ionization mass spectrometry using direct injection on an Agilent 6520 Q-TOF LC-MS. Organic solvent was removed from selected fractions under reduced pressure before being frozen, lyophilized, and stored at −20° C. until further use. Purity of the samples was determined to be >95% based on the absorption at 220 nm in analytical liquid chromatography-mass spectrometry (LCMS) using a Agilent 1200 system equipped with a Phenomenex Gemini C18, 1×100 mm column with the same Agilent 6520 Q-TOF detector.

### Preparation of OC (X) PAs

After lyophilization, the PA powder was reconstituted in milliQ and adjusted to a pH of 7.4 using 1 μL additions of IN NaOH to ensure cell compatibility and material consistency. ID(X) were added in PAs solution and the mixture sonicated for 20 min in a sonic bath, and the annealed at 80° C. for 30 min and then slowly cooled down at 1° C. per minute to reach a final temperature of 27° C. using a thermocycler (Eppendorf Mastercycler) for even and controlled heating and cooling of all samples. The solutions were lyophilized for an overnight and then reconstituted in 125 mM NaCl and 3 mM KCl solution (saline samples). In the cases of the single ions for the reconstitution was used 150 mM MCI (NaCl, KCl, CsCl, RbCl) and the pH adjustment with the use of the corresponding bases (NaOH, KOH, CsOH, RbOH), in both steps (pH=7.4). PA solutions were then annealed at 80° C. for 30 min and then slowly cooled down at 1° C. per minute to reach a final temperature of 27° C. using a thermocycler (Eppendorf Mastercycler) for even and controlled heating and cooling of all samples.

### Rheology

PA materials were prepared using methods described above. An MCR302 Rheometer (Anton Paar) was used for all rheological studies. PA liquid was placed on the sample stage (150 μL) and 150 mM CaCl₂) solution (30 μL, for a final concentration of 25 mM CaCl₂)) was pipetted onto the underside of a 25 mm cone plate above the material. The instrument stage was set to 37° C. to simulate in vitro culture conditions. The plate was slowly lowered to the measuring position and a humidity collar was used to enclose the sample plunger and prevent sample evaporation during each 45 min experimental run. During the first interval of each experiment, the sample was equilibrated for 30 minutes with a constant angular frequency of 10 [rad/s] and 0.1% strain. The storage and loss modulus (G′ and G″) were recorded at the end of the interval, after a plateau ecured. The angular frequency was incremented from 100 rad/s to 1 rad/s over 21 points. G′ and G″ were recorded for all frequencies. The % strain was increased incrementally from 0.1 to 100% over 31 points and G′ and G″ were recorded.

### Scanning Electron Microscopy (SEM)

PA samples were fixed in a mixture of paraformaldehyde (2.0%, Electron Microscopy Sciences), glutaraldehyde (2.5%, Electron Microscopy Sciences) in phosphate buffered saline (1×, Gibco) for 20 min. The fixative was removed, and the water was exchanged with ethanol by incubating the samples in a gradation of ethanol solutions with increasing concentration (30-100%) of 200 proof ethanol (Decon Laboratories, Inc). Critical drying point (Tousimis Samdri-795) was used to remove the excess water. A purge cycle of 15 min was used. The resulting dehydrated sample coverslips were mounted on stubs using 12 mm carbon adhesive tape (Electron Microscopy Sciences) and coated with approximately 6 nm of osmium (Filgen, OPC-60A) in order to make the sample surface conductive for imaging. All images were taken with an accelerating voltage of 3 kV with a Hitachi SU8030 SEM instrument.

### Cryogenic Transmission Electron Microscopy (CryoTEM)

CryoTEM was performed on a JEOL 1230 at an accelerating voltage of 100 KV equipped with a Gatan 831 CCD camera. PAs were prepared at 1 mM and pipetted at 7.0 μL volumes onto 300-mesh copper grids with lacey carbon support (Electron Microscopy Sciences) that were treated with glow discharge for 20 seconds. Samples were blotted twice at one second per blot before plunging into liquid ethane using a Vitrobot Mark IV (FEI) vitrification robot operating at room temperature and 95-100% humidity. After vitrification, the samples were transferred under liquid nitrogen to a Gatan 626 cryo-holder for imaging.

### Small Angle X-Ray Scattering (SAXS) Measurements

1 wt % PA solutions were loaded in home-built sample cells comprised of two 30 μm thick AS32eco ultrathin glass (Schott AG) as window materials, attached to both sides of a 2.0 mm thick acrylic plate using 9474LE double-stick sheets (3M) as adhesives. The acrylic plates and the double-stick sheets were laser-cut to form sample cells with 6 mm height and 3 mm width, and around 38 μL of the solution was used for each cell. The sealed samples were then mounted on a translational stage at the beamline. Background samples containing buffer solutions were also collected to perform background subtraction. Experiments were performed at the DuPont-Northwestern-Dow Collaborative Access Team (DND-CAT) 5-ID-D station at the Advanced Photon Source (APS), Argonne National Laboratory. Data was collected on a triple area detector system with an X-ray energy at 17 keV. The wavevector q is defined as =(4π/λ) sin (θ/2), where θ is the total scattering angle. Two different spots on each well were measured for one frame at 5 s exposure time, and the data was averaged based on these 3 frames. The acquired 2D scattering data were then reduced to 1D intensity vs. wavevector plots via azimuthal integration around the beam center in GSAS-II software and were subtracted against buffer scattering profile before analysis on small angle power law and Bragg peaks at wide angles were performed. The software SasView 5.0.4 (sasview.org) was used for linear fitting to obtain the slopes or fitting with the core shell parallelepiped model for other detailed structural features.

### Wide-Angle X-Ray Scattering (WAXS)

The measurements were performed at 5ID in the Advanced Photon Source (APS) at the Argonne National Laboratory with a fiber-coupled device (CCD) detector. The wavelength of the incident X-ray was 0.729 Å at an incident energy of 13 keV. 150 μL of sample solution ([PA]=5.3 mM in aqueous NaCl and KCl ([NaCl]=150 mM and [KCl]=3 mM)) was introduced into a glass capillary with a fixed diameter, and X-rays were irradiated over 3 sec. During the irradiation, the sample solution was continuously oscillated using a flow-cell system with a flow-rate of 10 μL/sec.

### Fourier-Transform Infrared (FTIR) Spectroscopy

FTIR spectra of PA samples were recorded on a Bruker model Tensor 37 spectrometer. 100 μL of sample solution ([PA]=1 wt % in D₂O in aqueous NaCl and KCl ([NaCl]=150 mM and [KCl]=3 mM) were placed between two CaF₂ windows with 50 μm separation. The spectra obtained are the result of 25 scans with 1 cm⁻¹ resolution. Additional IR on dried samples used lyophilized powder from 100 μL of sample ([PA]=1 wt % in aqueous NaCl and KCl ([NaCl]=150 mM and [KCl]=3 mM)) placed on an attenuated total reflectance sample stage equipped with a germanium crystal. IR spectra were scanned 32 times using 1 cm⁻¹ resolution and then averaged.

### Circular Dichroism (CD)

Each IKVAV-PA sample was diluted to concentrations between 0.01-0.04 wt % in either H₂O (no salt samples) or buffer containing 150 mM NaCl and 3 mM KCl (high salt). CD spectra were recorded on a JASCO model J-815 spectropolarimeter using a quartz cell of 0.5 mm optical path length. A continuous scanning mode was used with a speed of 100 nm per minute with the sensitivity set to standard mode. High Tension (HT) voltage was recorded for each sample to ensure that the measurement was not saturated. An accumulation of three measurements was used and a buffer sample was background-subtracted to obtain final spectra. The final spectra were normalized to the final concentration of each sample.

### Atomic Force Microscopy (AFM)

Sample solutions were diluted 10× in 150 mM NaCl and deposited on freshly cleaved mica surfaces for ~1 min, and the excess solution was rinsed with 150 mM NaCl. The samples were then rinsed with 150 mM NaCl 20 mM CaCl2 to immobilize the nanostructures on the mica surface, and measurements were performed in the liquid environment. AFM images were captured in PeakForce tapping mode on a Dimension Icon AFM (Bruker) with a silicon nitride cantilever (SNL10-A, Bruker) in a liquid cell. Images were flattened to correct sample tilt before analysis.

### Simulation Procedures

The PAs for the simulations were created in Avogadro and transformed to MARTINI force field CG representation using a modified version of martinize.py (martinize.py, 2.0) to include the aliphatic tail and using coiled coil as choice for secondary structure. The last two E's (furthest from aliphatic tail) and the K are charged while the two first E's are treated as protonated as this was found to be ideal for fiber formation in preliminary simulations. Therefore, the final charge is (−2+1=)−1. Initial structures consist of 300 molecules arranged randomly and spaced a minimum of 3 Å, solvated with CG water and enough ions were added to neutralize the system in a cubic box 21.5×21.5×21.5 nm³. This corresponds to a concentration of 50 mM (7.8, 7.4 and 8.3 wt % for PA1, PA2, PA3, respectively). This is within the range of concentrations commonly used to speed up self-assembly simulations, which can be up to 10 times higher than the experimental systems. All visualizations were rendered using Visual molecular dynamics (VMD). Additionally, bending simulations were set up by placing equilibrated fibers from the previous simulations in a cubic box big enough, 30.0×30.0×30.0 nm³, to ensure that fibers remain as discrete, non-periodic, by not being able to reach opposite walls within the box. This is because periodicity would add unrealistic constrains for the bending of the fibers and this would not be studied reliably.

Coarse grained Molecular Dynamic (CG-MD) simulations were performed in GROMACS 5.0.4, which was also used for the analysis of the simulations. A cut-off of 1.1 nm was used for intermolecular interactions using reaction field with a relative dielectric constant of 15 for electrostatics and potential-shift for Lennard-Jones interactions. All systems were minimized for 5000 steps or until the forces in atoms converged below 2000 pN.

Classical all-Atom Explicit Solvent Molecular Dynamics (MD) Simulations

All-atom MD simulations were performed using the package of GROMACS 2016.3. The modified OPLS-AA force field was employed, which was further optimized for molecules with long hydrocarbon based on the original OPLS-AA force field. The TIP3P-MOD water model was used, which was recommended for the modified OPLS-AA potential. This potential has shown advantages over the original OPLS-AA potential in reproducing experimental properties for long hydrocarbon molecules of density, heat of vaporization, gel-to-liquid-phase transition, chain conformation, diffusion coefficient, viscosity and solvation free energy. The TIP3P-MOD water model resulted in good agreements in terms of the solvation free energies of long hydrocarbon organic molecules. The modified OPLS-AA potential has been employed in investigating amphiphilic surfactants in extracting high valent metal ions, which showed good consistence with the corresponding experimental data. The original OPLS-AA force field parameters were employed for the Na⁺ counterions of peptide amphiphiles and the salt ions of Na⁺ and Cl. The OPLS-AA force field parameters of the EMIM+ and Cl ions have been recently reported by Doherty, et al., where a scaling factor of 0.8 for the atomic partial charges was recommended to reproduce both the experimental structural features and dynamic properties of ionic liquids.

### Molecular Theory to Predict Charge and Ion Distribution Surrounding a PA-Nanofiber

To complement the experiments and simulations, a generalized Poisson-Boltzmann theoretical approach was developed that allows computation of the charge on a PA nanofiber, the distribution of ions that are contained in the cloud surrounding the nanofiber as well as condensed on the fiber as a function of electrolyte conditions. In this theoretical approach, charge regulation of the acid amino-acid groups we considered, which involves the acid-base chemical equilibrium of the glutamic acid residues as well as the possibility of ion condensation. Also, the charge regulation is coupled with the local dielectric environment by allowing for a varying dielectric constant that includes a position-dependent electrostatic solvation energy for all charged species.

### Nuclear Magneti Resonance (NMR) Characterization and Experiments

NMR spectra were acquired at 600 MHz on a Brucker Neo system with QCI-F cryoprobe and Bruker Avance III HD Ascend 800 MHz, 4.0 mm Gradient hr-MAS solid-state probe (¹H, ¹³C, 15N, 2D) (PH HRMAS 800S4 HCND 4G (B5109).

Solution NMR recorded on 1 mM PA concentration in H2O/D2O (9/1 ratio). For the HR-Mas NMR, the samples were prepared in 10 mM concentration, adjusting the pH at pH=7.4, split it in two solutions, and then add to one of it the EMIMCl. After the two steps annealing procedure the solutions were ultracentrifuged and placed in the appropriate crucibles.

### Isothermal Titration Calorimetry (ITC) Release Experiments Protocol

ITC release experiments were performed on a MicroCal ITC 200 (Malvern). Experiments were carried out at 25° C. PA samples (1 mM) were pH adjusted to reach pH 7 and with a final concentration of potassium of 12.5 mM. PA solution was injected in MilliQ water (pH adjusted to 7 right before the measurement) or NaCl solution (12.5 mM) depending on the experiment. The heat release was measured by the calorimeter. The PA was injected over the course of 20 injections 2 μl each, with 4s duration, spacing of 200 and filter set at 5.

Release experiments were only successful with potassium ions. Na, Rb, Cs did not result in consistent data, possibly due to loose ion pairing with the PA (not enough heat upon release) and the differences on the hydration radius.

Fluoresence Recovery after Photobleaching (FRAP) Protocol

FRAP experiments were performed on a Nikon AIR confocal system fitted on an Eclipse Ti-E inverted microscope. A 10× CFI Plan Apochromat VC objective lens with a numerical aperture of X was used to image samples. Data was analyzed using the Fiji package of ImageJ. Experiments were conducted at room temperature (22° C.). PA concentration was kept constant at 1 mM with 1 mol % of fluorescently labelled PA C16V2A2E2K-(Alexa488). All samples were prepared and measured in PBS (1×) at pH 7.4. Coassembly of the fluorescently labelled PA was achieved by rod sonication before the final annealing. Bleaching was performed with a circular spot (7.5 μm radius) using the 488-laser line operating at 100% laser power. A single iteration was used for the bleach pulse, which lasted 1 s. Fluorescence recovery was monitored at low laser intensity (0.2%) at 2 s intervals. Three separate FRAPs were performed and then averaged to generate a single FRAP curve. The curves were fitted using the relation:

$$frap ( t ) = {e}^{\frac{{t}_{d}}{2 t}} ( {l}_{0} ( \frac{{t}_{d}}{2 t} ) + {l}_{1} ( \frac{{t}_{d}}{2 t} ) )$$

t_{d} was extracted from the fit and used to calculate the effective diffusion D_{eff}

### Ionic Conductivity

Electrochemical impedance spectroscopy was performed on a Metrohm Autolab PGSTAT128N potentiostat equipped with a FRA32M module. Measurements were made on thin gels of PA and OC-PA coated on glass coverslips to assess the charge transfer properties of the materials in the same environment as the in vitro cell culture studies. To prepare coverslips for electrical measurements, 50 nm thick Au electrodes were thermally evaporated onto 15 mm diameter glass coverslips, with 6.5 mm channel between electrodes. The coverslips were incubated in 1×PDL for 1 hour at 40° C., then washed twice with milli-Q water, and dried gently with compressed N₂ gas. 12 μL of 1 wt % PA or OC-PA solution was dropcast on a parafilm sheet and the electrode-coated coverslip was inverted on top of the droplet and allowed to dry overnight at RT. The coverslips were then peeled from the parafilm and washed twice with PBS to rehydrate the hydrogel film. Electrical leads were attached to each electrode and a 100 μL droplet of 1×PBS was placed on top of the hydrogel film to maintain hydration and mimic the cell culture environment during the measurement. AC impedance spectroscopy measurements were performed at 0 V vs. counter in a two-electrode setup with a 10 mV perturbation and frequencies ranging from 100 kHz to 0.1 Hz.

### Electrophoretic Mobility

Electrophoretic mobility and size distribution measurements were recorded on a Malvern Zetasizer Nano ZSP spectrometer directly after bath sonication of each sample for 30 min to break up long fibers and decrease the effects of anisotropy and different viscosity solutions.

### Animals

All animal housing and procedures were performed in accordance with the Public Health Service Policy on Humane Care and Use of Laboratory Animals. All procedures were approved by the Northwestern University Institutional Animal Care and Use Committee. Timed pregnant (E16.5) CD1 mice were supplied by Charles River Laboratories (Wilmington, MA).

### Embryonic Cortical Neuron Cultures

Neurons were obtained from embryonic brains using standard procedures. Time-pregnant mouse was sacrificed by cervical dislocation and the embryos were extracted at embryonic day 16 (E16.5). Cerebral cortices were dissected from the mouse embryos and meninges were removed in a solution of Hank's Balanced Salt Solution (HBSS) with 1% pen-strep (Invitrogen) and then digested with trypsin (Invitrogen) and DNAse (Sigma-Aldrich) for 10 min at 37° C. The tissue was mechanically dissociated, centrifuged at 1000 g for 5 min, and resuspended in CO2-equilibrated Neurobasal (NB, Invitrogen) neuronal culture medium supplemented with 10% normal horse serum (NHS, Invitrogen), 1% pen-strep (Invitrogen), 0.5 mM L-glutamine (Invitrogen), and 5.8 μL NaHCO/mL (Sigma-Aldrich). The cell suspension was pre-plated at 37° C. for 30 min. The pellet was resuspended in NB neuronal culture medium (1% NHS, 1% pen-strep, 0.5 mM L-glutamine, 22 μM glutamic acid (Sigma-Aldrich), 2% B27 (Gibco), and 5.8 μL NaHCO₃/mL (Sigma-Aldrich), and plated at different densities (depending on the type of experiment, see below) directly on tissue culture plates coated with poly-D-lysine (Sigma-Aldrich). After 24 hrs, the medium was replaced with serum-free neuronal culture medium (1% pen-strep, 0.5 mM L-glutamine, 2% B27, 5.8 μL NaHCO₃/mL).

### Embryonic Cortical Neuron Cultures Seeking on PA or OC-PA Coatings

Neuronal cells were plated on poly D-lysine surfaces coated with various PA coatings: PA (C₁₆V₂A₂E₂), OC-PAs coatings; PA mixed 1-Ethyl-3-methyl imidazolium chloride (OC1), 1-ethyl-pyridinium chloride (OC2), 1-ethyl-1-methylpyrrolidinium chloride (OC3), trimethylsulfonium chloride (OC4), tetramethylammonium chloride (OC5). Cells were cultured in 12 or 24 well plates at a density of 400,000 or 50,000 cells/well respectively, for different time points (2 days, 1, 2 and 3 weeks). Samples were fixed in 4% PFA for 15 min (20 min for gels) at room temperature (RT) for immunofluorescence studies or cell extracts were obtained for western blot.

### Western Blotting

For Western blot analysis, protein extracts were obtained from primary cultures after 7, 14 or 21 DIV, and total protein extracts were separated by SDS-polyacrylamide gel electrophoresis and electro-transferred to PVDF membranes. Membranes were blocked with 5% bovine serum albumin (BSA, Sigma-Aldrich) and incubated first with primary antibodies overnight at 4° C., and then with their corresponding secondary HRP-conjugated antibodies (1:5000; ThermoScientific). Protein signals were detected by the ECL chemiluminescent system (Azure Biosystems). Densitometry analysis, standardized to β-Actin as control for protein loading, was performed with ImageJ software (National Institutes of Health, USA). The analyses were averaged from 2-3 independent experiments.

### Immunocytochemistry

For immunofluorescence, neurons were incubated with primary antibodies overnight at 4° C., and then with their appropriate Alexa-488 or Alexa-594 conjugated secondary antibodies (1:500, Thermofisher). DAPI (1:1000, Thermofisher) was used to counterstain nuclei. Finally, the preparations were cover-slipped with ImmunoMount or VectaShield (Thermo Scientific). The following primary antibodies were used for Western blot and/or Immunocytochemistry; Tuj-1 (neuronal marker, 1:100, Biolegend), GFAP (astrocytes marker, 1:2000, Dako), GABA (neuronal marker 1:10000, Abcam), NeuN (neuronal marker 1:10000, Millipore), MAP-2 (neuronal marker 1:10000, Millipore), Gapdh (cytoskeleton marker, 1:1000, Cell signaling), PSD95 (synaptic marker, 1:1000, NeuroMab), Synaptophysin (synaptic marker, 1:500, Abcam), Actin (cytoskeleton marker, 1:1000, Sigma-Aldrich) and Tubulin (cytoskeleton marker, 1:1000, Millipore).

### Cell Viability

For cell viability experiments, media was exchanged with PBS containing 2 μM calcein-AM (Life Technologies) and 100 ng ml 1propidium iodide (Sigma Aldrich) for 20 min at 37° C. The cells were then rinsed with PBS and imaged with a Nikon A1R confocal laser-scanning microscope with GaAsP detectors. Live and dead cells were counted using the Cell Counter plug-in for ImageJ.

### Imaging/Analysis of Cells

Fluorescent preparations were viewed and micrographs were captured with a Nikon AIR confocal laser-scanning microscope with GaAsP detectors or a Nikon Ti2 Widefield Microscope. For in vitro cells quantification, image files were imported into NIH Image J (1.51) software and the “analyze particles” and “cell counter” functions were used to measure the total number of cells in a determined area. Images were assembled in Adobe Photoshop (v. 7.0), with adjustments for contrast, brightness, and color balance to obtain optimum visual reproduction of data. The analyses were performed using ImageJ software (National Institutes of Health, USA).

### IncuCyte Live Imaging

Cell were plated on 12 well plate coated with PAs or OC-PAs. Immediately after seeding the cells, plates were imaged every hour for 6 days in vitro using the real-time quantitative live-cell imaging IncuCyte® (20× objective). Neurites were false colored in purple and analyzed using the automatic IncuCyte® Live-Cell Analysis System.

### Mouse Embryonic Cortical Neuron Patch Clamp Electrophysiology

For electrophysiology experiments, mouse cortical neurons were plated on coverslips at a density of 30,000 cells per well. Whole-cell current-clamp recordings were made from visually identified primary cultured neurons on day 12-14 in culture using inverted Olympus IX51 microscope equipped with a 40× objective. Recording pipettes were made of glass capillaries using a horizontal Sutter P-1000 puller yielding a 3-5 MΩ resistance pipette when filled with standard intracellular solution containing (in mM): 120 K-MeSO₄, 10 KCl, 10 HEPES, 10 Na2-phosphocreatine, 4 Mg-ATP, 0.4 Na₃-GTP, pH 7.3; 285-290 mOsm. Neurons cultures on PA coatings were continuously perfused with standard oxygenated aCSF bath solution (in mM): 125 NaCl, 26 NaHCO₃, 2.5 KCl, 1.25 NaH₂PO₄, 1 MgSO₄, 25 glucose, 2 CaCl₂), pH 7.4 at 32-35° C.; osmolality 310-315 mOsm/Kg. Whole-cell current-clamp data was acquired using an Multiclamp 200B amplifier (Molecular Devices, USA) and digitized at 10 kHz (filtered at 3 kHz) with the neurons held at −65 mV. Resting membrane potential was measured immediately after breaking into the cell. Input resistance was calculated as the slope of the voltage-current curve using 500 ms current steps from −50 pA to 30 pA at 10 pA steps. OC-PA+NBQX neurons had significantly larger input resistance compared to IPDA neurons. AP amplitudes, thresholds, half-widths and fAHP measures were taken from ramp current injection steps (50-400 pA, 500 ms). AP threshold was calculated where the first derivative of the up phase of the trace equals 20 mV/ms. Neurons meeting our quality criteria were used: series resistance <20 MΩ, membrane resistance >100 MΩ, resting potential <−40 mV, and AP amplitude >65 mV from holding. Data was analyzed using MATLAB protocols designed specifically for these experiments. Neurons were chronically treated with 5 μM NBQX starting at 2 DIV.

### Multi Electrode Array (MEA) Plates

12-well MEA plates with 64 electrodes per well were coated with PDL and various PA coatings. Embryonic cortical neurons were seeded at a density of 60,000 cells/well and cultured during 28 DIV. On day 2, cells were treated with NBQX (5 μM) or starvation media. Every 4 days, half of the media was removed from each well and replaced with fresh media containing additional treatment. Spontaneous network and synchronized activity were recorded using Axion Biosystems Maestro 768 channel amplifier and Axion Integrated Studios (AxIS) v2.4 software from day 10 to 28 in vitro. The amplifier recorded from all channels simultaneously using a gain of 1200× and a sampling rate of 12.5 kHz/channel. After passing the signal through a Butterworth band-pass filter (300-5000 Hz) on-line spike detection (threshold=6× the root-mean-square of noise on each channel) was done with the AxIS adaptive spike detector. All recordings were conducted at 37° C. with appropriate 5% CO2/95% 02. Spontaneous network activity was recorded for 5 min each day starting at day 10. Active electrodes were defined as having >5 spikes/min and only wells with over 10 active electrodes during the baseline-recording period were used in the analysis. The percentage of spikes occurring within bursts (%), number of network bursts and synchrony index were used as a measure of neuronal network activity as this demonstrates maturity of neuronal functional properties. All data reflects well-wide averages, where the reported value of n represents the number of wells per condition.

### In Vitro Cortical Neuron Statistical Analysis

Data was analyzed using GraphPad Prism software (version 5.04). Comparisons between pairs of experimental groups were performed using Student's t-test. Comparisons among three or more groups were conducted using ANOVA with Tukey's Multiple Comparison post-hoc test or

ANOVA with Bonferroni's post-hoc test. All data was presented as mean±standard error of the mean (SEM) unless otherwise noted. For in vitro studies, all bar graphs are overlaid with dot plots where each dot represents the value for one in vitro sample or well. All in vitro experiments were repeated independently with at least three times with three replicas' per condition with similar results.

### Organotypic Hippocampal Slice Cultures and PAs/OC-PAs Application

All experiments were carried out according to the European and institutional guidelines for the care and use of laboratory animals (Council Directive 86/609/EEC and French National Research Council) and approved by the local health authority (Préfecture des Bouches-du-Rhône, Marseille). Young wild type Wistar rats (P5-P10), of either sex, were used to prepare organotypic hippocampal slice cultures. Animals were deeply anesthetized with isoflurane (5%) and killed by decapitation. The brain was removed and both hippocampi were individually dissected. Hippocampal slices (300 μm) were cut using a Vibratome (Leica, VT1200S, Wetzlar, Germany) in a sucrose based cutting solution kept at 3-4° C. containing for 1L: ultrapure water (988.5 mL), sucrose (97 g), NaHCO3 (2.18 g), D-glucose (1.8 g), KCl (0.1 g), CaCl2 1M (1 mL), MgCl2 1M (10 mL). Kynurenic acid and penicillin/streptomycin were added to the solution just before cutting to control cellular electrical activity and avoid contamination during slices preparation respectively. Only dorsal hippocampal slices were collected and placed on 20 mm latex membranes (Millicell, Millipore) inserted into 35 mm Petri dishes containing 1 mL of culture medium and maintained in an incubator at 34° C., 95% 02-5% CO2. The culture medium contained 25 mL MEM, 1.25 mL HBSS, 12.5 mL horse serum, 0.5 mL penicillin/streptomycin, 0.8 mL glucose (1 M), 0.1 mL ascorbic acid (1 mg/mL), 0.4 mL Hepes (1 M), 0.5 mL B27, and 8.95 mL sterile H2O. At least 1 h before collecting the slices, PAs control, OC-PA KCl or OC-PA NaCl were painted on the external surface of the latex membranes by dragging a pipette (50 μL of annealed PAs control or OC-PAs) to extrude a thin coat of material and let organotypic slice cultures grow on it. The culture medium was changed regularly (every 3 days). Organotypic hippocampal slice cultures were used at 6, 10 and 14 DIV to perform electrophysiological recordings and at 10 DIV to perform immunostainings.

### Organotypic Hippocampal Slice Culture Electrophysiological Recordings

To perform electrophysiological recordings, the Millicell membrane containing the slices treated with PAs or OC-PAs was cut with a scalpel and transferred to a recording chamber perfused with oxygenated artificial cerebrospinal fluid (aCSF). This solution was maintained at 31° C. and contained: 125 mM NaCl, 26 mM NaHCO₃, 3 mM CaCl₂, 2.5 mM KCl, 2 mM MgCl₂, 0.8 mM NaH₂PO₄, 10 mM D-glucose. It was finally equilibrated with 95% O2-5% CO2. Neurons were visualized under an upright microscope (Olympus BX50WI) in phase contrast by infrared video-microscopy using a 60× immersion objective. Whole-cell patch-clamp recordings were obtained from CA1 pyramidal neurons in dorsal hippocampal organotypic cultures at DIV6,10 and 14. Patch-clamp pipettes (7-9 MΩ) were filled with a solution containing (mM): K-gluconate 120, KCl 20, HEPES 10, EGTA 0, MgCl₂ 2, Na₂ATP 2, and NaGTP 0.3 (pH 7.4). CA1 pyramidal neurons were recorded in whole cell current clamp with a MultiClamp 700B amplifier (Axon Instruments), digitalized at 20 kHz with Digidata 1440A (Molecular Devices), low pass filtered at 10 kHz, and acquired with Clampex software (Molecular Devices). For current-clamp recordings, pipette capacitance was compensated, and the bridge was balanced. Apparent input resistance was tested by current injection (−50 pA, 1000 ms). Input resistance was determined by the subtraction of the steady-state voltage change during hyperpolarizing current injection from the baseline. Experiments were omitted if input resistance changed by >±15% in the same cell recording. All cells were held at approximately −65 mV during all experiments. Input-output curves corresponding to the number of action potential elicited by each increment of injected current (from −50 to 240 pA by increments of 10 pA) were determined for each neuron and allowed to examine the following parameters: the rheobase (the minimal current eliciting at least one action potential), the intrinsic excitability (number of action potentials elicited), the input resistance (a measure reflecting the extent to which membrane channels are open), the afterhyperpolarization (the hyperpolarizing phase of a neuron's action potential), the threshold and amplitude of the action potential.

### Organotypic Hippocampal Slice Culture Electrophysiological Data Analysis

All data analyses were performed with custom written software in Igor Pro 6 (Wavemetrics, Lake Oswego, OR, USA). AP threshold was determined from the first AP evoked by a near-rheobase current. Afterhyperpolarization potentials (AHPs) were defined as the minimum voltage between APs and measured at intermediate current steps. Current amplitudes were calculated as the average of the last 100 ms of intermediate currents steps. Pooled data are presented as mean±SE and statistical analysis was performed using Prism GraphPad. Figures were prepared for presentation using Adobe Illustrator.

### Immunofluorescence of Organotypic Hippocampal Slice Cultures

To perform immunostainings, 10 DIV organotypic slices treated with PAs control or OC-PAs were rinsed in PBS at 37° C. and immediately immersed in ice-cold 4% paraformaldehyde in PBS. Slices were fixed at 4° C. for 15 minutes and subsequently incubated in PBS containing 50 mM NH₄Cl (15 min, RT) to quench the remaining free aldehyde groups. Afterwards, the Millicell membrane containing the slices was cut with a scalpel, and the slices were blocked and permeabilized (O/N, 4° C.) in PBS containing 0.5% Triton X-100 and 5% Normal Goat Serum (NGS Jackson ImmunoResearch). Then, slices were incubated with the primary antibodies (24 hrs, 4° C.) in a solution containing 0.5% Triton X-100 and 2% NGS in PBS. The following primary antibodies were used in these experiments: guinea pig anti-AnkG (1:500, 386005, RRID: AB_2737033 from Synaptic Systems), rabbit anti-Naᵥ1.6 (1:200, Thermo Scientific), rabbit anti-pCreb (1:500, Cell Signaling). Afterwards, sections were washed three times (20 min each) in PBS containing 0.5% Triton X-100 and then incubated with the appropriate secondary antibodies (2h, RT) in PBS containing 0.5% Triton X-100 and 2% NGS. The following secondary antibodies were used in these experiments: goat anti-guinea pig Alexa Fluor 647 (1:150, Jackson ImmunoResearch), goat anti-rabbit Alexa Fluor 488 (1:600, Jackson ImmunoResearch), goat anti-rabbit Alexa Fluor 647 (1:300, Jackson ImmunoResearch). Finally, sections were washed three times (20 min each) in a PBS solution containing 0.5% Triton X-100, then incubated with DAPI (1.5 μg/mL in PBS, Sigma-Aldrich) for 10 min, washed one last time in PBS and mounted in Vectashield Antifade Mounting Medium (Vector laboratories). Slides were kept at 4° C. until use.

### Image Acquisition and Analysis

Images were acquired on a Zeiss LSM-780 Confocal scanning microscope. All experiments images were analyzed with ImageJ. All images from the same day of experiment were taken at the same parameters (gain, laser power). For the analysis of Nav1.6 expressions at the AIS, several ImageJ plugins were used. Briefly, z-stacks of 10 optical slices for each experimental condition were acquired with a 63× objective in the hippocampal CA1 subregion. Ankyrin G signal was used to detect 3D objects with the 3D objects counter plugin (PMID: 17210054). The objects were then added to the 3D ROI manager and a 3D binary mask was created. The ROIs in this binary mask were processed with binary operators (Dilate and Fill holes) and the selection mask was over-imposed to the Nav1.6 z-stacks. The Nav 1.6 signal ascribable to the AIS was quantified as average grey values with the 3D ROI manager and normalized to the PA experimental condition. For the pCREB analysis two different methods were used: i) percentage of pCREB⁺ cells, and ii) fluorescent analysis. First, z-stacks of 10 optical slices for each experimental condition were acquired with a 25× and 63× objectives in the hippocampal CA1 subregion. To calculate the percentage of pCREB positive cells, DAPI was used to detect 3D objects with the 3D objects counter plugin. The objects were then added to the 3D ROI manager and a 3D binary mask was created. The ROIs in this binary mask were processed with binary operators (Dilate and Fill holes) and the total number of ROIs was considered the number of total cells. The selection mask was over-imposed to the pCREB z-stacks. The pCREB positive cells were calculated as the percentage of detected pCREB ROIs over the total number of cells. Finally, the pCREB signal was quantified as average grey values with the 3D ROI manager and normalized to the PA condition.

### Statistics in Rat Slice Experiments

Pooled data are presented as means±SEM. Statistical comparisons were made using Mann-Whitney test with Prisma GraphPad software. Differences were considered significant at p <0.05 (*p<0.05; **p<0.005; ***p<0.0005).

### REFERENCES

The following references, some of which are cited above by number, are herein incorporated by reference in their entireties.
Q. Zhao, S. Stalin, C.-Z. Zhao, L. A. Archer, Designing solid-state electrolytes for safe, energy-dense batteries. Nature Reviews Materials 5, 229-252 (2020).
D. Holeman, R. Yuste, The new nanophysiology: regulation of ionic flow in neuronal subcompartments. Nature Reviews Neuroscience 16, 685-692 (2015).
J. Maier, Nanoionics: ion transport and electrochemical storage in confined systems. Nature Materials 4, 805-815 (2005).
M. J. Langton, Engineering of stimuli-responsive lipid-bilayer membranes using supramolecular systems. Nature Reviews Chemistry 5, 46-61 (2021).
L. K. Kaczmarek, Non-conducting functions of voltage-gated ion channels. Nature Reviews Neuroscience 7, 761-771 (2006).
Ehud Y. Isacoff, Lily Y. Jan, Daniel L. Minor, Conduits of Life's Spark: A Perspective on Ion Channel Research since the Birth of Neuron. Neuron 80, 658-674 (2013).
S. G. Waxman, Transcriptional channelopathies: An emerging class of disorders. Nature Reviews Neuroscience 2, 652-659 (2001).
S. G. Waxman, Channel, neuronal and clinical function in sodium channelopathies: from genotype to phenotype. Nature Neuroscience 10, 405-409 (2007).
T. S. Cohen, A. Prince, Cystic fibrosis: a mucosal immunodeficiency syndrome. Nature Medicine 18, 509-519 (2012).
J. Payandeh, M. Volgraf, Ligand binding at the protein-lipid interface: strategic considerations for drug design. Nature Reviews Drug Discovery 20, 710-722 (2021).
K. Kaila, T. J. Price, J. A. Payne, M. Puskarjov, J. Voipio, Cation-chloride cotransporters in neuronal development, plasticity and disease. Nature Reviews Neuroscience 15, 637-654 (2014).
Y. Zhang et al., A microscale soft ionic power source modulates neuronal network activity. Nature 620, 1001-1006 (2023).
X. Wu, E. N. W. Howe, P. A. Gale, Supramolecular Transmembrane Anion Transport: New Assays and Insights. Accounts of Chemical Research 51, 1870-1879 (2018).
F. De Riccardis, I. Izzo, D. Montesarchio, P. Tecilla, Ion Transport through Lipid Bilayers by Synthetic Ionophores: Modulation of Activity and Selectivity. Accounts of Chemical Research 46, 2781-2790 (2013).
J. Comtet et al., Nanoscale capillary freezing of ionic liquids confined between metallic interfaces and the role of electronic screening. Nature Materials 16, 634-639 (2017).
A. J. Simon et al., Supercharging enables organized assembly of synthetic biomolecules. Nature Chemistry 11, 204-212 (2019).
F. Tantakitti et al., Energy landscapes and functions of supramolecular systems. Nature Materials 15, 469-476 (2016).
J. R. Wester et al., Supramolecular Exchange among Assemblies of Opposite Charge Leads to Hierarchical Structures. Journal of the American Chemical Society 142, 12216-12225 (2020).
S. Srivastava et al., Gel phase formation in dilute triblock copolyelectrolyte complexes. Nature Communications 8, 14131 (2017).
R. M. Capito, H. S. Azevedo, Y. S. Velichko, A. Mata, S. I. Stupp, Self-Assembly of Large and Small Molecules into Hierarchically Ordered Sacs and Membranes. Science 319, 1812-1816 (2008).
K. E. Inostroza-Brito et al., Co-assembly, spatiotemporal control and morphogenesis of a hybrid protein-peptide system. Nature Chemistry 7, 897-904 (2015).
M. J. Webber, E. A. Appel, E. W. Meijer, R. Langer, Supramolecular biomaterials. Nature Materials 15, 13-26 (2016).
V. Rangroo Thrane et al., Ammonia triggers neuronal disinhibition and seizures by impairing astrocyte potassium buffering. Nature Medicine 19, 1643-1648 (2013).
M. H. Sangji et al., Supramolecular Interactions and Morphology of Self-Assembling Peptide Amphiphile Nanostructures. Nano Letters, (2021).
N. A. Sather et al., 3D Printing of Supramolecular Polymer Hydrogels with Hierarchical Structure. Small 17, 2005743 (2021).
P. Perez-García et al., Refinement of Active and Passive Membrane Properties of Layer V Pyramidal Neurons in Rat Primary Motor Cortex During Postnatal Development. Frontiers in Molecular Neuroscience 14, (2021).
J. Liu et al., Genetically targeted chemical assembly of functional materials in living cells, tissues, and animals. Science 367, 1372-1376 (2020).
B. Guo, P. X. Ma, Conducting Polymers for Tissue Engineering. Biomacromolecules 19, 1764-1782 (2018).
J. G. Roth et al., Advancing models of neural development with biomaterials. Nature Reviews Neuroscience 22, 593-615 (2021).
R. J. Nap et al., Acid-Base Equilibrium and Dielectric Environment Regulate Charge in Supramolecular Nanofibers. Frontiers in Chemistry 10, (2022).
K. Krishnamoorthy et al., Defining the Structure of a Protein-Spherical Nucleic Acid Conjugate and Its Counterionic Cloud. ACS Central Science 4, 378-386 (2018).
Y. Sanada et al., Hydrophobic Molecules Infiltrating into the Poly(ethylene glycol) Domain of the Core/Shell Interface of a Polymeric Micelle: Evidence Obtained with Anomalous Small-Angle X-ray Scattering. Journal of the American Chemical Society 135, 2574-2582 (2013).
Y. Wang et al., Double helical conformation and extreme rigidity in a rodlike polyelectrolyte. Nature Communications 10, 801 (2019).
P. Porion, A. Delville, Multinuclear NMR study of the structure and micro-dynamics of counterions and water molecules within clay colloids. Current Opinion in Colloid & Interface Science 14, 216-222 (2009).
K. Sakamoto, K. Karelina, K. Obrietan, CREB: a multifaceted regulator of neuronal plasticity and protection. Journal of Neurochemistry 116, 1-9 (2011).
M. Sheng, G. McFadden, M. E. Greenberg, Membrane depolarization and calcium induce c-fos transcription via phosphorylation of transcription factor CREB. Neuron 4, 571-582 (1990).
X. Cecchi, D. Wolff, O. Alvarez, R. Latorre, Mechanisms of Cs+ blockade in a Ca2+-activated K+ channel from smooth muscle. Biophysical Journal 52, 707-716 (1987).
L. J. Cox, U. Hengst, N. G. Gurskaya, K. A. Lukyanov, S. R. Jaffrey, Intra-axonal translation and retrograde trafficking of CREB promotes neuronal survival. Nature Cell Biology 10, 149-159 (2008).
M. S. Cembrowski, N. Spruston, Heterogeneity within classical cell types is the rule: lessons from hippocampal pyramidal neurons. Nature Reviews Neuroscience 20, 193-204 (2019).
K. Sugino et al., Mapping the transcriptional diversity of genetically and anatomically defined cell populations in the mouse brain. eLife 8, e38619 (2019).
M. S. Fanselow, H.-W. Dong, Are the Dorsal and Ventral Hippocampus Functionally Distinct Structures? Neuron 65, 7-19 (2010).
D. Debanne, E. Campanac, A. Bialowas, E. Carlier, G. Alcaraz, Axon Physiology. Physiological Reviews 91, 555-602 (2011).
J. Shao et al., MAPK-ERK-CREB signaling pathway upregulates Naᵥ1.6 in oxaliplatin-induced neuropathic pain in the rat. Toxicology Letters 384, 149-160 (2023).
M. J. Thrippleton, J. Keeler, Elimination of Zero-Quantum Interference in Two-Dimensional NMR Spectra. Angewandte Chemie International Edition 42, 3938-3941 (2003).
J. H. Ortony et al., Internal dynamics of a supramolecular nanofibre. Nature Materials 13, 812 (2014).
D. E. Woessner, NMR relaxation of spin-3/2 nuclei: Effects of structure, order, and dynamics in aqueous heterogeneous systems. Concepts in Magnetic Resonance 13, 294-325 (2001).
W. D. Rooney, C. S. Springer Jr, A comprehensive approach to the analysis and interpretation of the resonances of spins 3/2 from living systems. NMR in Biomedicine 4, 209-226 (1991).
F. Cesare Marincola, M. Casu, G. Saba, A. Lai, 23Na NMR Relaxation Studies of the Na-DNA/Drug Interaction. ChemPhysChem 2, 569-575 (2001).
E. Deiss-Yehiely, J. H. Ortony, B. Qiao, S. I. Stupp, M. Olvera de la Cruz, Ion condensation onto self-assembled nanofibers. Journal of Polymer Science Part B: Polymer Physics 55, 901-906 (2017).
R. J. Nap et al., Acid-Base Equilibrium and Dielectric Environment Regulate Charge in Supramolecular Nanofibers. Frontiers in Chemistry 10, (2022).
B. Hess, C. Kutzner, D. van der Spoel, E. Lindahl, GROMACS 4: Algorithms for Highly Efficient, Load-Balanced, and Scalable Molecular Simulation. Journal of Chemical Theory and Computation 4, 435-447 (2008).
F. Jiménez-Ángeles, K. Harmon, T. D. Nguyen, P. Fenter, M. O. de la Cruz, Non-Reciprocal Interactions Induced by Water in Confinement. Phy. Rev. Res. 2, 043244 (2020).
B. Qiao, F. Jiménez-Angeles, T. D. Nguyen, M. Olvera de la Cruz, Water follows polar and nonpolar protein surface domains. Proc Natl Acad Sci USA 116, 19274-19281 (2019).
M. Deserno, F. Jiménez-Angeles, C. Holm, M. Lozada-Cassou, Overcharging of DNA in the Presence of Salt: Theory and Simulation. The Journal of Physical Chemistry B 105, 10983-10991 (2001).
R. J. Nap et al., in Polymer and Biopolymer Brushes. (John Wiley & Sons, Inc., 2017), pp. 161-221.
D. Wang et al., Why and how nanoparticle's curvature regulates the apparent pKa of the coating ligands. J. Am. Chem. Soc. 133, 2192-2197 (2011).
R. J. Nap, A. Lošdorfer Božič, I. Szleifer, R. Podgornik, The role of solution conditions in the bacteriophage PP7 capsid charge regulation. Biophys. J. 107, 1970-1979 (2014).
D. E. Westmoreland, R. J. Nap, F. Arcudi, I. Szleifer, E. A. Weiss, pH-Dependent structure of water-exposed surfaces of CdSe quantum dots. Chem Commun 55, 5435-5438 (2019).
R. J. Nap et al., Acid-Base Equilibrium and Dielectric Environment Regulate Charge in Supramolecular Nanofibers. Front Chem 10, (2022).
S. Kewalramani et al., Electrolyte-Mediated Assembly of Charged Nanoparticles. Acs Central Sci 2, 219-224 (2016).
K. Krishnamoorthy et al., Defining the Structure of a Protein-Spherical Nucleic Acid Conjugate and Its Counterionic Cloud. Acs Central Sci 4, 378-386 (2018).
M. Gebala et al., Cation-Anion Interactions within the Nucleic Acid Ion Atmosphere Revealed by Ion Counting. J Am Chem Soc 137, 14705-14715 (2015).
S. J. Weigand, D. T. Keane, DND-CAT's new triple area detector system for simultaneous data collection at multiple length scales. Nucl Instrum Meth A 649, 61-63 (2011).
B. H. Toby, R. B. Von Dreele, GSAS-II: the genesis of a modern open-source all purpose crystallography software package. J Appl Crystallogr 46, 544-549 (2013).
M. D. Hanwell et al., Avogadro: an advanced semantic chemical editor, visualization, and analysis platform. J Cheminform 4, 17 (2012).
S. J. Marrink, A. H. de Vries, A. E. Mark, Coarse Grained Model for Semiquantitative Lipid Simulations. The Journal of Physical Chemistry B 108, 750-760 (2004).
D. H. de Jong et al., Improved Parameters for the Martini Coarse-Grained Protein Force Field. Journal of Chemical Theory and Computation 9, 687-697 (2013).
L. Monticelli et al., The MARTINI Coarse-Grained Force Field: Extension to Proteins. Journal of Chemical Theory and Computation 4, 819-834 (2008).
S. J. Marrink, H. J. Risselada, S. Yefimov, D. P. Tieleman, A. H. de Vries, The MARTINI Force Field: Coarse Grained Model for Biomolecular Simulations. The Journal of Physical Chemistry B 111, 7812-7824 (2007).
O.-S. Lee, V. Cho, G. C. Schatz, Modeling the Self-Assembly of Peptide Amphiphiles into Fibers Using Coarse-Grained Molecular Dynamics. Nano Letters 12, 4907-4913 (2012).
C. Tang, A. M. Smith, R. F. Collins, R. V. Ulijn, A. Saiani, Fmoc-Diphenylalanine Self-Assembly Mechanism Induces Apparent pKa Shifts. Langmuir 25, 9447-9453 (2009).
P. W. Frederix et al., Exploring the sequence space for (tri-)peptide self-assembly to design and discover new hydrogels. Nat Chem 7, 30-37 (2015).
C. Guo, Y. Luo, R. Zhou, G. Wei, Probing the self-assembly mechanism of diphenylalanine-based peptide nanovesicles and nanotubes. ACS Nano 6, 3907-3918 (2012).
E. Mayans et al., Self-Assembly of Tetraphenylalanine Peptides. Chemistry 21, 16895-16905 (2015).
W. Humphrey, A. Dalke, K. Schulten, VMD: visual molecular dynamics. J Mol Graph 14, 33-38, 27-38 (1996).
M. J. Abraham et al., GROMACS: High performance molecular simulations through multi-level parallelism from laptops to supercomputers. SoftwareX 1, 19-25 (2015).
D. H. De Jong, S. Baoukina, H. I. Ingólfsson, S. J. Marrink, Martini straight: boosting performance using a shorter cutoff and GPUs. Comput. Phys. Commun. 199, 1-7 (2016).
S. W. I. Siu, K. Pluhackova, R. A. Bockmann, Optimization of the OPLS-AA Force Field for Long Hydrocarbons. J. Chem. Theory Comput. 8, 1459-1470 (2012).
W. L. Jorgensen, D. S. Maxwell, J. Tirado-Rives, Development and Testing of the OPLS All-Atom Force Field on Conformational Energetics and Properties of Organic Liquids. J. Am. Chem. Soc. 118, 11225-11236 (1996).
Y. Sun, P. A. Kollman, Hydrophobic solvation of methane and nonbond parameters of the TIP3P water model. J. Comput. Chem. 16, 1164-1169 (1995).
B. Qiao, K. C. Littrell, R. J. Ellis, Liquid Worm-like and Proto-Micelles: Water Solubilization in Amphiphile-Oil Solutions. Phys. Chem. Chem. Phys. 20, 12908-12915 (2018).
B. Qiao, G. Ferru, M. Olvera de la Cruz, R. J. Ellis, Molecular Origins of Mesoscale Ordering in a Metalloamphiphile Phase. ACS Cent. Sci. 1, 493-503 (2015).
B. Qiao, T. Demars, M. Olvera de la Cruz, R. J. Ellis, How Hydrogen Bonds Affect the Growth of Reverse Micelles around Coordinating Metal Ions. J. Phys. Chem. Lett. 5, 1440-1444 (2014).
B. Doherty, X. Zhong, S. Gathiaka, B. Li, O. Acevedo, Revisiting OPLS Force Field Parameters for Ionic Liquid Simulations. J. Chem. Theory Comput. 13, 6131-6145 (2017).
J. Mähler, I. Persson, A Study of the Hydration of the Alkali Metal Ions in Aqueous Solution. Inorganic Chemistry 51, 425-438 (2012).

1. A composition comprising peptide amphiphiles (PAs) and organic cations (OCs).

2. (canceled)

3. The composition of claim 1, wherein the PAs and OCs co-assemble into nanofibers.

4. The composition of claim 3, wherein at least 70% of the PAs in the nanofibers are fully ionized.

5. The composition of claim 4, wherein the co-assembly does not result in gelation or aggregation of the nanofibers.

6. The composition of claim 1, wherein the PAs comprise:
(a) a hydrophobic non-peptide segment;
(b) a structural peptide segment; and
(c) a charged peptide segment.

7. The composition of claim 6, wherein the hydrophobic non-peptidic segment comprises an acyl chain, the structural peptide segment is a beta-sheet forming peptide segment, the structural peptide segment is an alanine-, glycine-, and/or valine-rich peptide segment, and/or the charged peptide segment is a glutamate- and/or aspartate-rich segment.

8. The composition of claim 7, wherein the acyl chain comprises C₆-C₂₀, the alanine-, glycine-, and/or valine-rich peptide segment comprises AAVV (SEQ ID NO: 1), AAAVVV (SEQ ID NO: 2) VVAA (SEQ ID NO: 3), VVVAAA (SEQ ID NO: 4), AAGG (SEQ ID NO: 5), and/or GGAA (SEQ ID NO: 6), and/or the glutamate- and/or aspartate-rich segment comprises 2-7 amino acids in length with 50% or more amino acids selected from Glu (E) and/or Asp (D) residues.

9. (canceled)

10. (canceled)

11. (canceled)

12. (canceled)

13. (canceled)

14. The composition of claim 8, wherein the glutamate- and/or aspartate-rich segment comprises EE, EEE, or EEEE.

15. The composition of claim 1, wherein the PAs comprise the sequence VVAAEE (SEQ ID NO: 7) or AAGGEEEE (SEQ ID NO: 8).

16. The composition of claim 6, wherein all or a portion of the PAs further comprise (d) a bioactive peptide.

17. The composition of claim 16, wherein the bioactive peptide is selected from a VEGF mimetic peptide (e.g., IKVAV (SEQ ID NO: 9)), a FGF2 mimetic peptide, a TGF-β1 mimetic peptide, BDNF mimetic peptide, Netrin-1 peptide, and BMP-2 binding peptide (e.g., TSPHVPYGGGS (SEQ ID NO: 10)).

18. The composition of claim 16, wherein PAs with a bioactive peptide and PAs without a bioactive peptide are present at a ratio between about 1:10 and about 10:1.

19. The composition of claim 18, wherein PAs with a bioactive peptide and PAs without a bioactive peptide are present at a ratio between about 1:2 and about 2:1.

20. The composition of claim 1, wherein the OCs are selected from 1-ethyl-3-methylimidazolium, ethyl pyridinium, N-ethyl-N-methyl pyrrolidinium, trimethylsulfonium, tetramethylammonium, methylammonium, formamidinium, aziridiium, cyclopropenium, ethylenediamine, allyl methyl imidazolium, butyl methyl imidazolium, and ethyl methyl imidazolium, or wherein the OCs are small molecules having molecular weights of 500 g/mol or less, or wherein the OCs are monocationic.

21. (canceled)

22. (canceled)

23. (canceled)

24. The composition of claim 1, wherein the composition comprises about 1 to about 50 molar equivalents of OC per PA.

25. The composition of claim 1, further comprising one or more types of inorganic cations ions, wherein the inorganic cations form an ion cloud around the nanofibers.

26. The composition of claim 25, wherein the inorganic cations monoanionic, or wherein the inorganic cations are selected from K⁺, Na⁺, Rb⁺, Cs⁺, Ca²⁺, Mg²⁺, Zn²⁺, Sc³⁺, and Mn²⁺.

27. (canceled)

28. (canceled)

29. The composition of claim 6, further comprising an organic cation.

30. The composition of claim 29, wherein the peptide amphiphile further comprises a bioactive peptide segment, or wherein the composition further comprises a second peptide amphiphile comprising (i) a hydrophobic non-peptide segment, (ii) a structural peptide segment, (iii) a charged peptide segment, and (iv) a bioactive peptide segment.

31. (canceled)

32. A method of enhancing the development and/or membrane excitability of neurons in vitro comprising contacting the neurons with the composition of claim 1.

33. (canceled)

34. (canceled)

35. (canceled)
